(19) United States
(12) Patent Application Publication
(10) Pub. No.: US 2008/0249414 A1
(43) Pub. Date: Oct. 9, 2008

(54) SYSTEM AND METHOD TO MEASURE CARDIAC EJECTION FRACTION

(76) Inventors: Fuxing Yang, Woodinville, WA (US); Jongtae Yuk, Redmond, WA (US); Vikram Chalana, Mill Creek, WA (US); Steven J. Shankle, Kirkland, WA (US); Stephen Dudycha, Bothell, WA (US); Gerald J. McMorrow, Kirkland, WA (US)

Correspondence Address: BLACK LOWE & GRAHAM, PLLC, 701 FIFTH AVENUE, SUITE 4800, SEATTLE, WA, 98104, US

(21) Appl. No.: 11/925,896

(22) Filed: Oct. 27, 2007

Related U.S. Application Data
(63) Continuation of application No. 11/132,076, filed on May 17, 2005.
Application No. 11/132,076 is a continuation-in-part of application No. 11/119,355, filed on Apr. 29, 2005.
Application No. 11/119,355 is a continuation-in-part of application No. 10/701,955, filed on Nov. 5, 2003, now Pat. No. 7,087,022.
Application No. 11/119,355 is a continuation-in-part of application No. 10/443,126, filed on May 20, 2003, now Pat. No. 7,041,059.
Application No. 11/132,076 is a continuation-in-part of application No. 11/061,867, filed on Feb. 17, 2005.
Application No. 11/061,867 is a continuation-in-part of application No. 10/704,966, filed on Nov. 12, 2003, now Pat. No. 6,803,308.
Application No. 11/061,867 is a continuation-in-part of application No. PCT/US03/24368, filed on Aug. 1, 2003.
Application No. 11/132,076 is a continuation-in-part of application No. PCT/US03/14785, filed on May 9, 2003.
Application No. PCT/US03/14785 is a continuation of application No. 10/165,556, filed on Jun. 7, 2002, now Pat. No. 6,676,605.
Application No. 10/165,556 is a continuation-in-part of application No. 10/888,735, filed on Jul. 9, 2004, now abandoned.
Application No. 10/888,735 is a continuation-in-part of application No. 10/633,186, filed on Jul. 31, 2003, now Pat. No. 7,004,904.
Application No. 11/132,076 is a continuation-in-part of application No. 10/443,126, filed on May 20, 2003, now Pat. No. 7,041,059.
(60) Provisional application No. 60/571,797, filed on May 17, 2004.
(60) Provisional application No. 60/571,799, filed on May 17, 2004.
(60) Provisional application No. 60/545,576, filed on Feb. 17, 2004.
(60) Provisional application No. 60/566,818, filed on Apr. 30, 2004.
(60) Provisional application No. 60/621,349, filed on Oct. 22, 2004.
(60) Provisional application No. 60/423,881, filed on Nov. 5, 2002.
(60) Provisional application No. 60/400,624, filed on Aug. 2, 2002.
(60) Provisional application No. 60/609,184, filed on Sep. 10, 2004.
(60) Provisional application No. 60/605,391, filed on Aug. 27, 2004.
(60) Provisional application No. 60/608,426, filed on Sep. 9, 2004.
(60) Provisional application No. 60/423,881, filed on Nov. 5, 2002.
(60) Provisional application No. 60/423,881, filed on Nov. 5, 2002.
(60) Provisional application No. 60/400,624, filed on Aug. 2, 2002.

(30) Foreign Application Priority Data
Dec. 24, 2002 (KR) 10-2002-0083525

(51) Int. Cl. A61B 8/14

(52) U.S. Cl. 600/445

(57) ABSTRACT

A system and method to acquire 3D ultrasound-based images during the end-systole and end-diastole time points of a cardiac cycle to allow determination of the change and percentage change in left ventricle volume at the time points.

The following applications are incorporated by reference as if fully set forth herein: U.S. application Ser. Nos. 11/132,076 filed May 17, 2005 and 11/460,182 filed Jul. 26, 2006.

### FIELD OF THE INVENTION

The invention pertains to the field of medical-based ultrasound, more particularly using ultrasound to visualize and/or measure internal organs.

### BACKGROUND OF THE INVENTION

Contractility of cardiac muscle fibers can be ascertained by determining the ejection fraction (EF) output from a heart. The ejection fraction is defined as the ratio between the stroke volume (SV) and the end diastolic volume (EDV) of the left ventricle (LV). The SV is defined to be the difference between the end diastolic volume and the end systolic volume of the left ventricle (LV) and corresponds the amount of blood pumped into the aorta during one beat. Determination of the ejection fraction provides a predictive measure of a cardiovascular disease conditions, such as congestive heart failure (CHF) and coronary heart disease (CHD). Left ventricle ejection fraction has proved useful in monitoring progression of congestive heart disease, risk assessment for sudden death, and monitoring of cardiotoxic effects of chemotherapy drugs, among other uses.

Ejection fraction determinations provide medical personnel with a tool to manage CHF. EF serves as an indicator used by physicians for prescribing heart drugs such as ACE inhibitors or beta-blockers. The measurement of ejection fraction has increased to approximately 81% of patients suffering a myocardial infarction (MI). Ejection fraction also has shown to predict the success of antitachycardia pacing for fast ventricular tachycardia

Currently accepted clinical method for determination of end-diastolic volume (EDV), end-systolic volume (ESV) and ejection fraction (EF) involves use of 2-D echocardiography, specifically the apical biplane disk method. Results of this method are highly dependant on operator skill and the validity of assumptions of ventricle symmetry. Further, existing machines for obtaining echocardiography (ECG)-based data are large, expensive, and inconvenient. Having a less expensive, and optionally portable device that is capable of accurately measuring EF would be more beneficial to a patient and medical staff.

Computer based analysis of medical images pertaining cardiac structures allows diagnosis of cardiovascular diseases. Identifying the heart chambers, the endocardium, epicardium, ventricular volumes, and wall thicknesses during various stages of the cardiac cycle provides the physician to access disease state and prescribe therapeutic regimens. There is a need to non-invasively and accurately derive information of the heart during its beating cycle between systole and diastole.

### SUMMARY OF THE INVENTION

Preferred embodiments use three dimensional (3D) ultrasound to acquire at least one 3D image or data set of a heart in order to measure change in volume, preferably at the end-diastolic and end-systole time points as determined by ECG to calculate the ventricular ejection fraction.

The description of image acquisition and processing systems and methods to automatically detect the boundaries of shapes of structures within a region of interest of an image or series of images. The automatically segmented shapes are further image processed to determine thicknesses, areas, volumes, masses and changes thereof as the structure of interest experiences dynamic change.

### BRIEF DESCRIPTION OF THE DRAWINGS

FIG. 1 is a side view of a microprocessor-controlled, hand-held ultrasound transceiver;

FIG. 2A is a is depiction of a hand-held transceiver in use for scanning a patient;

FIG. 2B is a perspective view of a hand-held transceiver device sitting in a communication cradle;

FIG. 3 is a perspective view of a cardiac ejection fraction measuring system;

FIG. 4 is an alternate embodiment of a cardiac ejection fraction measuring system in schematic view of a plurality of transceivers in connection with a server;

FIG. 5 is another alternate embodiment of a cardiac ejection fraction measuring system in a schematic view of a plurality of transceivers in connection with a server over a network;

FIG. 6A a graphical representation of a plurality of scan lines forming a single scan plane;

FIG. 6B is a graphical representation of a plurality of scanplanes forming a three-dimensional array having a substantially conical shape;

FIG. 6C is a graphical representation of a plurality of 3D distributed scanlines emanating from a transceiver forming a scancone;

FIG. 7 is a cross sectional schematic of a heart;

FIG. 8 is a graph of a heart cycle;

FIG. 9 is a schematic depiction of a scanplane overlaid upon a cross section of a heart;

FIG. 10A is a schematic depiction of an ejection fraction measuring system deployed on a subject;

FIG. 10B is a pair of ECG plots from a system of FIG. 10A;

FIG. 11 is a schematic depiction of expanded details of a particular embodiment of an ejection fraction measuring system of FIG. 10A;

FIG. 12 shows a block diagram overview of a method to visualize and determine the volume or area of the cardiac ejection fraction; and

FIG. 13 is a block diagram algorithm overview of registration and correcting algorithms for multiple image cones for determining cardiac ejection fraction.

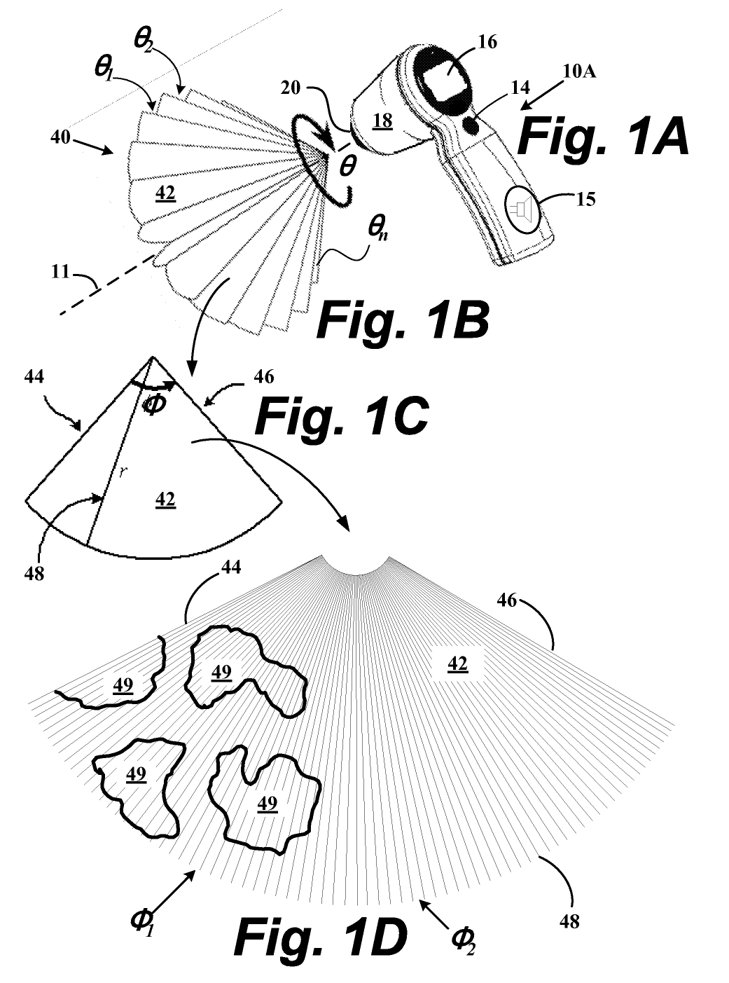

FIGS. 1A-D depicts a partial schematic and a partial isometric view of a transceiver, a scan cone comprising a rotational array of scan planes, and a scan plane of the array;

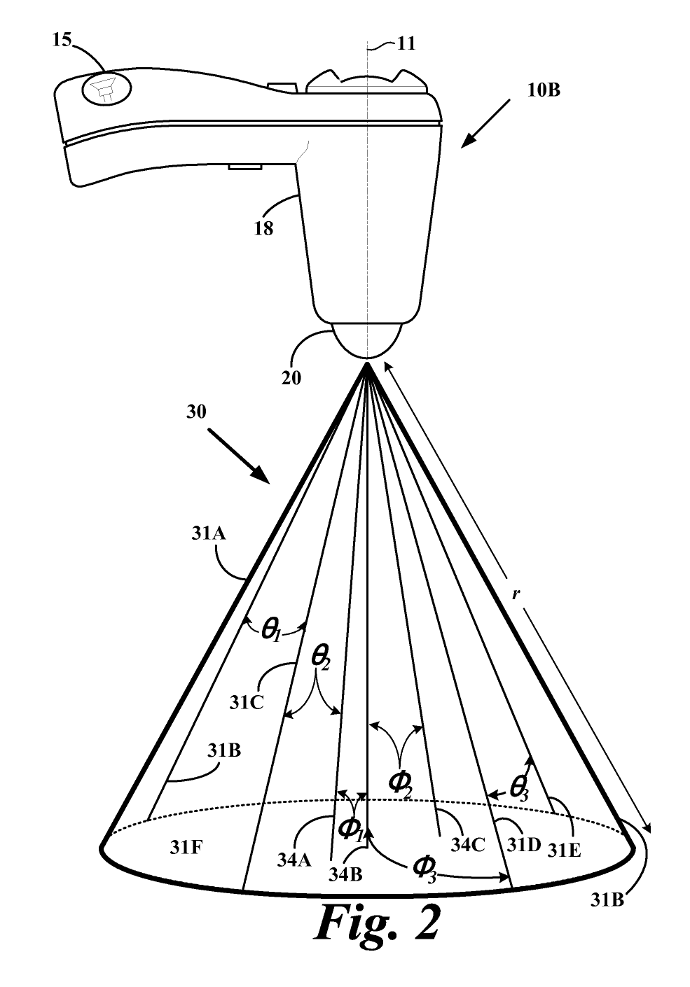

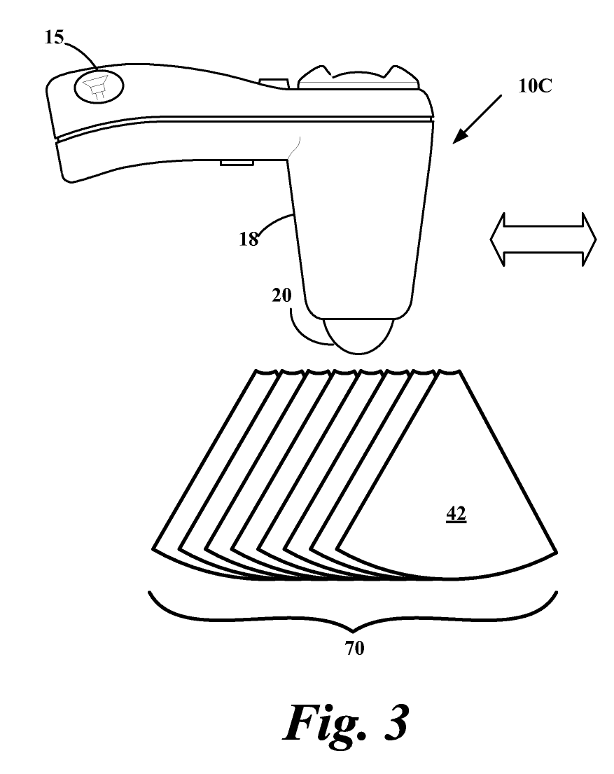

FIG. 2 depicts a partial schematic and partial isometric and side view of a transceiver, and a scan cone array comprised of 3D-distributed scan lines;

FIG. 3 depicts a transceiver 10C acquiring a translation array 70 of scanplanes 42;

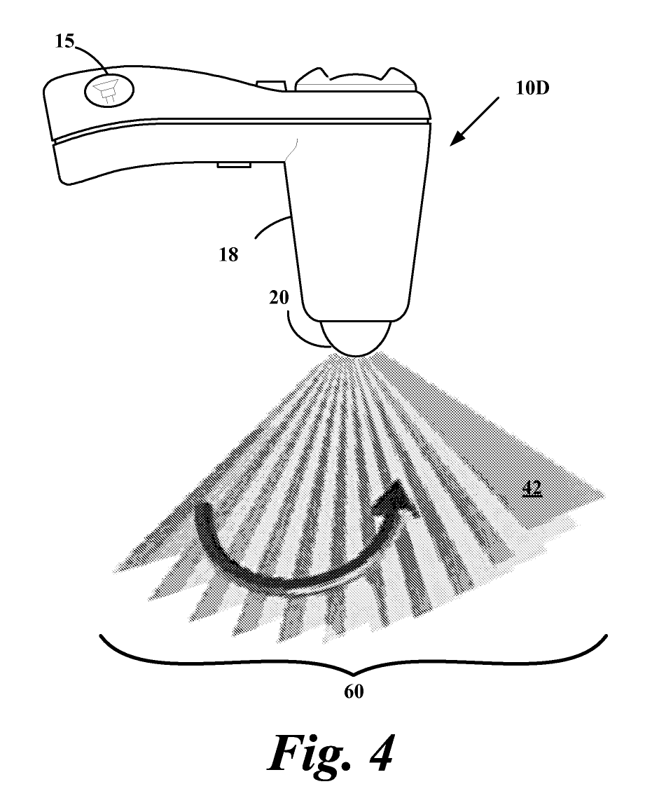

FIG. 4 depicts a transceiver 10D acquiring a fan array 60 of scanplanes 42;

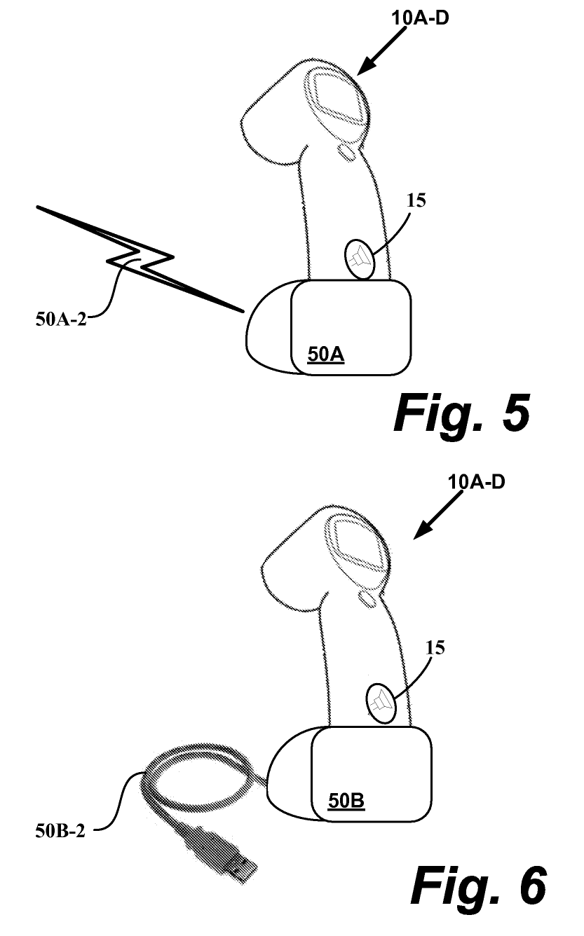

FIG. 5 depicts the transceivers 10A-D (FIG. 1) removably positioned in a communications cradle 50A that is operable to communicate the data wirelessly uploaded to the computer or other microprocessor device (not shown);

FIG. 6 depicts the transceivers 10A-D removably positioned in a communications cradle to communicate imaging data by wire connections uploaded to the computer or other microprocessor device (not shown);

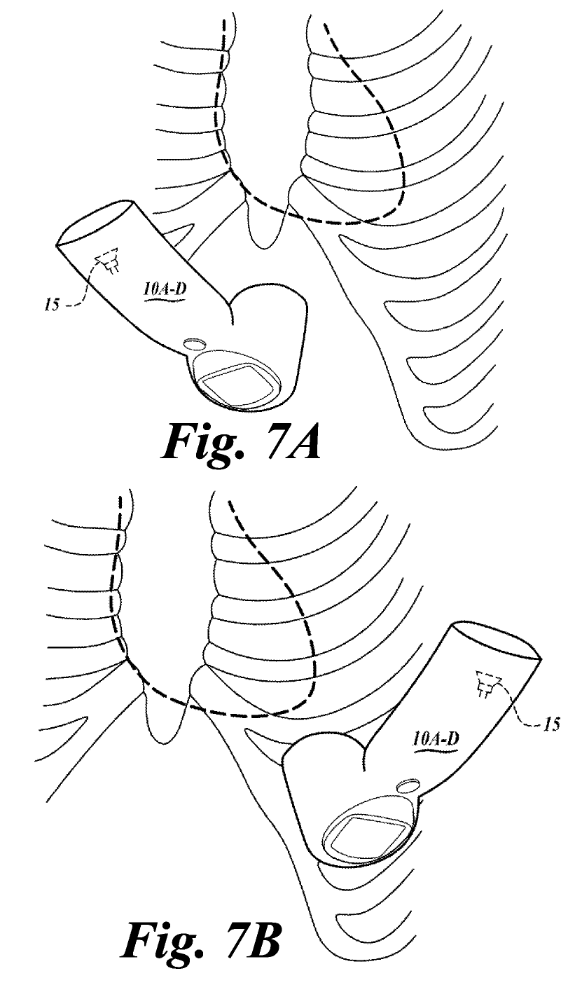

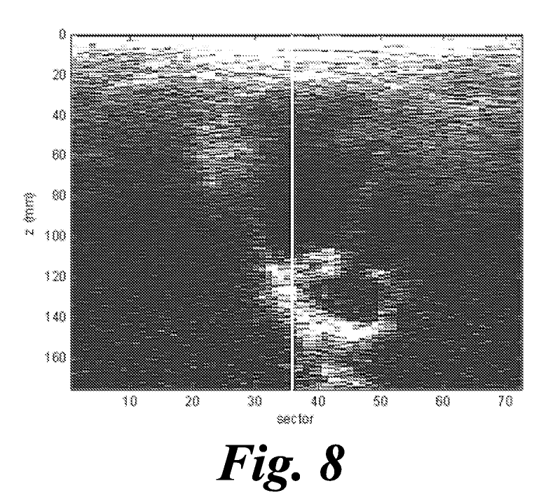

FIG. 7A depicts an image showing the chest area of a patient 68 being scanned by a transceivers 10A-D at a first freehand position and the data being wirelessly uploaded to a personal computer during initial targeting of a cardiac region of interest (ROI);

FIG. 7B depicts an image showing the chest area of the patient 68 being scanned by a transceiver 10A-D at a second freehand position where the transceiver 10A-D is aimed toward the cardiac ROI between ribs of the left side of the thoracic cavity;

FIG. 8 depicts the centering of the heart for later acquisition of 3D image sets based upon the placement of the mitral valve near the image center as determined by the characteristic Doppler sounds from the speaker 15 of transceivers 10A-D.

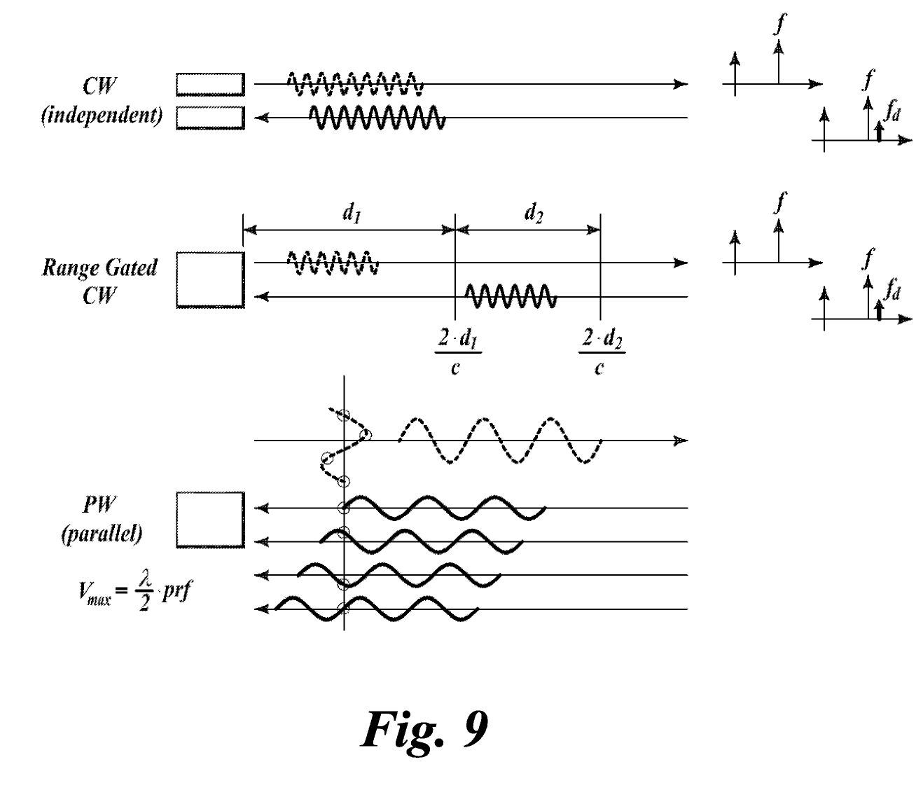

FIG. 9 is a schematic depiction of the Doppler operation of the transceivers 10A-D;

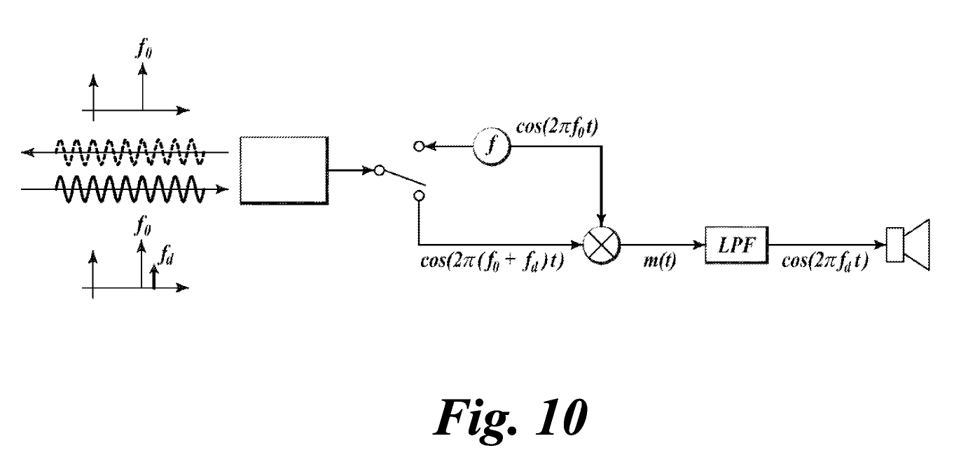

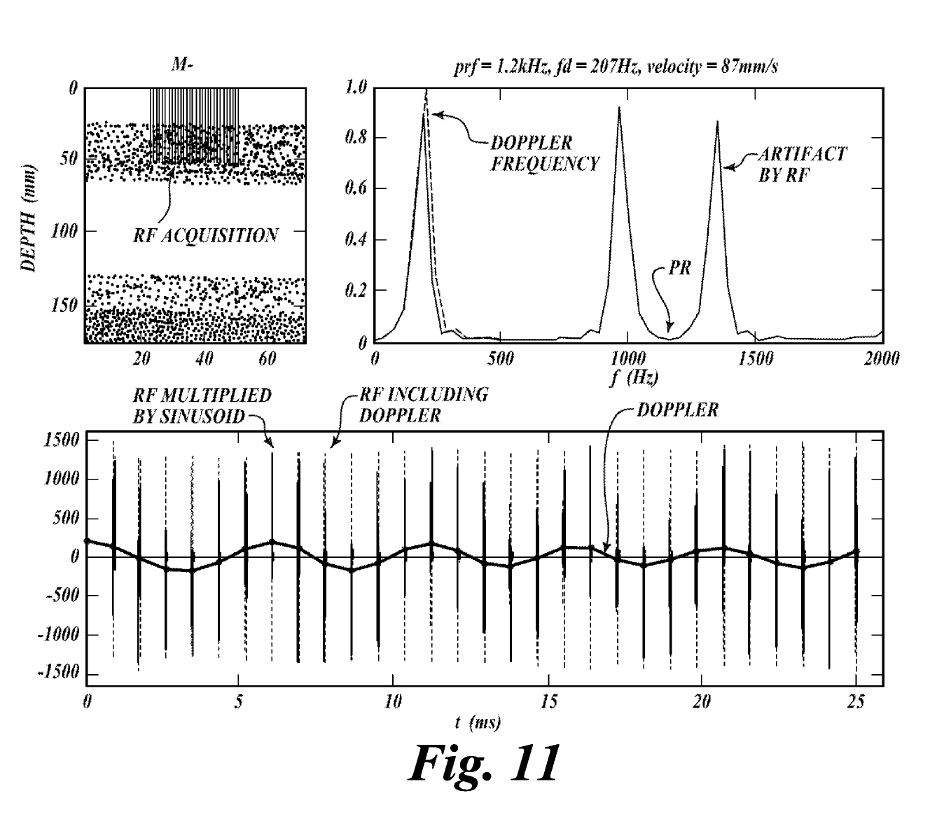

FIG. 10 is a system schematic of the Doppler-speaker circuit of the transceivers 10A-D;

FIG. 11 presents three graphs describing the operation of image acquisition using radio frequency ultrasound (RFUS) and timing to acquire RFUS images at cardiac systole and diastole to help determine the cardiac ejection fractions of the left and/or right ventricles;

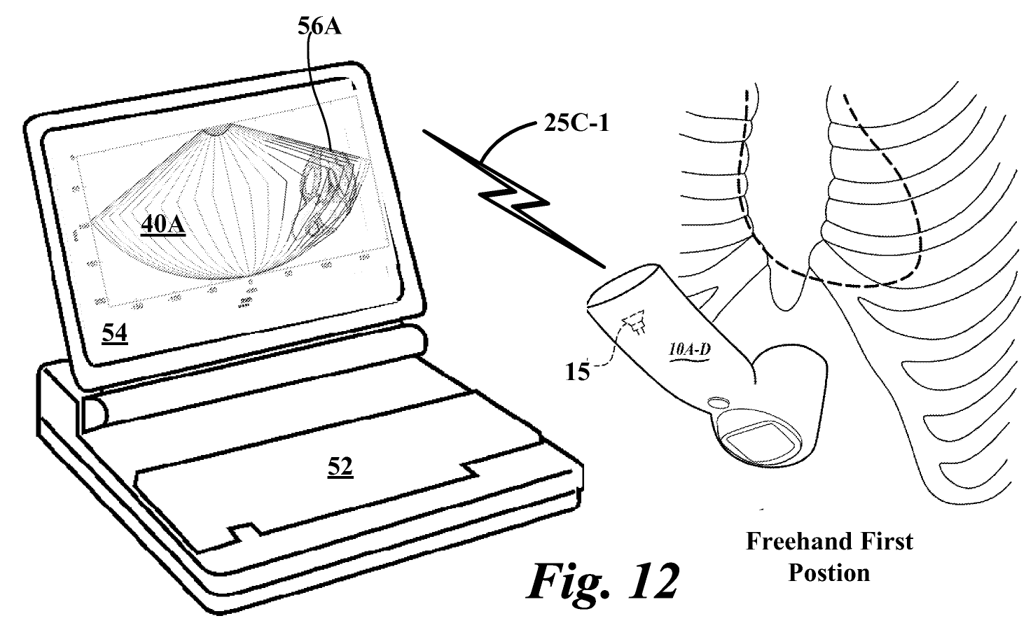

FIG. 12 depicts an alternate embodiment of the cardiac imaging system using an electrocardiograph in communication with a wireless ultrasound transceiver displaying an off-centered cardiac region of interest (ROI);

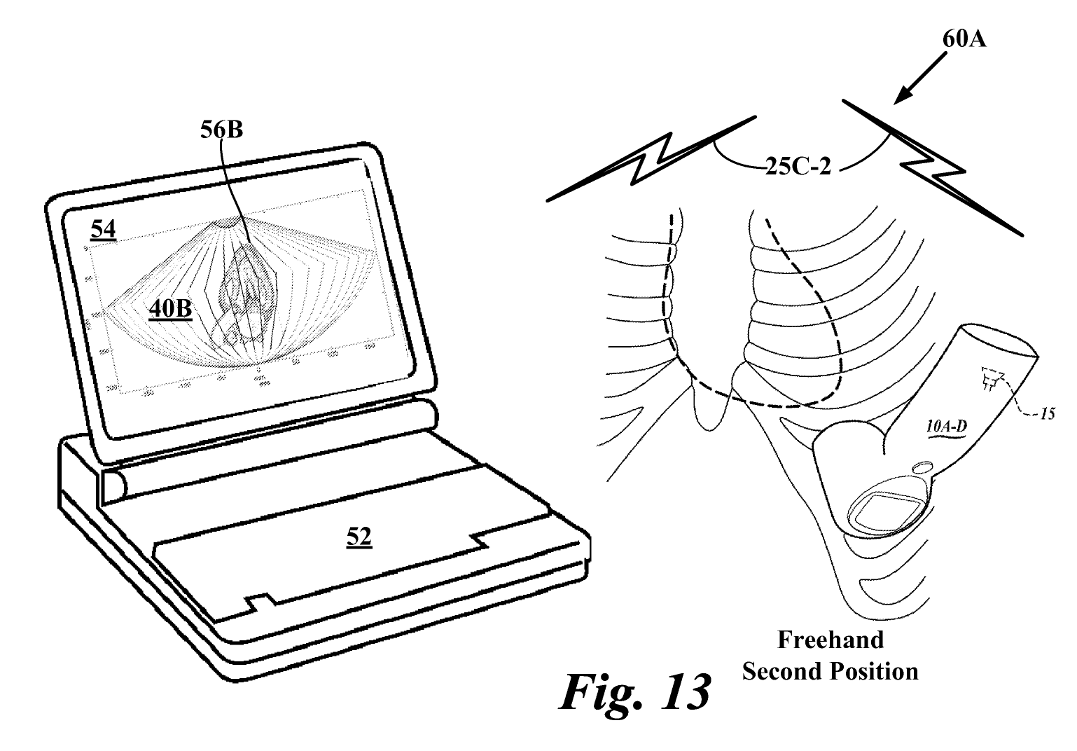

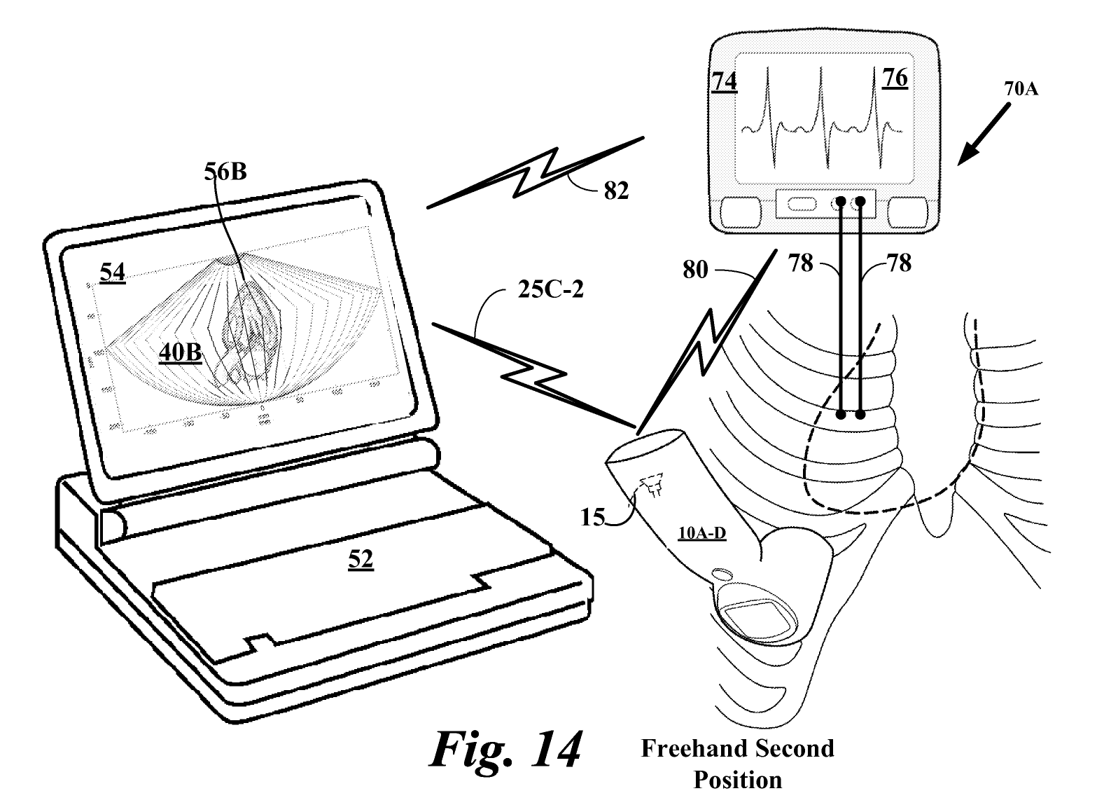

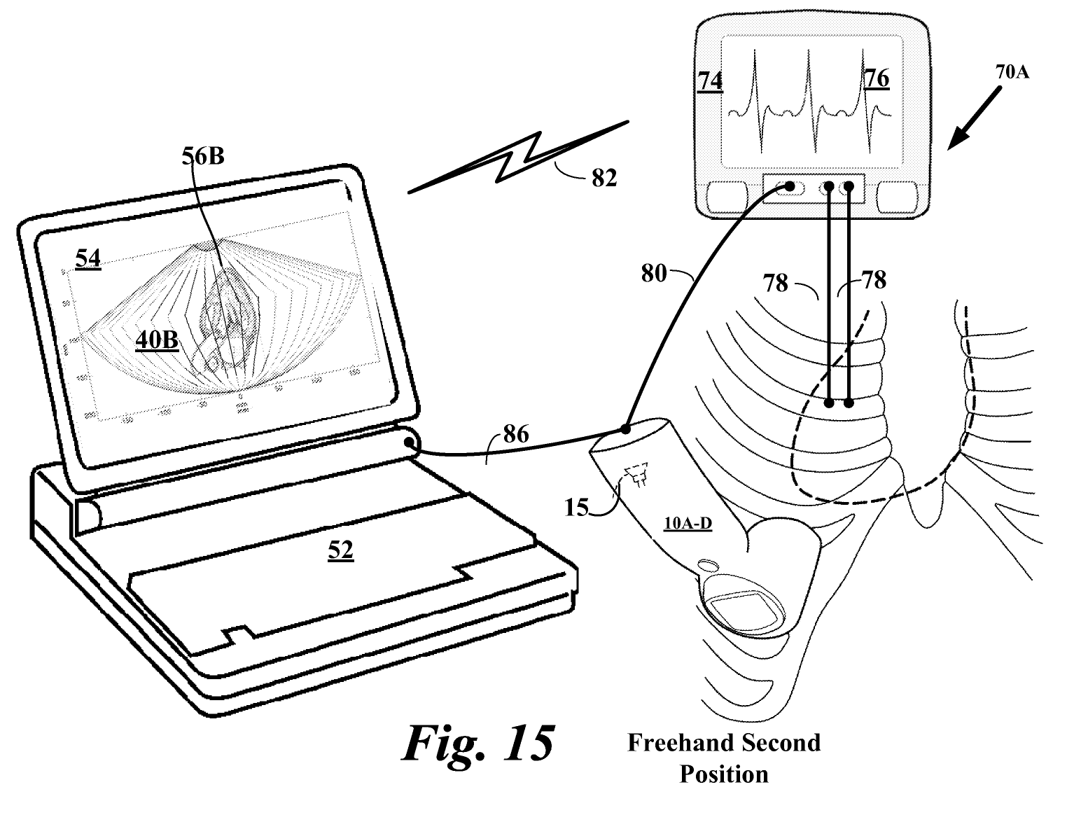

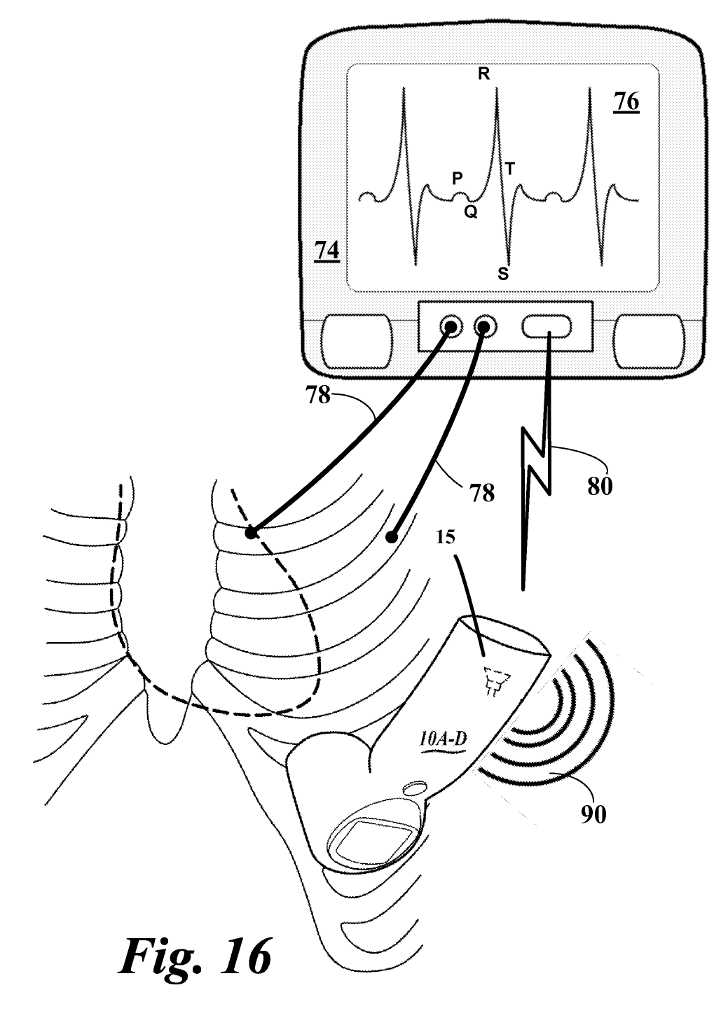

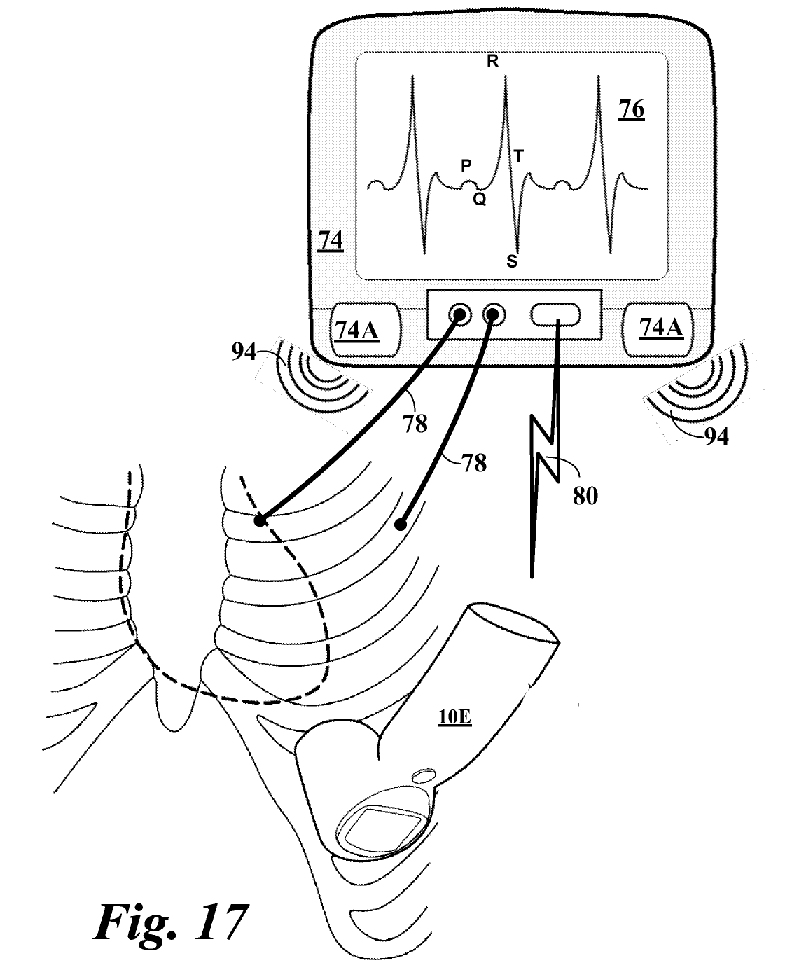

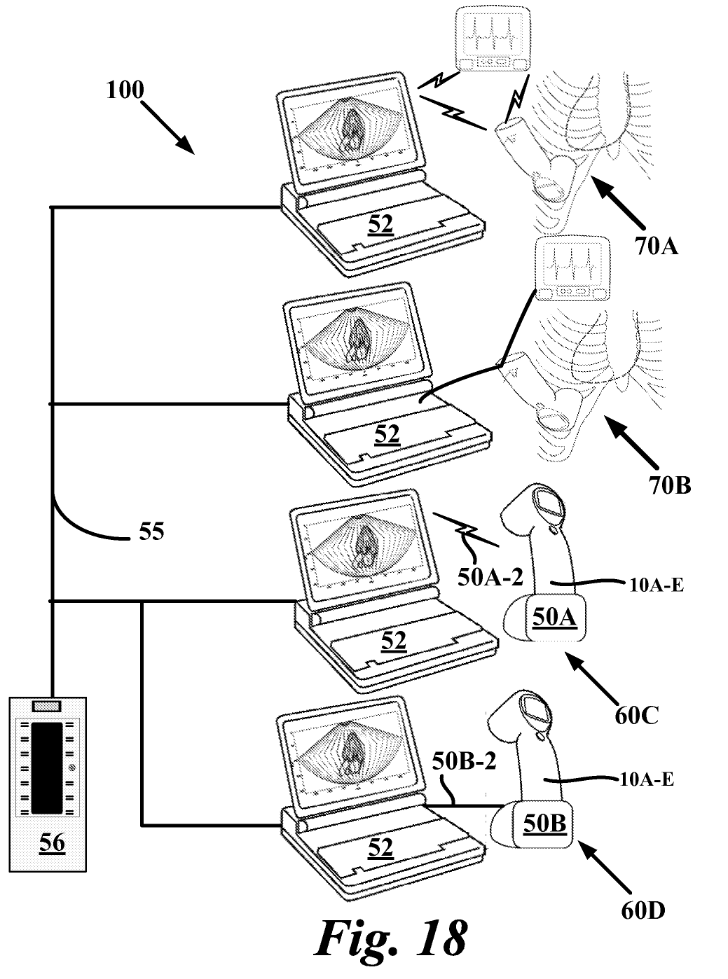

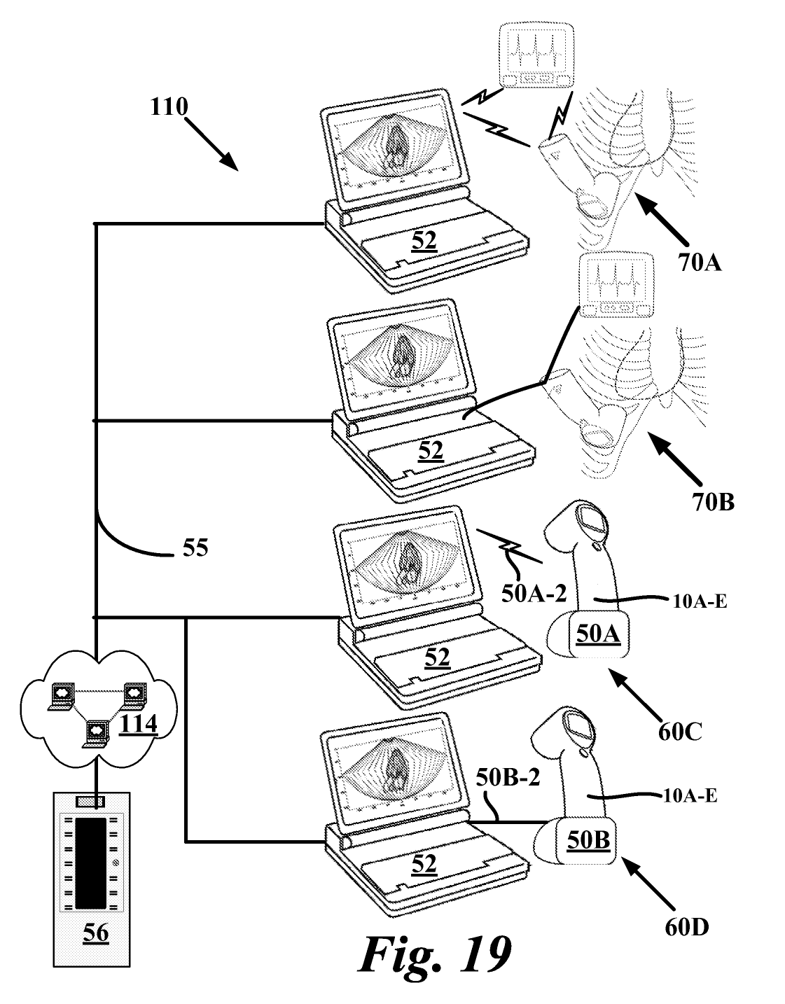

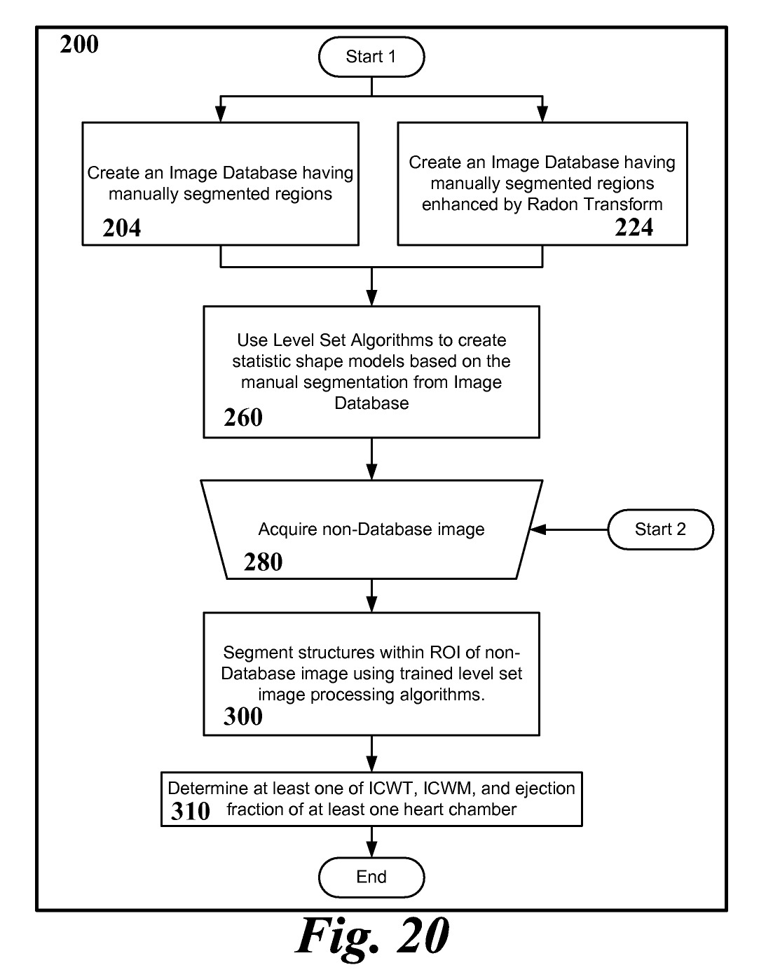

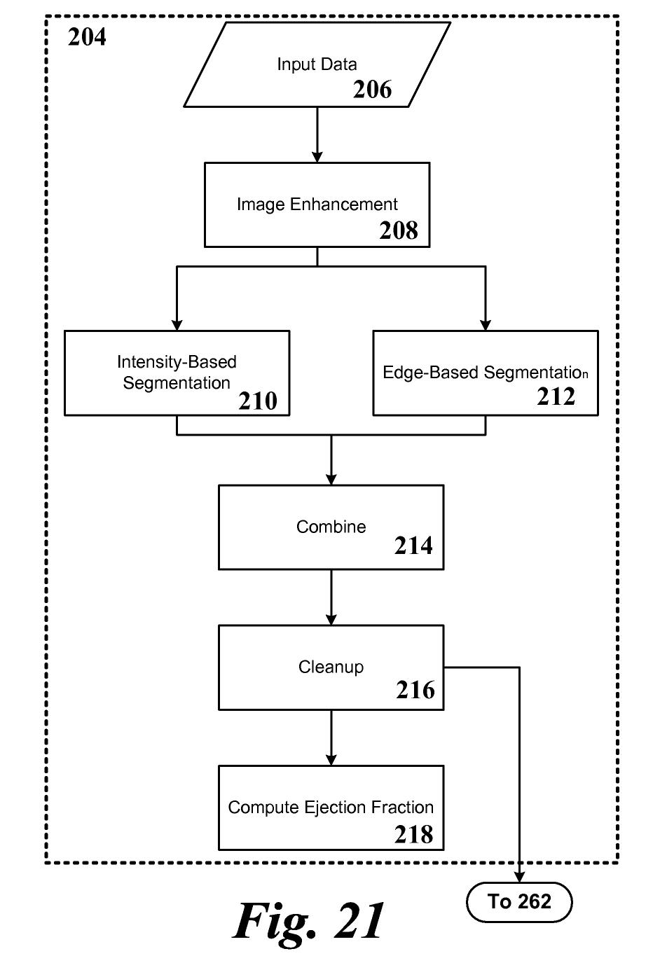

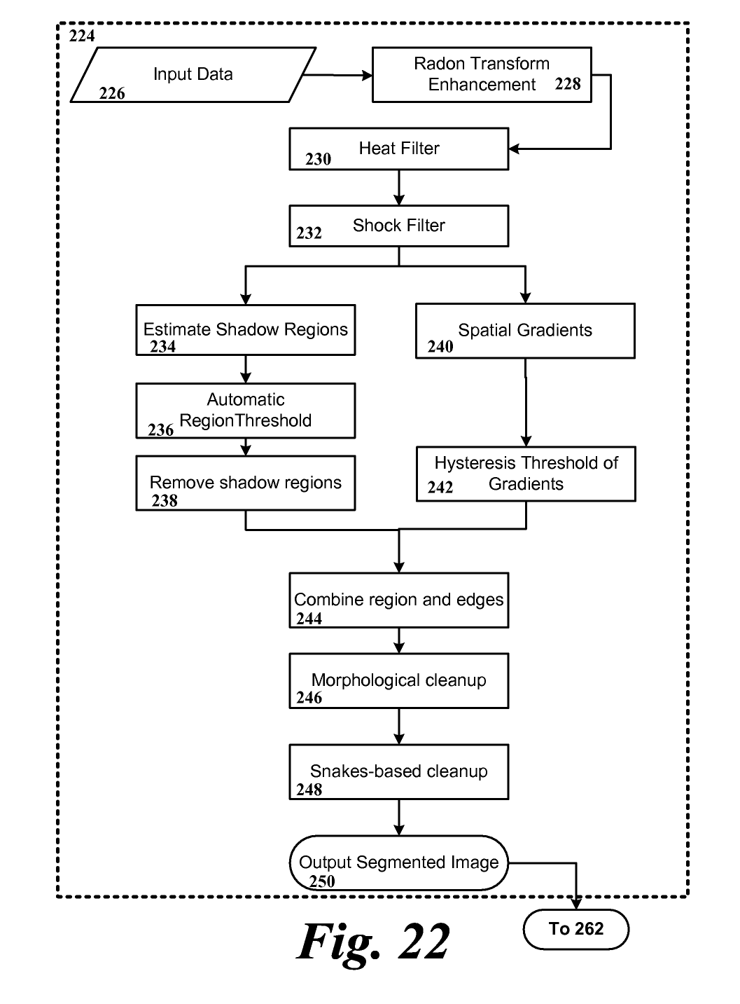

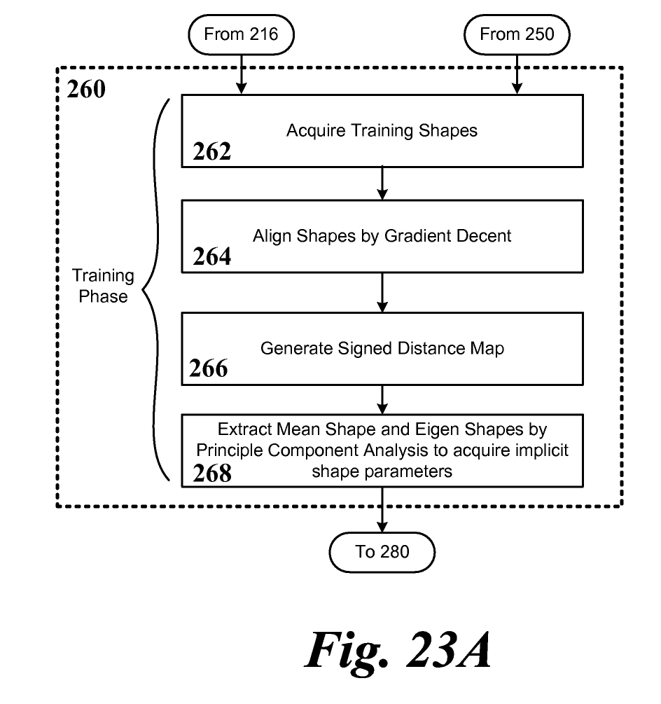

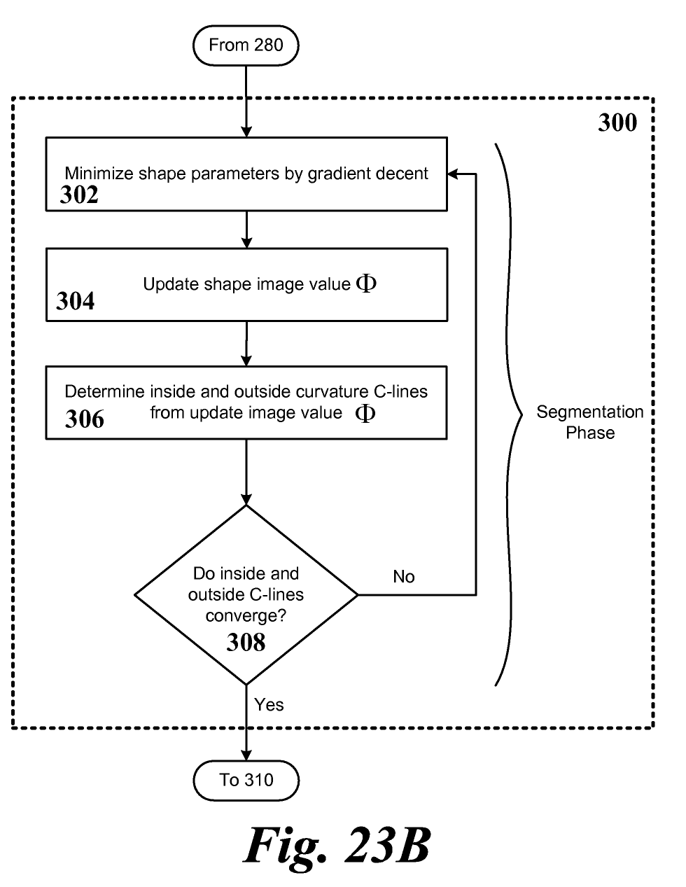

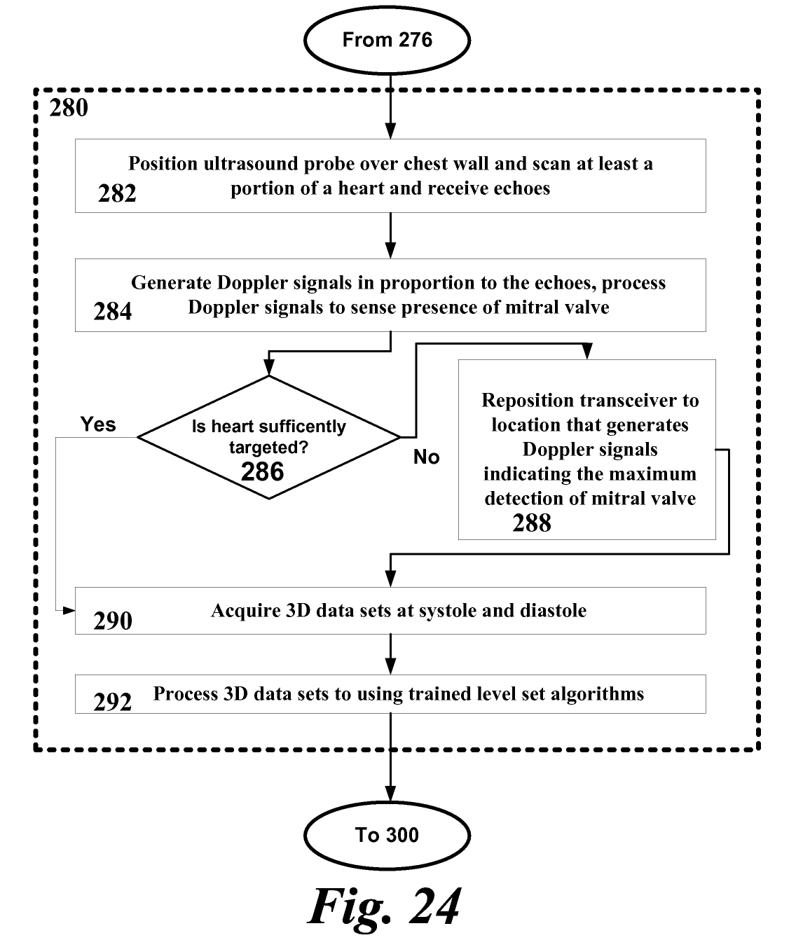

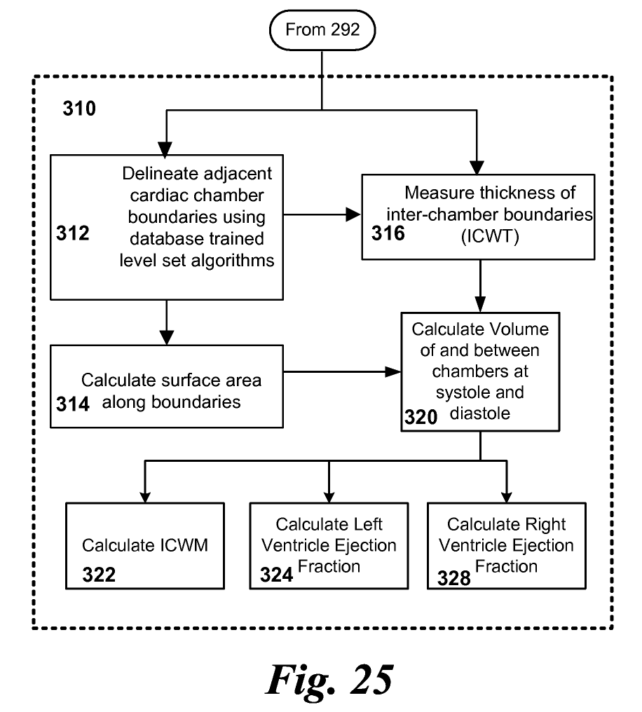

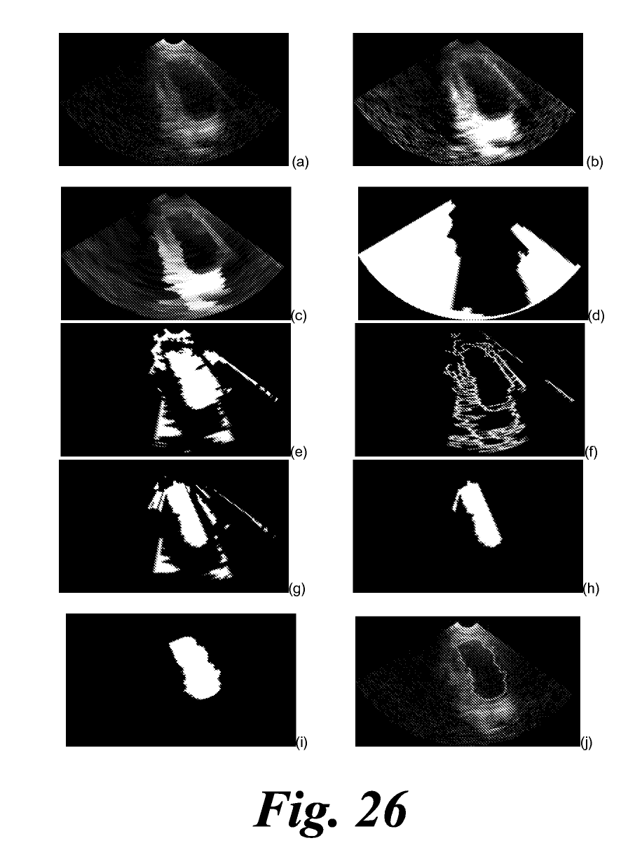

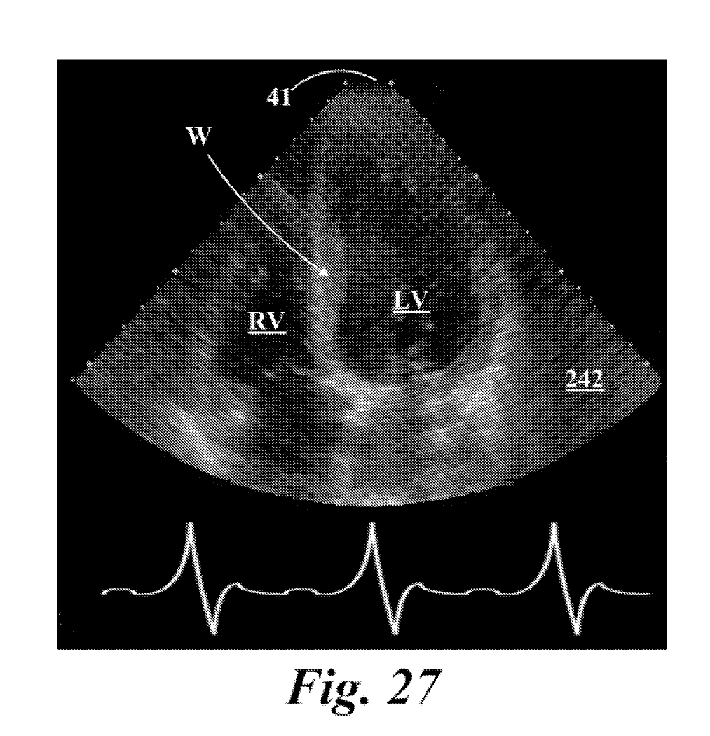

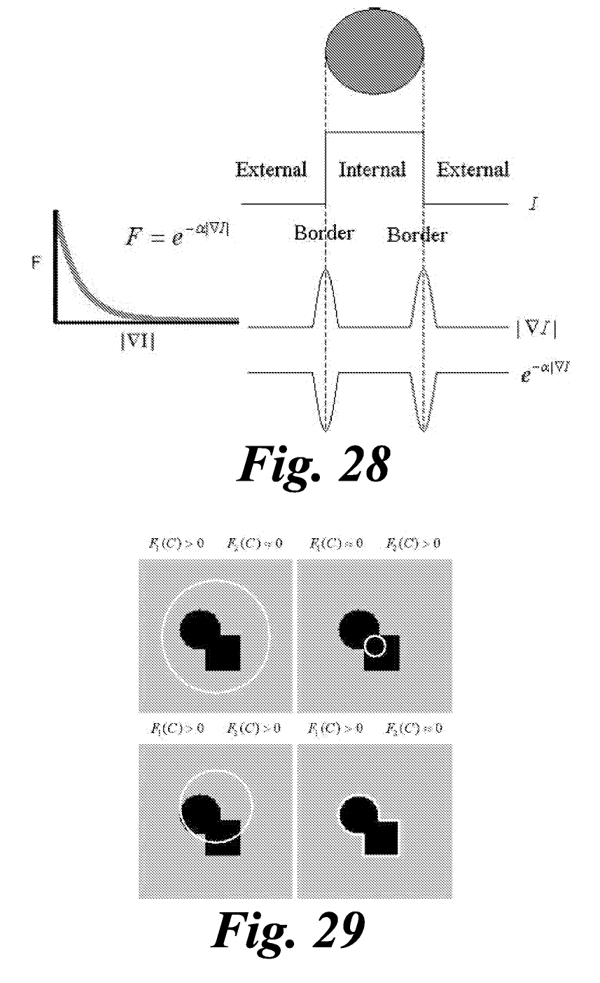

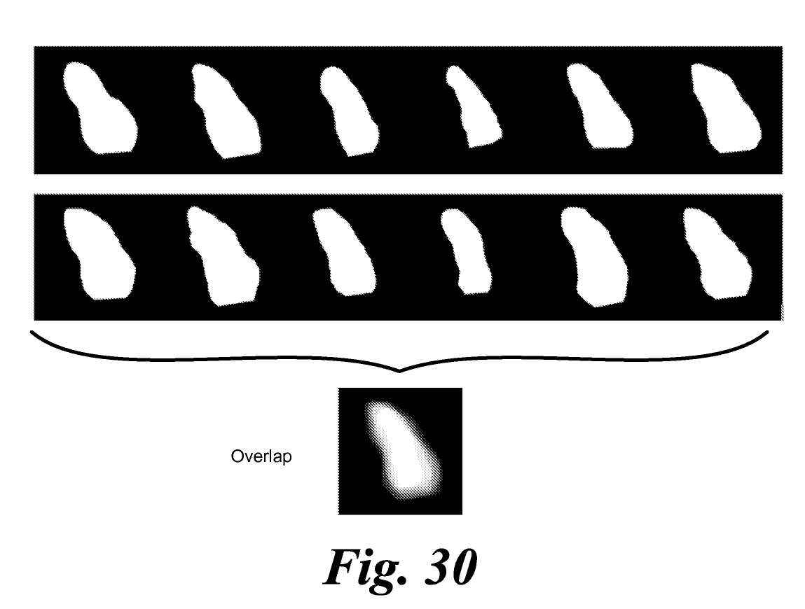

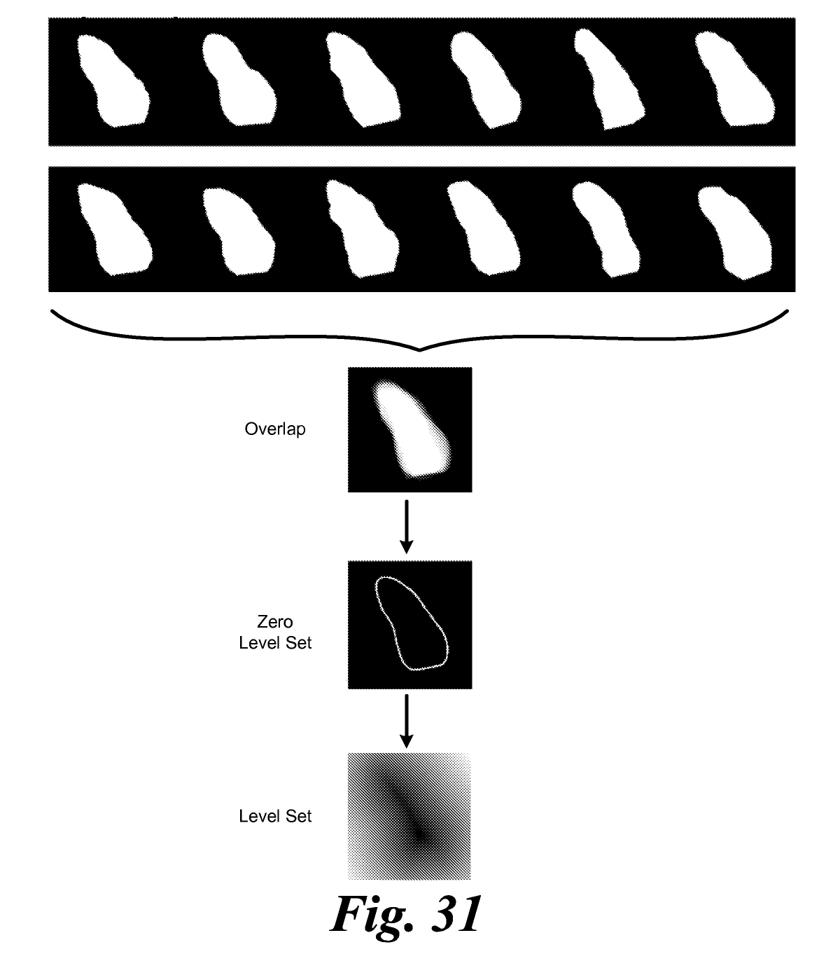

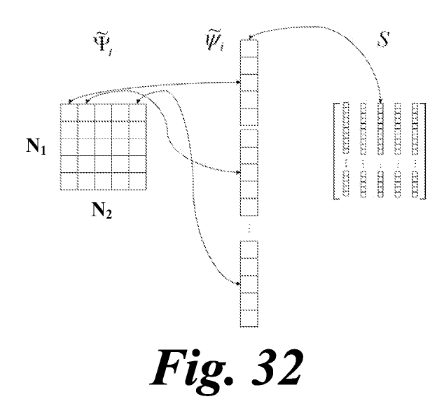

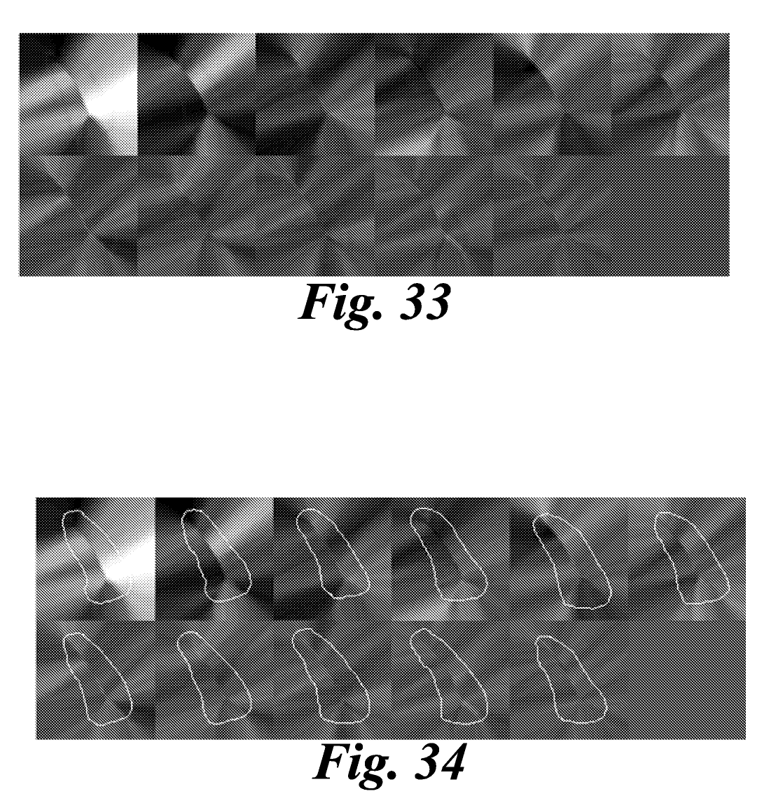

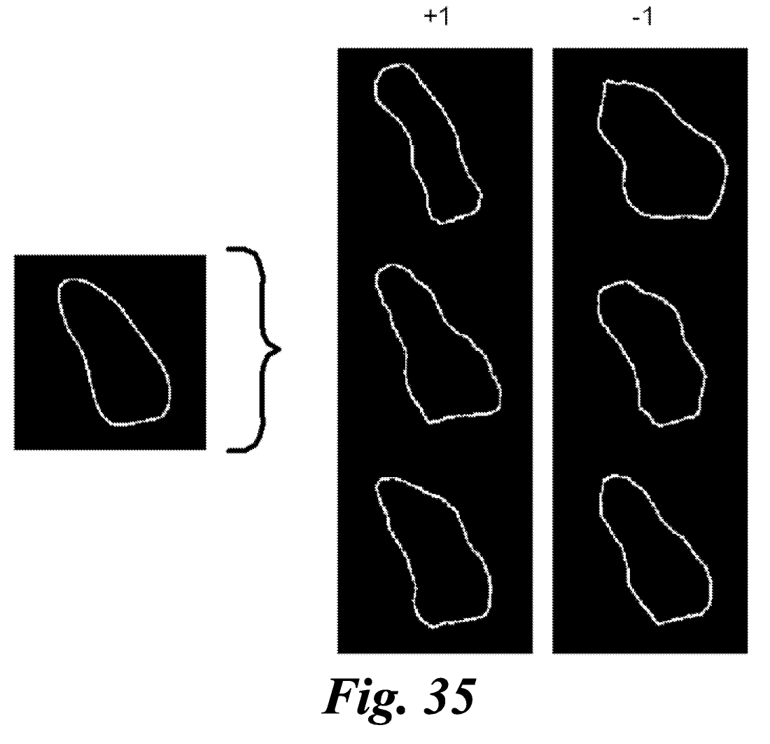

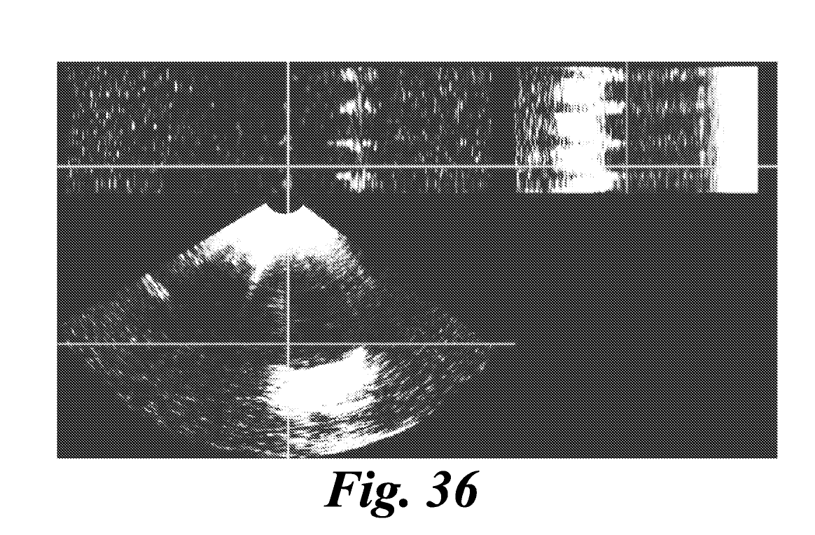

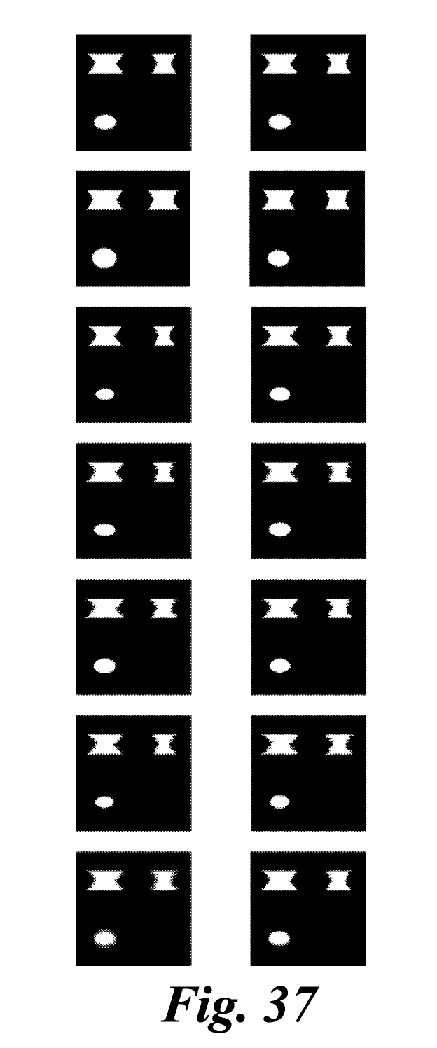

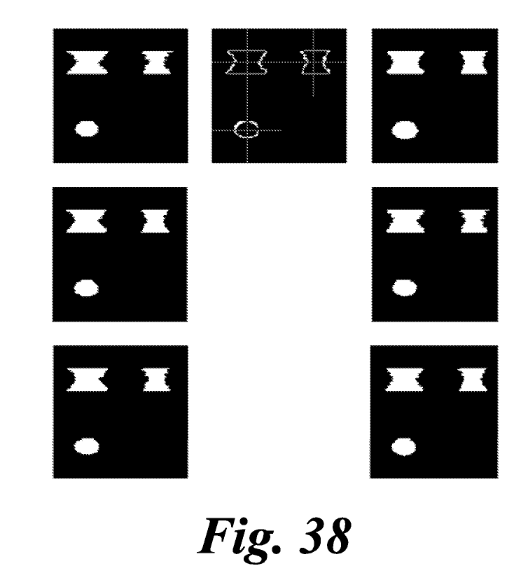

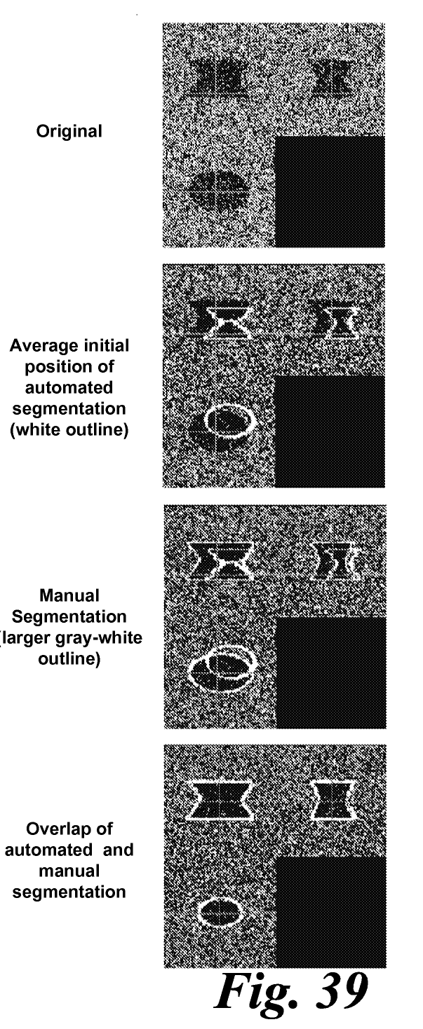

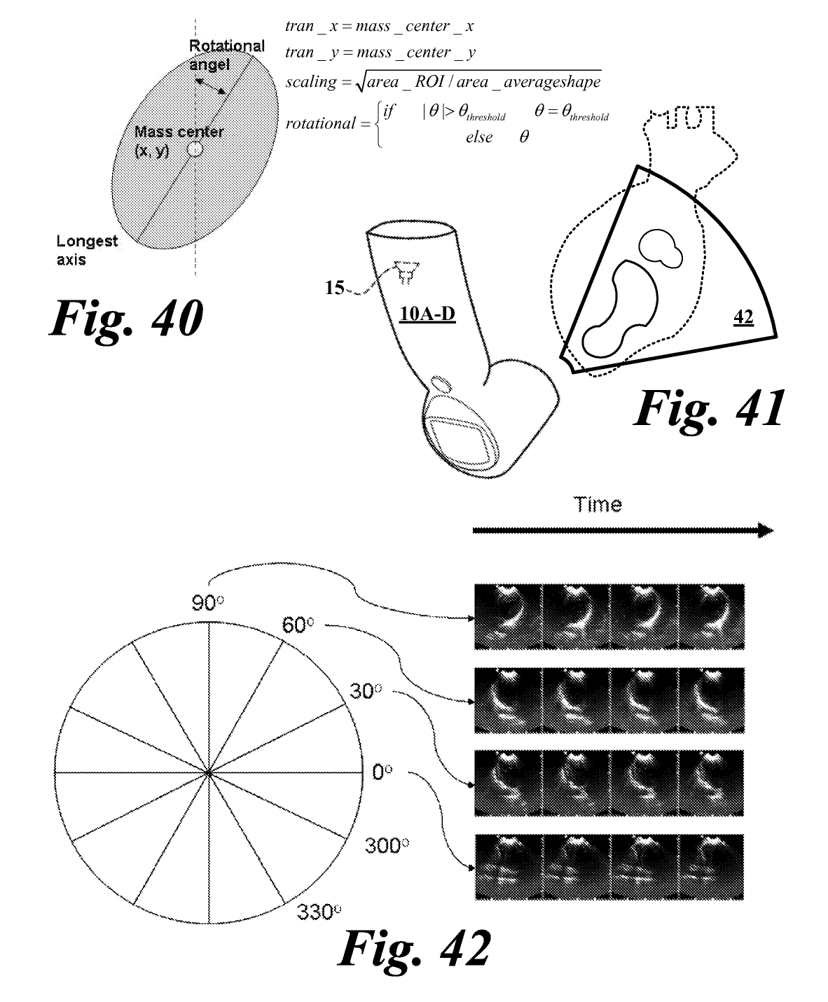

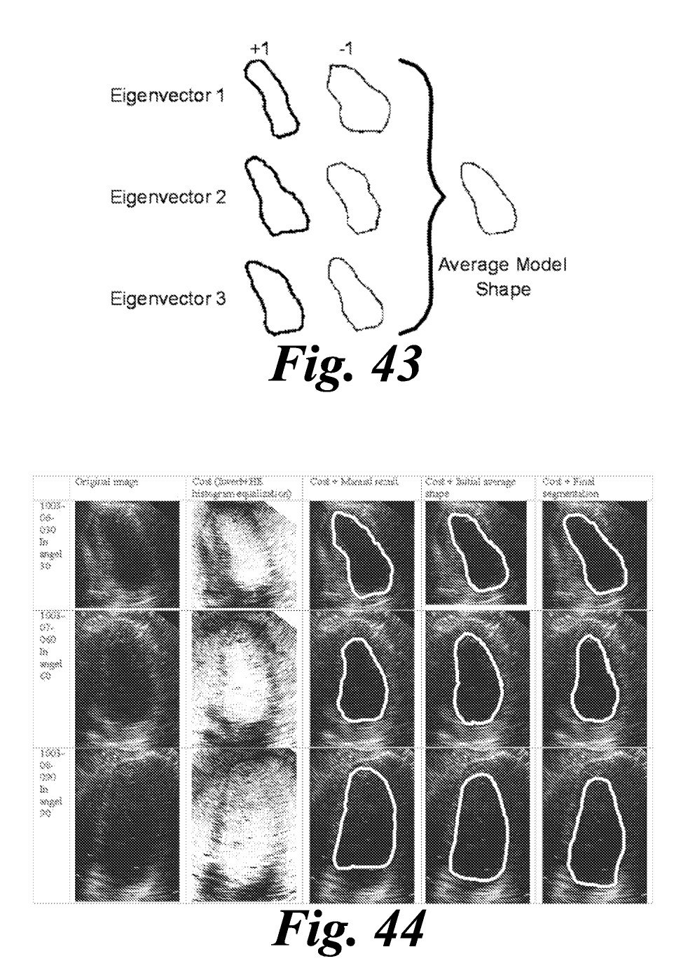

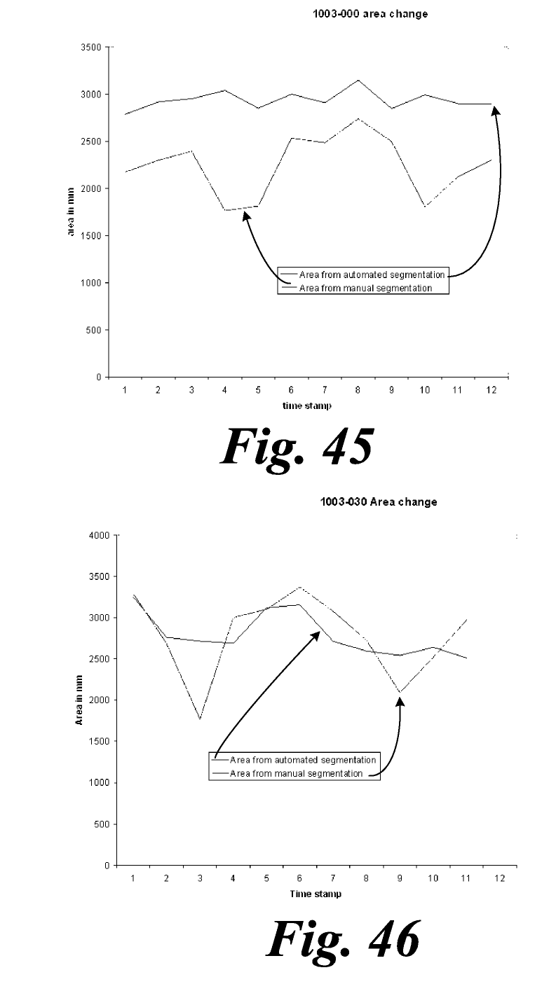

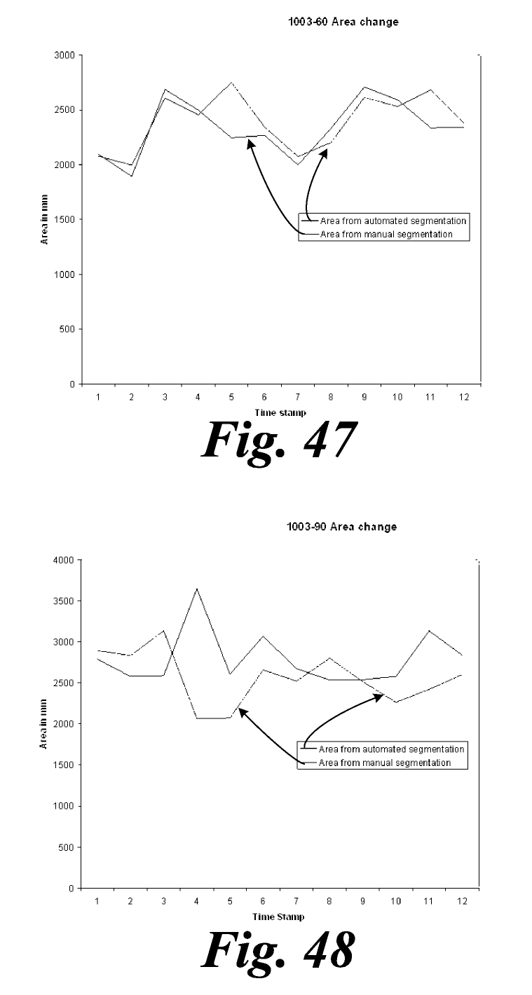

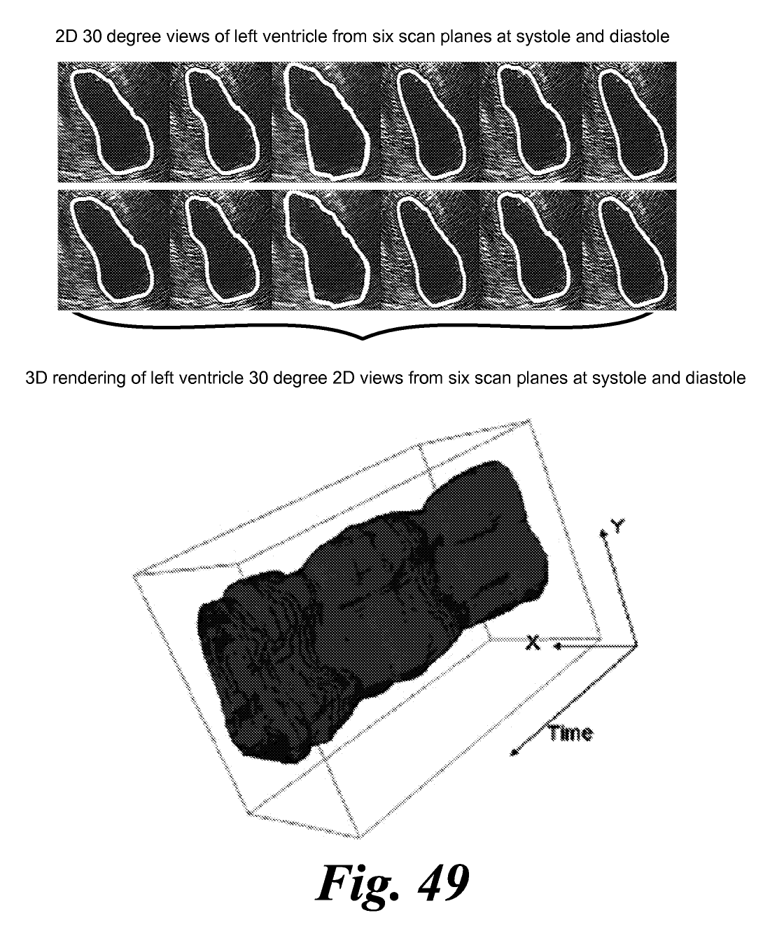

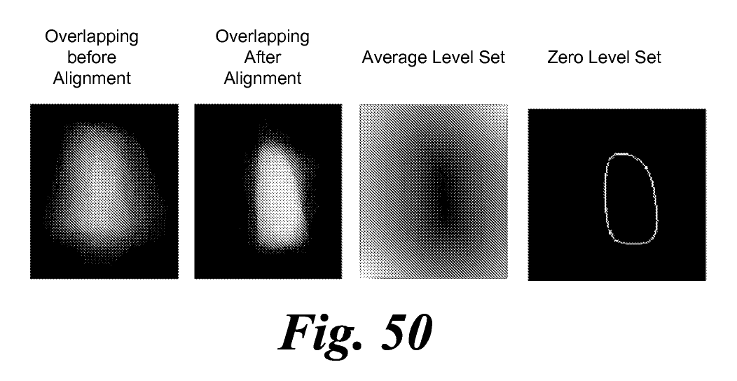

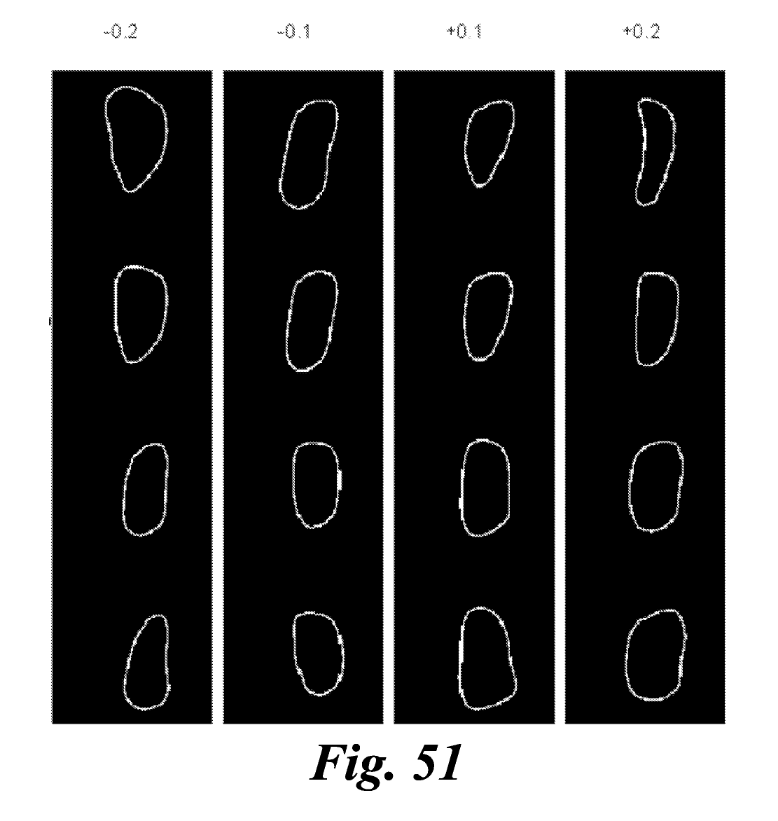

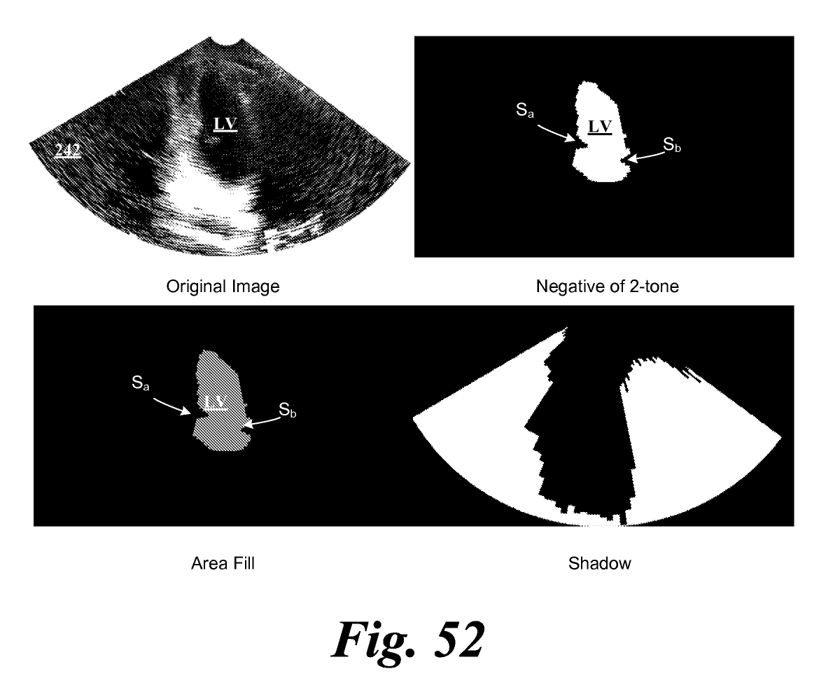

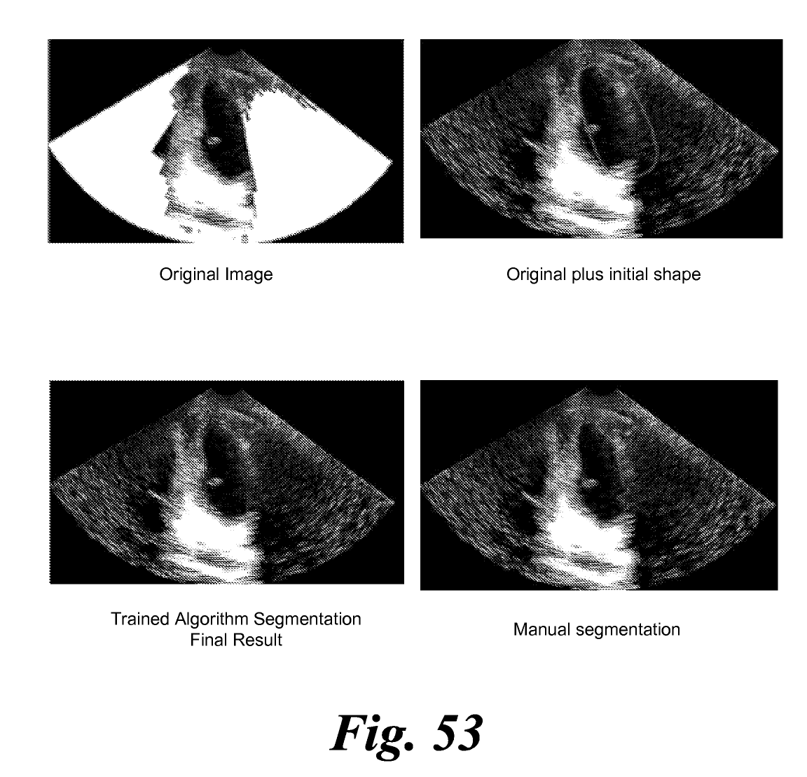

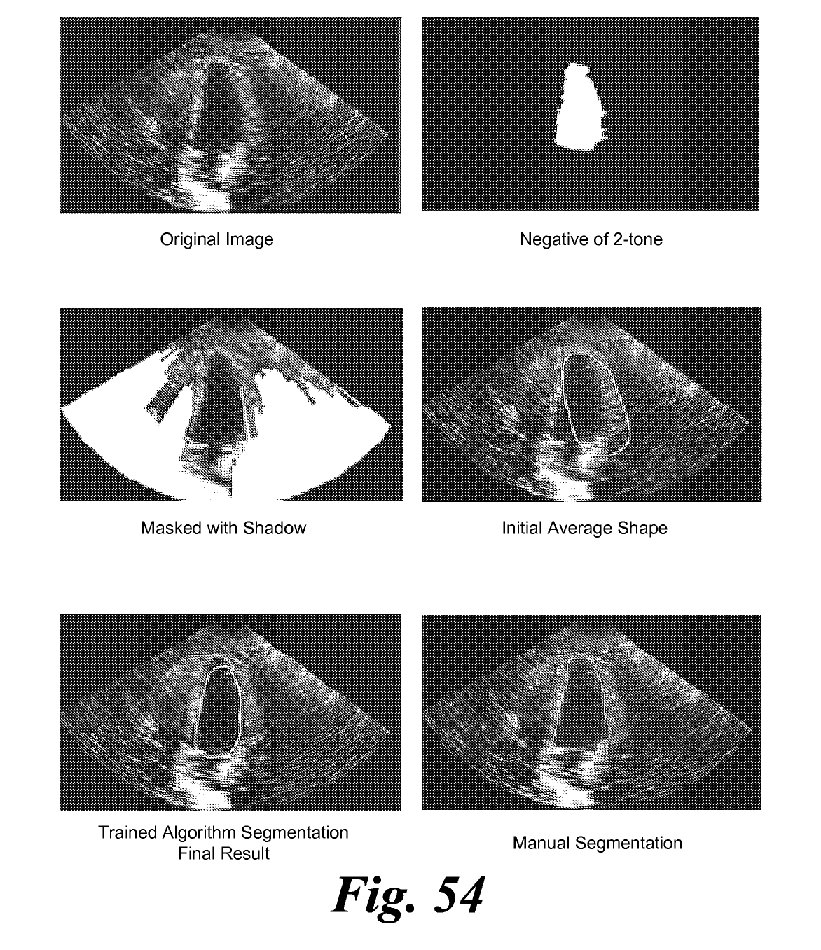

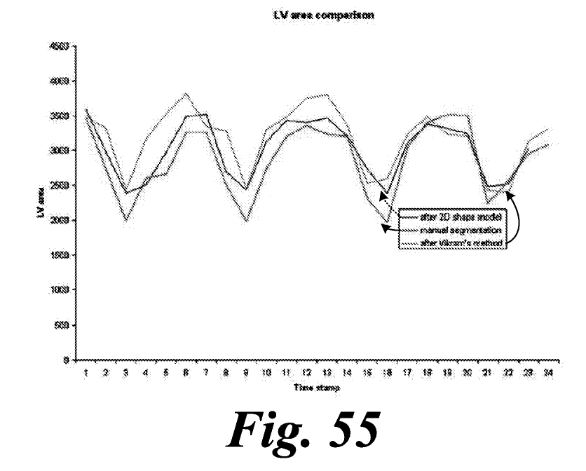

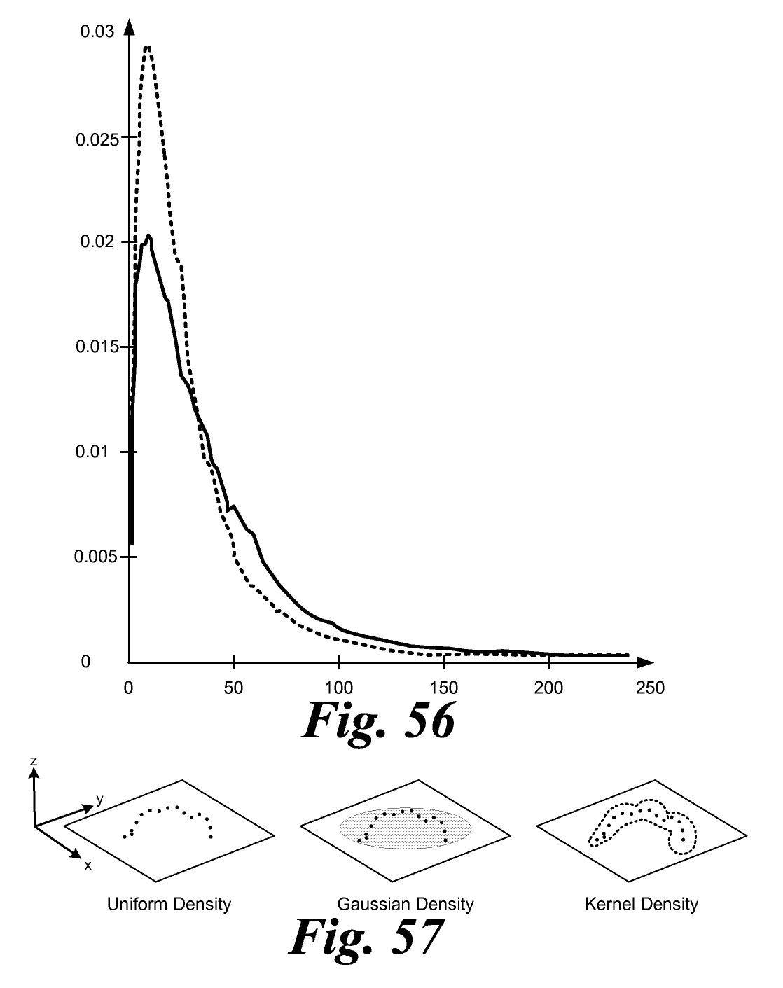

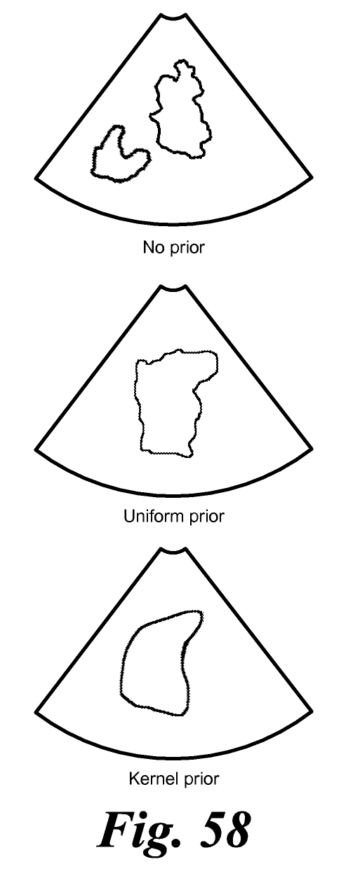

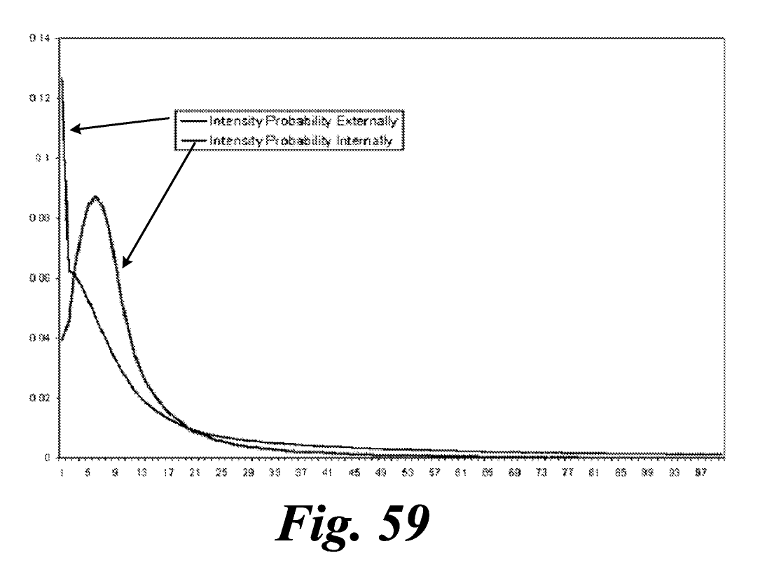

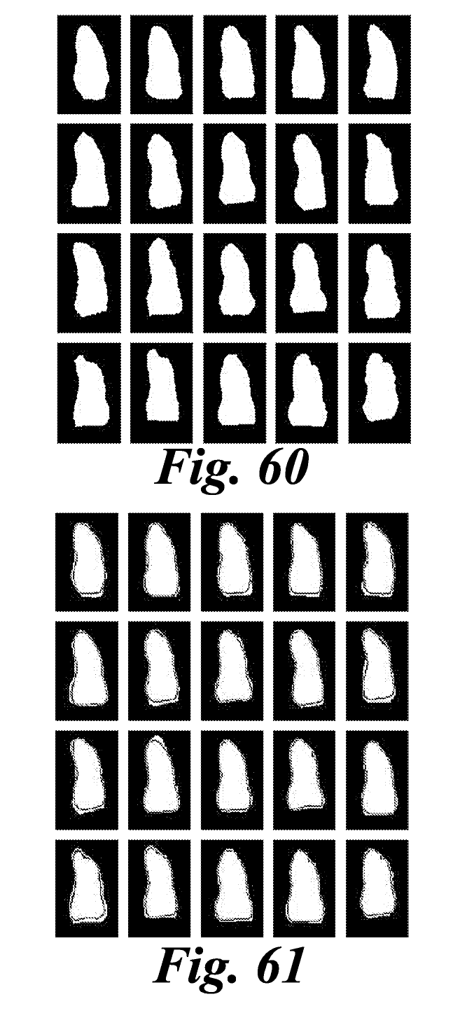

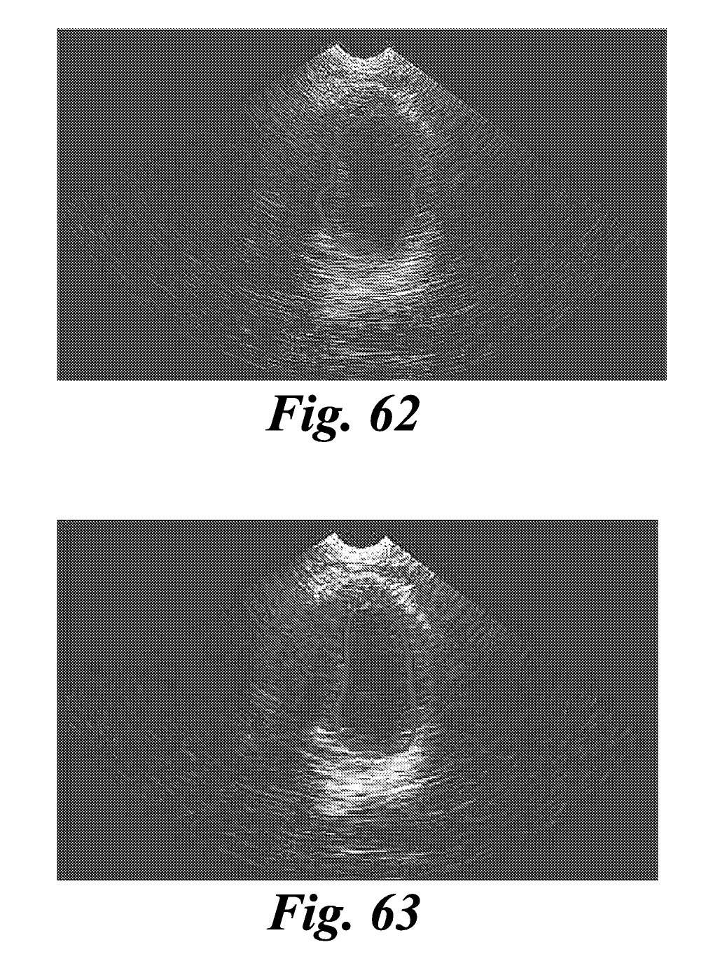

FIG. 13 depicts an alternate embodiment of the cardiac imaging system using an electrocardiograph in communication with a wireless ultrasound transceiver displaying a centered cardiac ROI;

FIG. 14 depicts an alternate embodiment of the cardiac imaging system using an electrocardiograph in communication with a wired connected ultrasound transceiver;

FIG. 15 schematically depicts an alternate embodiment of the cardiac imaging system during Doppler targeting with microphone equipped transceivers 10A-D;

FIG. 16 schematically depicts an alternate embodiment of the cardiac imaging system during Doppler targeting of a transceiver with a speaker equipped electrocardiograph;

FIG. 17 schematically depicts an alternate embodiment of the cardiac imaging system during Doppler targeting of a speaker-less transceiver 10E with a speaker equipped electrocardiograph;

FIG. 18 is a schematic illustration and partial isometric view of a network connected cardio imaging ultrasound system 100 in communication with ultrasound imaging systems 60A-D;

FIG. 19 is a schematic illustration and partial isometric view of an Internet connected cardio imaging ultrasound system 110 in communication with ultrasound imaging systems 60A-D;

FIG. 20 is an algorithm flowchart 200 for the method to measure and determine heart chamber volumes, changes in heart chamber volumes, ICWT and ICWM;

FIG. 21 is an expansion of sonographer-executed sub-algorithm 204 of flowchart in FIG. 20 that utilizes a 2-step enhancement process;

FIG. 22 is an expansion of sonographer-executed sub-algorithm 224 of flowchart in FIG. 20 that utilizes a 3-step enhancement process;

FIG. 23A is an expansion of sub-algorithm 260 of flowchart algorithm depicted in FIG. 20;

FIG. 23B is an expansion of sub-algorithm 300 of flowchart algorithm depicted in FIG. 20 for application to non-database images acquired in process block 280;

FIG. 24 is an expansion of sub-algorithm 280 of flowchart algorithm 200 in FIG. 20;

FIG. 25 is an expansion of sub-algorithm 310 of flowchart algorithm 200 in FIG. 20;

FIG. 26 is an 8-image panel exemplary output of segmenting the left ventricle by processes of sub-algorithm 220;

FIG. 27 presents a scan plane image with ROI of the heart delineated with echoes returning from 3.5 MHz pulsed ultrasound;

FIG. 28 is a schematic of application of snakes processing block of sub-algorithm 220 to an active contour model;

FIG. 29 is a schematic of application of level-set processing block of sub-algorithm 260 of FIG. 23 to an active contour model.

FIG. 30 illustrates a 12-panel outline of a left ventricle determined by an experienced sonographer overlapped before alignment by gradient descent;

FIG. 31 illustrates a 12-panel outline of a left ventricle determined by an experienced sonographer that are overlapped by gradient decent alignment between zero and level set outlines;

FIG. 32 illustrates the procedure for creation of a matrix S of a N₁×N₂ rectangular grid;

FIG. 33 is illustrates a training 12-panel eigenvector image set generated by distance mapping per process block 268 to extract mean eigen shapes;

FIG. 34 illustrates the 12-panel training eigenvector image set wherein ventricle boundary outlines are overlapped;

FIG. 35 illustrated the effects of using different w or k-eigenshapes to control the appearance and newly generated shapes;

FIG. 36 is an image of variation in 3D space affected by changes in 2D measurements over time;

FIG. 37 is a 7-panel phantom training image set compared with a 7-panel aligned set;

FIG. 38 is a phantom training set comprising variations in shapes;

FIG. 39 illustrates the restoration of properly segmented phantom measured structures from an initially compromised image using the aforementioned particular embodiments;

FIG. 40 schematically depicts a particular embodiment to determine shape segmentation of a ROI;

FIG. 41 illustrates an exemplary transthoracic apical view of two heart chambers;

FIG. 42 illustrates other exemplary transthoracic apical views as panel sets associated with different rotational scan plane angles;

FIG. 43 illustrates a left ventricle segmentation from different weight values w applied to a panel of eigenvector shapes;

FIG. 44 illustrates exemplary Left Ventricle segmentations using the trained level-set algorithms;

FIG. 45 is a plot of the level-set automated left ventricle area vs. the sonographer or manually measured area of angle 1003-000 from Table 3;

FIG. 46 is a plot of the level-set automated left ventricle area vs. the sonographer or manually measured area of angle 1003-030 from Table 4;

FIG. 47 is a plot of the level-set automated left ventricle area vs. the sonographer or manually measured area of angle 1003-060 from Table 5;

FIG. 48 is a plot of the level-set automated left ventricle area vs. the sonographer or manually measured area of angle 1003-090 from Table 6;

FIG. 49 illustrates the 3D-rendering of a portion of the Left Ventricle from 30 degree angular view presented from six scan planes obtained at systole and diastole;

FIG. 50 illustrates 4 eigenvector images undergoing different shape variations from a set of varying weight values w applied to the eigenvectors. A total of 16 shape variations are created with w values of −0.2, −0.1, +1, and +2;

FIG. 51 illustrates a series of Left Ventricle images undergoing shape alignment of the 16 eigenvector panel of FIG. 50 using the training sub-algorithm 264 of FIG. 23;

FIG. 52 presents an image result showing boundary artifacts of a left ventricle that arises by employing the estimate shadow regions algorithm 234 of FIG. 22;

FIG. 54 illustrates another panel of exemplary images showing the incremental effects of application of an alternate embodiment of the level-set sub-algorithm 260 of FIG. 23;

FIG. 54 illustrates another panel of exemplary images showing the incremental effects of application of level-set sub-algorithm 260 of FIG. 23;

FIG. 55 presents a graphic of Left Ventricle area determination as a function of 2D segmentation with time (2D+time) between systole and diastole by application of the particular and alternate embodiments of the level set algorithms of FIG. 23;

FIG. 56 illustrates cardiac ultrasound echo histograms of the left ventricle;

FIG. 57 depicts three panels in which schematic representations of a curved shaped eigenvector of a portion of a left ventricle is progressively detected when applied under uniform, Gaussian, and Kernel density pixel intensity distributions;

FIG. 58 depicts segmentation of the left ventricle arising from different a-priori model assumptions;

FIG. 59 is a histogram plot of 20 left ventricle scan planes to determine boundary intensity probability distributions employed for establishing segmentation within training data sets of the left ventricle;

FIG. 60 depicts a panel of aligned training shapes of the left ventricle from the data contained in Table 3;

FIG. 61 depicts the overlaying of the segmented left ventricle to the 20-image panel training set obtained by the application of level set algorithm generated eigen vectors of Table 6;

FIG. 62 depicts application of a non-model segmentation to an image of a subject's left ventricle; and

FIG. 63 depicts application of a kernel-model segmentation to the same image of the subject's left ventricle.

### DETAILED DESCRIPTION OF THE PREFERRED EMBODIMENT

One preferred embodiment includes a three dimensional (3D) ultrasound-based hand-held 3D ultrasound device to acquire at least one 3D data set of a heart in order to measure a change in left ventricle volume at end-diastolic and end-systole time points as determined by an accompanying ECG device. The difference of left ventricle volumes at end-diastolic and end-systole time points is an ultrasound-based ventricular ejection fraction measurement.

A hand-held 3D ultrasound device is used to image a heart. A user places the device over a chest cavity, and initially acquires a 2D image to locate a heart. Once located, a 3D scan is acquired of a heart, preferably at ECG determined time points. A user acquires one or more 3D image data sets as an array of 2D images based upon the signals of an ultrasound echoes reflected from exterior and interior cardiac surfaces for each of an ECG-determined time points. 3D image data sets are stored, preferably in a device and/or transferred to a host computer or network for algorithmic processing of echogenic signals collected by the ultrasound device.

The methods further include a plurality of automated processes optimized to accurately locate, delineate, and measure a change in left ventricle volume. Preferably, this is achieved in a cooperative manner by synchronizing a left ventricle measurements with an ECG device used to acquire and to identify an end-diastolic and end-systole time points in the cardiac cycle. Left ventricle volumes are reconstructed at end-diastole and end-systole time points in the cardiac cycle. A difference between a reconstructed end-diastole and end-systole time points represents a left ventricular ejection fraction. Preferably, an automated process uses a plurality of algorithms in a sequence that includes steps for image enhancement, segmentation, and polishing of ultrasound-based images taken at an ECG determined and identified time points.

A 3D ultrasound device is configured or configurable to acquire 3D image data sets in at least one form or format, but preferably in two or more forms or formats. A first format is a set or collection of one or more two-dimensional scanplanes, one or more, or preferably each, of such scanplanes being separated from another and representing a portion of a heart being scanned.

Registration of Data from Different Viewpoints

An alternate embodiment includes an ultrasound acquisition protocol that calls for data acquisition from one or more different locations, preferably from under the ribs and from between different intercostal spaces. Multiple views maximize the visibility of the left ventricle and enable viewing the heart from two or more different viewpoints. In one preferred embodiment, the system and method aligns and “fuses” the different views of the heart into one consistent view, thereby significantly increasing a signal to noise ratio and minimizing the edge dropouts that make boundary detection difficult.

In a preferred embodiment, image registration technology is used to align these different views of a heart, in some embodiments in a manner similar to how applicants have previously used image registration technology to generate composite fields of view for bladder and other non-cardiac images in applications referenced above. This registration can be performed independently for end-diastolic and end-systolic cones.

An initial transformation between two 3D scancones is conducted to provide an initial alignment of the each 3D scancone's reference system. Data utilized to achieve this initial alignment or transformation is obtained from on board accelerometers that reside in a transceiver 10 (not shown). This initial transformation launches an image-based registration process as described below. An image-based registration algorithm uses mutual information, preferably from one or more images, or another metric to maximize a correlation between different 3D scancones or scanplane arrays. In one embodiment, such registration algorithms are executed during a process of trying to determine a 3D rigid registration process (for example, at 3 rotations and 3 translations) between 3D scancones of data. In alternate embodiments, to account for breathing, a non-rigid transformation is algorithm is applied.

Preferably, once some or all of the data from some or all of the different viewpoints has been registered, and preferably fused, a boundary detection procedure, preferably automatic, is used to permit the visualization of the LV boundary, so as to facilitate calculating the LV volume. In some embodiments it is preferable for all the data to be gathered before boundary detection begins. In other embodiments, processing is done partly in parallel, whereby boundary detection can begin before registration and/or fusing is complete.

One or more of, or preferably each scanplane is formed from one-dimensional ultrasound A-lines within a 2D scanplane. 3D data sets are then represented, preferably as a 3D array of 2D scanplanes. A 3D array of 2D scanplanes is preferably an assembly of scanplanes, and may be assembled into any form of array, but preferably one or more or a combination or sub-combination of any the following: a translational array, a wedge array, or a rotational array.

Alternatively, a 3D ultrasound device is configured to acquire 3D image data sets from one-dimensional ultrasound A-lines distributed in 3D space of a heart to form a 3D scancone of 3D-distributed scanline. In this embodiment, a 3D scancone is not an assembly of 2D scanplanes. In other embodiments, a combination of both: (a) assembled 2D scanplanes; and (b) 3D image data sets from one-dimensional ultrasound A-lines distributed in 3D space of a heart to form a 3D scancone of 3D-distributed scanline is utilized.

A 3D image datasets, either as discrete scanplanes or 3D distributed scanlines, are subjected to image enhancement and analysis processes. The processes are either implemented on a device itself or implemented on a host computer. Alternatively, the processes can also be implemented on a server or other computer to which 3D ultrasound data sets are transferred.

In a preferred image enhancement process, one or more, or preferably each 2D image in a 3D dataset is first enhanced using non-linear filters by an image pre-filtering step. An image pre-filtering step includes an image-smoothing step to reduce image noise followed by an image-sharpening step to obtain maximum contrast between organ wall boundaries. In alternate embodiments, this step is omitted, or preceded by other steps.

A second process includes subjecting a resulting image of a first process to a location method to identify initial edge points between blood fluids and other cardiac structures. A location method preferably automatically determines the leading and trailing regions of wall locations along an A-mode one-dimensional scan line. In alternate embodiments, this step is omitted, or preceded by other steps.

A third process includes subjecting the image of a first process to an intensity-based segmentation process where dark pixels (representing fluid) are automatically separated from bright pixels (representing tissue and other structures). In alternate embodiments, this step is omitted, or preceded by other steps.

In a fourth process, the images resulting from a second and third step are combined to result in a single image representing likely cardiac fluid regions. In alternate embodiments, this step is omitted, or preceded by other steps.

In a fifth process, the combined image is cleaned to make the output image smooth and to remove extraneous structures. In alternate embodiments, this step is omitted, or preceded by other steps.

In a sixth process, boundary line contours are placed on one or more, but preferably each 2D image. Preferably thereafter, the method then calculates the total 3D volume of a left ventricle of a heart. In alternate embodiments, this step is omitted, or preceded by other steps.

In cases in which a heart is either too large to fit in a single 3D array of 2D scanplanes or a single 3D scancone of 3D distributed scanlines, or is otherwise obscured by a view blocking rib, alternate embodiments of the invention allow for acquiring one or more, preferably at least two 3D data sets, and even more preferably four, one or more of, and preferably each 3D data set having at least a partial ultrasonic view of a heart, each partial view obtained from a different anatomical site of a patient.

In one embodiment a 3D array of 2D scanplanes is assembled such that a 3D array presents a composite image of a heart that displays left ventricle regions to provide a basis for calculation of cardiac ejection fractions. In a preferred alternate embodiment, a user acquires 3D data sets in one or more, or preferably multiple sections of the chest region when a patient is being ultrasonically probed. In this multiple section procedure, at least one, but preferably two cones of data are acquired near the midpoint (although other locations are possible) of one or more, but preferably each heart quadrant, preferably at substantially equally spaced (or alternately, uniform, non-uniform or predetermined or known or other) intervals between quadrant centers. Image processing as outlined above is conducted for each quadrant image, segmenting on the darker pixels or voxels associated with the blood fluids. Correcting algorithms are applied to compensate for any quadrant-to-quadrant image cone overlap by registering and fixing one quadrant's image to another. The result is a fixed 3D mosaic image of a heart and the cardiac ejection fractions or regions in a heart from the four separate image cones.

Similarly, in another preferred alternate embodiment, a user acquires one or more 3D image data sets of quarter sections of a heart when a patient is in a lateral position. In this multi-image cone lateral procedure, one or more, but preferably each image cone of data is acquired along a lateral line of substantially equally spaced (or alternately, uniform, or predetermined or known) intervals. One or more, or preferably, each image cone is subjected to the image processing as outlined above, preferably with emphasis given to segmenting on the darker pixels or voxels associated with blood fluid. Scanplanes showing common pixel or voxel overlaps are registered into a common coordinate system along the lateral line. Correcting algorithms are applied to compensate for any image cone overlap along the lateral line. The result is the ability to create and display a fixed 3D mosaic image of a heart and the cardiac ejection fractions or regions in a heart from the four separate image cones. In alternate embodiments fewer or more steps, or alternate sequences are utilized.

In yet other preferred embodiments, at least one, but preferably two 3D scancones of 3D distributed scanlines are acquired at different anatomical sites, image processed, registered and fused into a 3D mosaic image composite. Cardiac ejection fractions are then calculated.

The system and method further optionally and/or alternately provides an automatic method to detect and correct for any contribution non-cardiac obstructions provide to the cardiac ejection fraction. For example, ribs, tumors, growths, fat, or any other obstruction not intended to be measured as part of EF can be detected and corrected for.

A preferred portable embodiment of an ultrasound transceiver of a cardiac ejection fraction measuring system is shown in FIGS. 1-4. A transceiver 10 includes a handle 12 having a trigger 14 and a top button 16, a transceiver housing 18 attached to a handle 12, and a transceiver dome 20. A display 24 for user interaction is attached to a transceiver housing 18 at an end opposite a transceiver dome 20. Housed within a transceiver 10 is a single element transducer (not shown) that converts ultrasound waves to electrical signals. A transceiver 10 is held in position against the body of a patient by a user for image acquisition and signal processing. In a preferred embodiment, a transceiver 10 transmits a radio frequency ultrasound signal at substantially 3.7 MHz to the body and then receives a returning echo signal; however, in alternate embodiments the ultrasound signal can transmit at any radio frequency. To accommodate different patients having a variable range of obesity, a transceiver 10 can be adjusted to transmit a range of probing ultrasound energy from approximately 2 MHz to approximately 10 MHz radio frequencies (or throughout a frequency range), though a particular embodiment utilizes a 3-5 MHz range. A transceiver 10 may commonly acquire 5-10 frames per second, but may range from 1 to approximately 200 frames per second. A transceiver 10, as described below in FIG. 11 below, wirelessly communicates with an ECG device coupled to the patent and includes embedded software to collect and process data. Alternatively, a transceiver 10 may be connected to an ECG device by electrical conduits.

A top button 16 selects for different acquisition volumes. A transceiver is controlled by a microprocessor and software associated with a microprocessor and a digital signal processor of a computer system. As used in this invention, the term “computer system” broadly comprises any microprocessor-based or other computer system capable of executing operating instructions and manipulating data, and is not limited to a traditional desktop or notebook computer. A display 24 presents alphanumeric or graphic data indicating a proper or optimal positioning of a transceiver 10 for initiating a series of scans. A transceiver 10 is configured to initiate a series of scans to obtain and present 3D images as either a 3D array of 2D scanplanes or as a single 3D scancone of 3D distributed scanlines. A suitable transceiver is a transceiver 10 referred to in the FIGURES. In alternate embodiments, a two- or three-dimensional image of a scan plane may be presented in a display 24.

Although a preferred ultrasound transceiver is described above, other transceivers may also be used. For example, a transceiver need not be battery-operated or otherwise portable, need not have a top-mounted display 24, and may include many other features or differences. A display 24 may be a liquid crystal display (LCD), a light emitting diode (LED), a cathode ray tube (CRT), or any suitable display capable of presenting alphanumeric data or graphic images.

FIG. 2A is a photograph of a hand-held transceiver 10 for scanning in a chest region of a patient. In an inset figure, a transceiver 10 is positioned over a patient's chest by a user holding a handle 12 to place a transceiver housing 18 against a patient's chest. A sonic gel pad 19 is placed on a patient's chest, and a transceiver dome 20 is pressed into a sonic gel pad 19. A sonic gel pad 19 is an acoustic medium that efficiently transfers an ultrasonic radiation into a patient by reducing the attenuation that might otherwise significantly occur were there to be a significant air gap between a transceiver dome 20 and a surface of a patient. A top button 16 is centrally located on a handle 12. Once optimally positioned over an abdomen for scanning, a transceiver 10 transmits an ultrasound signal at substantially 3.7 MHz into a heart; however, in alternate embodiments the ultrasound signal can transmit at any radio frequency. A transceiver 10 receives a return ultrasound echo signal emanating from a heart and presents it on a display 24.

Further FIG. 2A depicts a transceiver housing 18 is positioned such that a dome 20, whose apex is at or near a bottom of a heart, an apical view may be taken from spaces between lower ribs near a patient's side and pointed towards a patient's neck.

FIG. 2B is a perspective view of a hand-held transceiver device sitting in a communication cradle 42. A transceiver 10 sits in a communication cradle 42 via a handle 12. This cradle can be connected to a standard USB port of any personal computer or other signal conveyance means, enabling all data on a device to be transferred to a computer and enabling new programs to be transferred into a device from a computer. Further a heart is depicted in a cross hatched pattern beneath the rib cage of a patient FIG. 3 is a perspective view of a cardiac ejection fraction measuring system 5A. A system 5A includes a transceiver 10 cradled in a cradle 42 that is in signal communication with a computer 52. A transceiver 10 sits in a communication cradle 42 via a handle 12. This cradle can be connected to a standard USB port of any personal computer 52, enabling all data on a transceiver 10 to be transferred to a computer for analysis and determination of cardiac ejection fraction. However in an alternate embodiment the cradle may be connect by any means of signal transfer.

FIG. 4 depicts an alternate embodiment of a cardiac ejection fraction measuring system 5B in a schematic view. A system 5B includes a plurality of systems 5A in signal communication with a server 56. As illustrated each transceiver 10 is in signal connection with a server 56 through connections via a plurality of computers 52. FIG. 3, by example, depicts each transceiver 10 being used to send probing ultrasound radiation to a heart of a patient and to subsequently retrieve ultrasound echoes returning from a heart, convert ultrasound echoes into digital echo signals, store digital echo signals, and process digital echo signals by algorithms of an invention. A user holds a transceiver 10 by a handle 12 to send probing ultrasound signals and to receive incoming ultrasound echoes. A transceiver 10 is placed in a communication cradle 42 that is in signal communication with a computer 52, and operates as a cardiac ejection fraction measuring system. Two cardiac ejection fraction-measuring systems are depicted as representative though fewer or more systems may be used. As used in this invention, a “server” can be any computer software or hardware that responds to requests or issues commands to or from a client. Likewise, a server may be accessible by one or more client computers via the Internet, or may be in communication over a LAN or other network. A server 56 includes executable software that has instructions to reconstruct data, detect left ventricle boundaries, measure volume, and calculate change in volume or percentage change in volume. In alternate embodiments fewer or more steps, or alternate sequences are utilized.

One or more, or preferably each, cardiac ejection fraction measuring systems includes a transceiver 10 for acquiring data from a patient. A transceiver 10 is placed in a cradle 42 to establish signal communication with a computer 52. Signal communication as illustrated by a wired connection from a cradle 42 to a computer 52. Signal communication between a transceiver 10 and a computer 52 may also be by wireless means, for example, infrared signals or radio frequency signals. A wireless means of signal communication may occur between a cradle 42 and a computer 52, a transceiver 10 and a computer 52, or a transceiver 10 and a cradle 42. In alternate embodiments fewer or more steps, or alternate sequences are utilized.

A preferred first embodiment of a cardiac ejection fraction measuring system includes one or more, or preferably each, transceiver 10 being separately used on a patient and sending signals proportionate to the received and acquired ultrasound echoes to a computer 52 for storage. Residing in one or more, or preferably each, computer 52 are imaging programs having instructions to prepare and analyze a plurality of one dimensional (1D) images from stored signals and transforms a plurality of 1D images into a plurality of 2D scanplanes. Imaging programs also present 3D renderings from a plurality of 2D scanplanes. Also residing in one or more, or preferably each, computer 52 are instructions to perform additional ultrasound image enhancement procedures, including instructions to implement image processing algorithms. In alternate embodiments fewer or more steps, or alternate sequences are utilized.

A preferred second embodiment of a cardiac ejection fraction measuring system is similar to a first embodiment, but imaging programs and instructions to perform additional ultrasound enhancement procedures are located on a server 56. One or more, or preferably each, computer 52 from one or more, or preferably each, cardiac ejection fraction measuring system receives acquired signals from a transceiver 10 via a cradle 42 and stores signals in memory of a computer 52. A computer 52 subsequently retrieves imaging programs and instructions to perform additional ultrasound enhancement procedures from a server 56. Thereafter, one or more, or preferably each, computer 52 prepares 1D images, 2D images, 3D renderings, and enhanced images from retrieved imaging and ultrasound enhancement procedures. Results from data analysis procedures are sent to a server 56 for storage. In alternate embodiments fewer or more steps, or alternate sequences are utilized.

A preferred third embodiment of a cardiac ejection fraction measuring system is similar to the first and second embodiment, but imaging programs and instructions to perform additional ultrasound enhancement procedures are located on a server 56 and executed on a server 56. One or more, or preferably each, computer 52 from one or more, or preferably each, cardiac ejection fraction measuring system receives acquired signals from a transceiver 10 and via a cradle 42 sends the acquired signals in the memory of a computer 52. A computer 52 subsequently sends a stored signal to a server 56. In a server 56, imaging programs and instructions to perform additional ultrasound enhancement procedures are executed to prepare the 1D images, 2D images, 3D renderings, and enhanced images from a server's 56 stored signals. Results from data analysis procedures are kept on a server 56, or alternatively, sent to a computer 52. In alternate embodiments fewer or more steps, or alternate sequences are utilized.

FIG. 5 is another embodiment of a cardiac ejection fraction measuring system 5C presented in schematic view. The system 5C includes a plurality of cardiac ejection fraction measuring systems 5A connected to a server 56 over the Internet or other network 64. FIG. 4 represents any of a first, second, or third embodiments of an invention advantageously deployed to other servers and computer systems through connections via a network.

FIG. 6A a graphical representation of a plurality of scan lines forming a single scan plane. FIG. 6A illustrates how ultrasound signals are used to make analyzable images, more specifically how a series of one-dimensional (1D) scanlines are used to produce a two-dimensional (2D) image. The 1D and 2D operational aspects of the single element transducer housed in the transceiver 10 is seen as it rotates mechanically about an tilt angle φ. A scanline 214 of length r migrates between a first limiting position 218 and a second limiting position 222 as determined by the value of the tilt angle φ, creating a fan-like 2D scanplane 210. In one preferred form, the transceiver 10 operates substantially at 3.7 MHz frequency and creates an approximately 18 cm deep scan line 214 and migrates within the tilt angle φhaving an angle intervals of approximately 0.027 radians. However, in alternate embodiments the ultrasound signal can transmit at any radio frequency, the scan line can have any length (r), and angle intervals of any operable size. In a preferred embodiment a first motor tilts the transducer approximately 60° clockwise and then counterclockwise forming the fan-like 2D scanplane presenting an approximate 120° 2D sector image. However in alternative embodiments the motor may tilt at any degree measurement and either clockwise or counterclockwise. A plurality of scanlines, one or more, or preferably each, scanline substantially equivalent to scanline 214 is recorded, between the first limiting position 218 and the second limiting position 222 formed by the unique tilt angle φ. In a preferred embodiment a plurality of scanlines between two extremes forms a scanplane 210. In the preferred embodiment, one or more, or preferably each, scanplane contains 77 scan lines, although the number of lines can vary within the scope of this invention. The tilt angle φ sweeps through angles approximately between −60° and +60° for a total arc of approximately 120°.

FIG. 6B is a graphical representation of a plurality of scanplanes forming a three-dimensional array (3D) 240 having a substantially conic shape. FIG. 6B illustrates how a 3D rendering is obtained from a plurality of 2D scanplanes. Within one or more, or preferably each, scanplane 210 are a plurality of scanlines, one or more, or preferably each, scanline equivalent to a scanline 214 and sharing a common rotational angle θ. In the preferred embodiment, one or more, or preferably each, scanplane contains 77 scan lines, although the number of lines can vary within the scope of this invention. One or more, or preferably each, 2D sector image scanplane 210 with tilt angle φ and length r (equivalent to a scanline 214) collectively forms a 3D conic array 240 with rotation angle θ. After gathering a 2D sector image, a second motor rotates a transducer between 3.75° or 7.5° to gather the next 120° sector image. This process is repeated until a transducer is rotated through 180°, resulting in a cone-shaped 3D conic array 240 data set with 24 planes rotationally assembled in the preferred embodiment. A conic array could have fewer or more planes rotationally assembled. For example, preferred alternate embodiments of a conic array could include at least two scanplanes, or a range of scanplanes from 2 to 48 scanplanes. The upper range of the scanplanes can be greater than 48 scanplanes. The tilt angle φ indicates the tilt of a scanline from the centerline in 2D sector image, and the rotation angle θ, identifies the particular rotation plane the sector image lies in. Therefore, any point in this 3D data set can be isolated using coordinates expressed as three parameters, P(r, φ, θ).

As scanlines are transmitted and received, the returning echoes are interpreted as analog electrical signals by a transducer, converted to digital signals by an analog-to-digital converter, and conveyed to the digital signal processor of a computer system for storage and analysis to determine the locations of the cardiac external and internal walls or septa. A computer system is representationally depicted in FIGS. 3 and 4 and includes a microprocessor, random access memory (RAM), or other memory for storing processing instructions and data generated by a transceiver 10.

FIG. 6C is a graphical representation of a plurality of 3D-distributed scanlines emanating from a transceiver 10 forming a scancone 300. A scancone 300 is formed by a plurality of 3D distributed scanlines that comprises a plurality of internal and peripheral scanlines. Scanlines are one-dimensional ultrasound A-lines that emanate from a transceiver 10 at different coordinate directions, that taken as an aggregate, from a conic shape. 3D-distributed A-lines (scanlines) are not necessarily confined within a scanplane, but instead are directed to sweep throughout the internal and along the periphery of a scancone 300. A 3D-distributed scanlines not only would occupy a given scanplane in a 3D array of 2D scanplanes, but also the inter-scanplane spaces, from a conic axis to and including a conic periphery. A transceiver 10 shows the same illustrated features from FIG. 1, but is configured to distribute ultrasound A-lines throughout 3D space in different coordinate directions to form a scancone 300.

Internal scanlines are represented by scanlines 312A-C. The number and location of internal scanlines emanating from a transceiver 10 is a number of internal scanlines needed to be distributed within a scancone 300, at different positional coordinates, to sufficiently visualize structures or images within a scancone 300. Internal scanlines are not peripheral scanlines. Peripheral scanlines are represented by scanlines 314A-F and occupy a conic periphery, thus representing the peripheral limits of a scancone 300.

FIG. 7 is a cross sectional schematic of a heart. The four chambered heart includes the right ventricle RV, the right atrium RA, the left ventricle LV, the left atrium LA, an inter ventricular septum IVS, a pulmonary valve PVa, a pulmonary vein PV, a right atrium ventricular valve R. AV, a left atrium ventricular valve L. AV, a superior vena cava SVC, an inferior vena cava IVC, a pulmonary trunk PT, a pulmonary artery PA, and aorta. The arrows indicate direction of blood flow. The difference between the end diastolic volume and the end systolic volume of the left ventricle is defined to be the stroke volume and corresponds to the amount of blood pumped into the aorta during one cardiac beat. The ratio of the stroke volume to the end diastolic volume is the ejection fraction. This ejection fraction represents the contractility of the heart muscle cells. Making ultrasound-based volume measurements in the left ventricle at ECG-determined end diastolic and end systolic time points provide the basis to calculate the cardiac ejection fraction.

FIG. 8 is a two-component graph of a heart cycle diagram. The diagram points out two landmark volume measurements at an end diastolic and an systolic time points in a left ventricle. A volume difference at these two time points is a stroke volume or ejection fraction of blood being pumped into an aorta.

FIG. 9 is a schematic depiction of a scanplane overlaid upon a cross section of a heart. Scanlines 214 that comprise a scanplane 210 are shown emanating from a dome 20 of a transceiver 10 and penetrate towards and through the cavities, blood vessels, and septa of a heart.

FIG. 10A is a schematic depiction of an ejection fraction measuring system in operation on a patient. An ejection fraction measuring system 350 includes a transceiver 10 and an electrocardiograph ECG 370 equipped with a transmitter. Connected to an ECG 370 are probes 372, 374, and 376 that are placed upon a subject to make a cardiac ejection fraction determination. An ECG 370 has lead connections to the electric potential probes 372, 374, and 376 to receive ECG signals. A probe 372 is located on a right shoulder of the subject, a probe 374 is located on a left shoulder, and a probe 376 is located a lower leg, here depicted as a left lower leg. Instead of a 3-lead ECG as shown for an ECG 370, alternatively, a 2-lead ECG may be configured with probes placed on a left and right shoulder, or a right shoulder and a left abdominal side of the subject. Also in an alternate embodiment any number of leads for an ECG may be used. In alternate embodiments fewer or more steps, or alternate sequences are utilized.

FIG. 10B is a pair of ECG plots from an ECG 370 of FIG. 10A. A QRS plot is shown for electric potential and a ventricular action potential plot having a 0.3 second time base is shown.

FIG. 11 is a schematic depiction and expands the details of the particular embodiment of an ejection fraction measuring system 350. Electric potential signals from probes 372, 374, and 376 are conveyed to transistor 370A and processed by a microprocessor 370B. A microprocessor 370B identifies P-waves and T-waves and a QRS complex of an ECG signal. A microprocessor 370B also generates a dual-tone-multi-frequency (DTMF) signal that uniquely identifies 3 components of an ECG signal and the blank interval time that occurs between 3 components of a signal. Since systole generally takes 0.3 seconds, the duration of a burst is sufficiently short that a blank interval time is communicated for at least 0.15 seconds during systole. A DTMF signal is transmitted from an antenna 370D using short-range electromagnetic waves 390. A transmitter circuit 370 may be battery powered and consist of a coil with a ferrite core to generate short-range electromagnetic fields, commonly less than 12 inches. In alternate embodiments fewer or more steps, or alternate sequences are utilized.

Electromagnetic waves 390 having DTMF signals identifying the QRS-complex and the P-waves and T-wave components of an ECG signal is received by radio-receiver circuit 380 is located within a transceiver 10. The radio receiver circuit 380 receives the radio-transmitted waves 390 from the antenna 370D of an ECG 370 transmitted via antenna 380D wherein a signal is induced. The induced signal is demodulated in demodulator 380A and processed by microprocessor 380B. In alternate embodiments fewer or more steps, or alternate sequences are utilized.

An overview of the how a system is used is described as follows. One format for collecting data is to tilt a transducer through an arc to collect a plane of scan lines. A plane of data collection is then rotated through a small angle before a transducer is tilted to collect another plane of data. This process would continue until an entire 3-dimensional cone of data may be collected. Alternatively, a transducer may be moved in a manner such that individual scan lines are transmitted and received and reconstructed into a 3-dimensional cone volume without first generating a plane of data and then rotating a plane of data collection. In alternate embodiments fewer or more steps, or alternate sequences are utilized.

To scan a patient, the leads of the ECG are connected to the appropriate locations on the patient's body. The ECG transmitter is turned on such that it is communicating the ECG signal to the transceiver. In alternate embodiments fewer or more steps, or alternate sequences are utilized.

For a first set of data collection, a transceiver 10 is placed just below a patients ribs slightly to a patient's left of a patient's mid-line. A transceiver 10 is pressed firmly into an abdomen and angled towards a patient's head such that a heart is contained within an ultrasound data cone. After a user hears a heart beat from a transceiver 10, a user initiates data collection. In alternate embodiments fewer or more steps, or alternate sequences are utilized.

A top button 16 of a transceiver 10 is pressed to initiate data collection. Data collection continues until a sufficient amount of ultrasound and ECG signal are acquired to re-construct a volumetric data for a heart at an end-diastole and end-systole positions within the cardiac signal. A motion sensor (not shown) in a transceiver 10 detects whether or not a patient breaths and should therefore ignore the ultrasound data being collected at the time due to errors in registering the 3-dimensional scan lines with each other. A tone instructs a user that ultrasound data is complete. In alternate embodiments fewer or more steps, or alternate sequences are utilized.

After data is collected in this position, the device's display instructs a user to collect data from the intercostal spaces. A user moves the device such that it sits between the ribs and a user will re-initiate data collection by pressing the scan button. A motion sensor detects whether or not a patient is breathing and therefore whether or not data being collected is valid. Data collection continues until the 3-dimensional ultrasound volume can be reconstructed for the end-diastole and end-systole time points in the cardiac cycle. A tone instructs a user that ultrasound data collection is complete. In alternate embodiments fewer or more steps, or alternate sequences are utilized.

A user turns off an ECG device and disconnects one or more leads from a patient. A user would place a transceiver 10 in a cradle 42 that communicates both an ECG and ultrasound data to a computer 52 where data is analyzed and an ejection fraction calculated. Alternatively, data may be analyzed on a server 56 or other computers via the Internet 64. Methods for analyzing this data are described in detail in following sections. In alternate embodiments fewer or more steps, or alternate sequences are utilized.

A protocol for collection of ultrasound from a user's perspective has just been described. An implementation of the data collection from the hardware perspective can occur in two manners: using an ECG signal to gate data collection, and recording an ECG signal with ultrasound data and allow analysis software to re-construct the data volumes at an end-diastole and end-systole time points in a cardiac cycle.

Adjustments to the methods described above allow for data collection to be accomplished via an ECG-gated data acquisition mode, and an ECG-Annotated data acquisition with reconstruction mode. In the ECG-gated data acquisition, a given subject's cardiac cycle is determined in advance and an end-systole and end-diastole time points are predicted before a collection of scanplane data. An ECG-gated method has the benefit of limiting a subject's exposure to ultrasound energy to a minimum in that An ECG-gated method only requires a minimum set of ultrasound data because an end-systole and end-diastole time points are determined in advance of making acquiring ultrasound measures. In the ECG-Annotated data acquisition with reconstruction mode, phase lock loop (PLL) predictor software is not employed and there is no analysis for lock, error (epsilon), and state for ascertaining the end-systole and end-diastole ultrasound measurement time points. Instead, an ECG-annotated method requires collecting continuous ultrasound readings to then reconstruct after taking the ultrasound measurements when an end-systole and end-diastole time points are likely to have occurred.

Method 1: ECG Gated Data Acquisition

If the ultrasound data collection is to be gated by an ECG signal, software in a transceiver 10 monitors an ECG signal and predicts appropriate time points for collecting planes of data, such as end-systole and end-diastole time points.

A DTMF signal transmitted by an ECG transmitter is received by an antenna in a transceiver 10. A signal is demodulated and enters a software-based phase lock loop (PLL) predictor that analyzes an ECG signal. An analyzed signal has three outputs: lock, error (epsilon), and state.

A transceiver 10 collects a plane of ultrasound at a time indicated by a predictor. Preferred time points indicated by the predictor are end-systole and end-diastole time points. If an error signal for that plane of data is too large, then a plane is ignored. A predictor updates timing for data collection and a plane collected in the next cardiac cycle.

Once data has been successfully collected for a plane at end-diastole and end-systole time points, a plane of data collection is rotated and a next plane of data may be collected in a similar manner.

A benefit of gated data acquisition is that a minimal set of ultrasound data needs to be collected, limiting a patient to exposure to ultrasound energy. End-systolic and end-diastolic volumes would not need to be re-constructed from a large data set.

A cardiac cycle can vary from beat to beat due to a number of factors. A gated acquisition may take considerable time to complete particularly if a patient is unable to hold their breath.

In alternate embodiments, the above steps and/or subsets may be omitted, or preceded by other steps.

Method 2: ECG Annotated Data Acquisition with Reconstruction

In an alternate method for data collection, ultrasound data collection would be continuous, as would collection of an ECG signal. Collection would occur for up to 1 minute or longer as needed such that a sufficient amount of data is available for re-constructing the volumetric data at end-diastolic and end-systolic time points in the cardiac cycle.

This implementation does not require software PLL to predict a cardiac cycle and control ultrasound data collection, although it does require a larger amount of data.

Both ECG-gated and ECG-annotated methods described above can be made with multiple 3D scancone measurements to insure a sufficiently completed image of a heart is obtained.

FIG. 12 shows a block diagram overview of an image enhancement, segmentation, and polishing algorithms of a cardiac ejection fraction measuring system. An enhancement, segmentation, and polishing algorithm is applied to one or more, or preferably each, scanplane 210 or to an entire 3D conic array 240 to automatically obtain blood fluid and ventricle regions. For scanplanes substantially equivalent (including or alternatively uniform, or predetermined, or known) to scanplane 210, an algorithm may be expressed in two-dimensional terms and use formulas to convert scanplane pixels (picture elements) into area units. For scan cones substantially equivalent to a 3D conic array 240, algorithms are expressed in three-dimensional terms and use formulas to convert voxels (volume elements) into volume units.

Algorithms expressed in 2D terms are used during a targeting phase where the operator trans-abdominally positions and repositions a transceiver 10 to obtain real-time feedback about a left ventricular area in one or more, or preferably each, scanplane. Algorithms expressed in 3D terms are used to obtain a total cardiac ejection fraction computed from voxels contained within calculated left ventricular regions in a 3D conic array 240.

FIG. 12 represents an overview of a preferred method of the invention and includes a sequence of algorithms, many of which have sub-algorithms described in more specific detail in U.S. patent application Ser. No. 11/119,355 filed Apr. 29, 2005, filed, U.S. provisional patent application Ser. No. 60/566,127 filed Apr. 30, 2004, U.S. patent application Ser. No. 10/701,955 filed Nov. 5, 2003, U.S. patent application Ser. No. 10/443,126 filed May 20, 2003, U.S. patent application Ser. No. 11/061,867 filed Feb. 17, 2005, U.S. provisional patent application Ser. No. 60/545,576, filed Feb. 17, 2004, and U.S. patent application Ser. No. 10/633,186 filed Jul. 31, 2003, herein incorporated by reference as described above in the priority claim.

FIG. 12 begins with inputting data of an unprocessed image at step 410. After unprocessed image data 410 is entered (e.g., read from memory, scanned, or otherwise acquired), it is automatically subjected to an image enhancement algorithm 418 that reduces noise in data (including speckle noise) using one or more equations while preserving salient edges on an image using one or more additional equations. Next, enhanced images are segmented by two different methods whose results are eventually combined. A first segmentation method applies an intensity-based segmentation algorithm 422 for myocardium detection that determines pixels that are potentially tissue pixels based on their intensities. A second segmentation method applies an edge-based segmentation algorithm 438 for blood region detection that relies on detecting the blood fluids and tissue interfaces. Images obtained by a first segmentation algorithm 422 and images obtained by a second segmentation algorithm 438 are brought together via a combination algorithm 442 to eventually provide a left ventricle delineation in a substantially segmented image that shows fluid regions and cardiac cavities of a heart, including an atria and ventricles. A segmented image obtained from a combination algorithm 442 is assisted with a user manual seed point 440 to help start an identification of a left ventricle should a manual input be necessary. Finally an area or a volume of a segmented left ventricle region-of-interest is computed 484 by multiplying pixels by a first resolution factor to obtain area, or voxels by a second resolution factor to obtain volume. For example, for pixels having a size of 0.8 mm by 0.8 mm, a first resolution or conversion factor for pixel area is equivalent to 0.64 mm², and a second resolution or conversion factor for voxel volume is equivalent to 0.512 mm³. Different unit lengths for pixels and voxels may be assigned, with a proportional change in pixel area and voxel volume conversion factors.

The enhancement, segmentation and polishing algorithms depicted in FIG. 12 for measuring blood region fluid areas or volumes are not limited to scanplanes assembled into rotational arrays equivalent to a 3D conic array 240. As additional examples, enhancement, segmentation and polishing algorithms depicted in FIG. 12 apply to translation arrays and wedge arrays. Translation arrays are substantially rectilinear image plane slices from incrementally repositioned ultrasound transceivers that are configured to acquire ultrasound rectilinear scanplanes separated by regular or irregular rectilinear spaces. The translation arrays can be made from transceivers configured to advance incrementally, or may be hand-positioned incrementally by an operator. An operator obtains a wedge array from ultrasound transceivers configured to acquire wedge-shaped scanplanes separated by regular or irregular angular spaces, and either mechanistically advanced or hand-tilted incrementally. Any number of scanplanes can be either translationally assembled or wedge-assembled ranges, but preferably in ranges greater than two scanplanes.

Other preferred embodiments of the enhancement, segmentation and polishing algorithms depicted in FIG. 12 may be applied to images formed by line arrays, either spiral distributed or reconstructed random-lines. Line arrays are defined using points identified by coordinates expressed by the three parameters, P(r, φ, θ), where values or r, φ, and θ can vary.

Enhancement, segmentation and calculation algorithms depicted in FIG. 12 are not limited to ultrasound applications but may be employed in other imaging technologies utilizing scanplane arrays or individual scanplanes. For example, biological-based and non-biological-based images acquired using infrared, visible light, ultraviolet light, microwave, x-ray computed tomography, magnetic resonance, gamma rays, and positron emission are images suitable for algorithms depicted in FIG. 12. Furthermore, algorithms depicted in FIG. 12 can be applied to facsimile transmitted images and documents.

Once Intensity-Based myocardium detection 422 and Edge-Based Segmentation 438 for blood region detection is completed, both segmentation methods use a combining step that combines the results of intensity-based segmentation 422 step and an edge-based segmentation 438 step using an AND Operator of Images 442 in order to delineate chambers of a heart, in particular a left ventricle. An AND Operator of Images 442 is achieved by a pixel-wise Boolean AND operator 442 for left ventricle delineation step to produce a segmented image by computing the pixel intersection of two images. A Boolean AND operation 442 represents pixels as binary numbers and a corresponding assignment of an assigned intersection value as a binary number 1 or 0 by the combination of any two pixels. For example, consider any two pixels, say pixel_{A} and pixel_{B}, which can have a 1 or 0 as assigned values. If pixel_{A}'s value is 1, and pixel_{B}'s value is 1, the assigned intersection value of pixel_{A} and pixel_{B} is 1. If the binary value of pixel_{A} and pixel_{B} are both 0, or if either pixel_{A} or pixel_{B} is 0, then the assigned intersection value of pixel_{A} and pixel_{B} is 0. The Boolean AND operation 442 for left ventricle delineation takes a binary number of any two digital images as input, and outputs a third image with pixel values made equivalent to an intersection of the two input images.

After contours on all images have been delineated, a volume of the segmented structure is computed. Two specific techniques for doing so are disclosed in detail in U.S. Pat. No. 5,235,985 to McMorrow et al, herein incorporated by reference. This patent provides detailed explanations for non-invasively transmitting, receiving and processing ultrasound for calculating volumes of anatomical structures.

In alternate embodiments, the above steps and/or subsets may be omitted, or preceded by other steps.

Automated Boundary Detection

Once 3D left-ventricular data is available, the next step to calculate an ejection fraction is a detection of left ventricular boundaries on one or more, or preferably each, image to enable a calculation of an end-diastolic LV volume and an end-systolic LV volume.

Particular embodiments for ultrasound image segmentation include adaptations of the bladder segmentation method and the amniotic fluid segmentation methods are so applied for ventricular segmentation and determination of the cardiac ejection fraction are herein incorporated by references in aforementioned references cited in the priority claim.

A first step is to apply image enhancement using heat and shock filter technology. This step ensures that a noise and a speckle are reduced in an image while the salient edges are still preserved.

A next step is to determine the points representing the edges between blood and myocardial regions since blood is relatively anechoic compared to the myocardium. An image edge detector such as a first or a second spatial derivative method is used.

In parallel, image pixels corresponding to the cardiac blood region on an image are identified. These regions are typically darker than pixels corresponding to tissue regions on an image and also these regions have very a very different texture compared to a tissue region. Both echogenicity and texture information is used to find blood regions using an automatic thresholding or a clustering approach.

After determining all low level features, edges and region pixels, as above, a next step in a segmentation algorithm might be to combine this low level information along with any manual input to delineate left ventricular boundaries in 3D. Manual seed point at process 440 in some cases may be necessary to ensure that an algorithm detects a left ventricle instead of any other chambers of a heart. This manual input might be in the form of a single seed point inside a left ventricle specified by a user.

From the seed point specified by a user, a 3D level-set-based region-growing algorithm or a 3D snake algorithm may be used to delineate a left ventricle such that boundaries of this region are delimited by edges found in a second step and pixels contained inside a region consist of pixels determined as blood pixels found in a third step.

Another method for 3D LV delineation could be based on an edge linking approach. Here edges found in a second step are linked together via a dynamic programming method which finds a minimum cost path between two points. A cost of a boundary can be defined based on its distance from edge points and also whether a boundary encloses blood regions determined in a third step.

In alternate embodiments, the above steps and/or subsets may be omitted, or preceded by other steps

Multiple Image Cone Acquisition and Image Processing Procedures:

In some embodiments, multiple cones of data acquired at multiple anatomical sampling sites may be advantageous. For example, in some instances, a heart may be too large to completely fit in one cone of data or a transceiver 10 has to be repositioned between the subject's ribs to see a region of a heart more clearly. Thus, under some circumstances, a transceiver 10 is moved to different anatomical locations of a patient to obtain different 3D views of a heart from one or more, or preferably each, measurement or transceiver location.

Obtaining multiple 3D views may be especially needed when a heart is otherwise obscured. In such cases, multiple data cones can be sampled from different anatomical sites at known intervals and then combined into a composite image mosaic to present a large heart in one, continuous image. In order to make a composite image mosaic that is anatomically accurate without duplicating anatomical regions mutually viewed by adjacent data cones, ordinarily it is advantageous to obtain images from adjacent data cones and then register and subsequently fuse them together. In a preferred embodiment, to acquire and process multiple 3D data sets or images cones, at least two 3D image cones are generally preferred, with one image cone defined as fixed, and another image cone defined as moving.

3D image cones obtained from one or more, or preferably each, anatomical site may be in the form of 3D arrays of 2D scanplanes, similar to a 3D conic array 240. Furthermore, a 3D image cone may be in the form of a wedge or a translational array of 2D scanplanes. Alternatively, a 3D image cone obtained from one or more, or preferably each, anatomical site may be a 3D scancone of 3D-distributed scanlines, similar to a scancone 300.

The term “registration” with reference to digital images means a determination of a geometrical transformation or mapping that aligns viewpoint pixels or voxels from one data cone sample of the object (in this embodiment, a heart) with viewpoint pixels or voxels from another data cone sampled at a different location from the object. That is, registration involves mathematically determining and converting the coordinates of common regions of an object from one viewpoint to coordinates of another viewpoint. After registration of at least two data cones to a common coordinate system, registered data cone images are then fused together by combining two registered data images by producing a reoriented version from a view of one of the registered data cones. That is, for example, a second data cone's view is merged into a first data cone's view by translating and rotating pixels of a second data cone's pixels that are common with pixels of a first data cone. Knowing how much to translate and rotate a second data cone's common pixels or voxels allows pixels or voxels in common between both data cones to be superimposed into approximately the same x, y, z, spatial coordinates so as to accurately portray an object being imaged. The more precise and accurate a pixel or voxel rotation and translation, the more precise and accurate is a common pixel or voxel superimposition or overlap between adjacent image cones. A precise and accurate overlap between the images assures a construction of an anatomically correct composite image mosaic substantially devoid of duplicated anatomical regions.

To obtain a precise and accurate overlap of common pixels or voxels between adjacent data cones, it is advantageous to utilize a geometrical transformation that substantially preserves most or all distances regarding line straightness, surface planarity, and angles between lines as defined by image pixels or voxels. That is, a preferred geometrical transformation that fosters obtaining an anatomically accurate mosaic image is a rigid transformation that doesn't permit the distortion or deforming of geometrical parameters or coordinates between pixels or voxels common to both image cones.

A rigid transformation first converts polar coordinate scanplanes from adjacent image cones into in x, y, z Cartesian axes. After converting scanplanes into the Cartesian system, a rigid transformation, T, is determined from scanplanes of adjacent image cones having pixels in common. A transformation T is a combination of a three-dimensional translation vector expressed in Cartesian as t=(Tₓ, T_{y}, T_{z}), and a three-dimensional rotation R matrix expressed as a function of Euler angles θₓ, θ_{y}, θ_{z}, around an x, y, and z-axes. A transformation represents a shift and rotation conversion factor that aligns and overlaps common pixels from scanplanes of adjacent image cones.

In a preferred embodiment of the present invention, the common pixels used for purposes of establishing registration of three-dimensional images are boundaries of the cardiac surface regions as determined by a segmentation algorithm described above.

FIG. 13 is a block diagram algorithm overview of a registration and correcting algorithm used in processing multiple image cone data sets. Several different protocols may be used to collect and process multiple cones of data from more than one measurement site are described in a method illustrated in FIG. 13.

FIG. 13 illustrates a block method for obtaining a composite image of a heart from multiply acquired 3D scancone images. At least two 3D scancone images are acquired at different measurement site locations within a chest region of a patient or subject under study.

An image mosaic involves obtaining at least two image cones where a transceiver 10 is placed such that at least a portion of a heart is ultrasonically viewable at one or more, or preferably each, measurement site. A first measurement site is originally defined as fixed, and a second site is defined as moving and placed at a first known inter-site distance relative to a first site. A second site images are registered and fused to first site images. After fusing a second site images to first site images, other sites may be similarly processed. For example, if a third measurement site is selected, then this site is defined as moving and placed at a second known inter-site distance relative to the fused second site now defined as fixed. Third site images are registered and fused to second site images. Similarly, after fusing third site images to second site images, a fourth measurement site, if needed, is defined as moving and placed at a third known inter-site distance relative to a fused third site now defined as fixed. Fourth site images are registered and fused to third site images.

As described above, four measurement sites may be along a line or in an array. The array may include rectangles, squares, diamond patterns, or other shapes. Preferably, a patient is positioned and stabilized and a 3D scancone images are obtained between the subjects breathing, so that there is not a significant displacement of the art while a scancone image is obtained.

An interval or distance between one or more, or preferably each, measurement site is approximately equal, or may be unequal. An interval distance between measurement sites may be varied as long as there are mutually viewable regions of portions of a heart between adjacent measurement sites. A geometrical relationship between one or more, or preferably each, image cone is ascertained so that overlapping regions can be identified between any two image cones to permit a combining of adjacent neighboring cones so that a single 3D mosaic composite image is obtained.

Translational and rotational adjustments of one or more, or preferably each, moving cone to conform with voxels common to a stationary image cone is guided by an inputted initial transform that has expected translational and rotational values. A distance separating a transceiver 10 between image cone acquisitions predicts the expected translational and rotational values. For example, expected translational and rotational values are proportionally defined and estimated in Cartesian and Euler angle terms and associated with voxel values of one or more, or preferably each, scancone image.

A block diagram algorithm overview of FIG. 13 includes registration and correcting algorithms used in processing multiple image cone data sets. An algorithm overview 1000 shows how an entire cardiac ejection fraction measurement process occurs from a plurality of acquired image cones. First, one or more, or preferably each, input cone 1004 is segmented 1008 to detect all blood fluid regions. Next, these segmented regions are used to align (register) different cones into one common coordinate system using a registration 1012 algorithm. A registration algorithm 1012 may be rigid for scancones obtained from a non-moving subject, or may be non-rigid, for scancones obtained while a patient was moving (for example, a patient was breathing during a scancone image acquisitions). Next, registered datasets from one or more, or preferably each, image cone are fused with each other using a Fuse Data 1016 algorithm to produce a composite 3D mosaic image. Thereafter, a left ventricular volumes are determined from a composite image at an end-systole and end-diastole time points, permitting a cardiac ejection fraction to be calculated from the calculate volume block 1020 from a fused or composite 3D mosaic image.

In alternate embodiments, the above steps and/or subsets may be omitted, or preceded by other steps

Volume and Ejection Fraction Calculation

After a left ventricular boundaries have been determined, we need to calculate the volume of a left ventricle.

If a segmented region is available in Cartesian coordinates in an image format, calculating the volume is straightforward and simply involves adding a number of voxels contained inside a segmented region multiplied by a volume of each voxel.

If a segmented region is available as set of polygons on set of Cartesian coordinate images, then we first need to interpolate between polygons and create a triangulated surface. A volume contained inside the triangulated surface can be then calculated using standard computer-graphics algorithms.

If a segmented region is available in a form of polygons or regions on polar coordinate images, then we can apply formulas as described in our Bladder Volume Patent to calculate the volume.

Once an end-diastolic volume (EDV) and end-systolic volumes (ESV) are calculated, an ejection fraction (EF) can be calculated as:

EF=100*(EDV−ESV)/EDV

In alternate embodiments, the above steps and/or subsets may be omitted, or preceded by other steps.

While the preferred embodiment of the invention has been illustrated and described, as noted above, many changes can be made without departing from the spirit and scope of the invention. For example, other uses of the invention include determining the areas and volumes of the prostate, heart, bladder, and other organs and body regions of clinical interest. Accordingly, the scope of the invention is not limited by the disclosure of the preferred embodiment.

In general, systems and/or methods of image processing are described for automatically segmenting, i.e. automatically detecting the boundaries of shapes within a region of interest (ROI) of a single or series of images undergoing dynamic change. Particular and alternate embodiments provide for the subsequent measurement of areas and/or volumes of the automatically segmentated shapes within the image ROI of a singular image multiple images of an image series undergoing dynamic change.

Methods include creating an image database having manually segmented shapes within the ROI of the images stored in the database, training computer readable image processing algorithms to duplicate or substantially reproduce the appearance of the manually segmented shapes, acquiring a non-database image, and segmenting shapes within the ROI of the non-database image by using the database-trained image processing algorithms.

In particular, as applied to sonographic systems, ultrasound systems and/or methods employing the acquisition of 3D transthoracic echocardiograms (TTE) are described to non-invasively measure heart chamber volumes and/or wall thicknesses between heart chambers during and/or between systole and/or diastole from 3D data sets acquired at systole and/or diastole. The measurements are obtained by using computer readable media employing image processing algorithms applied to the 3D data sets.

Moreover, these ultrasound systems and/or methods are further described to non-invasively measure heart chamber volumes, for example the left and/or right ventricle, and/or wall thicknesses and/or masses between heart chambers during and/or between systole and/or diastole from 3D data sets acquired at systole and/or diastole through the use of computer readable media having microprocessor executable image processing algorithms applied to the 3D data sets. The image processing algorithm utilizes trainable segmentation sub-algorithms. The changes in cardiac or heart chamber volumes may be expressed as a quotient of the difference between a given cardiac chamber volume occurring at systole and/or diastole and/or the volume of the given cardiac chamber at diastole. When the given cardiac chamber is the left ventricle, the changes in the left ventricle volumes may be expressed as an ejection fraction defined to be the quotient of the difference between the left ventricle volume occurring at systole and/or diastole and/or the volume of the left ventricle chamber at diastole.

The systems for cardiac imaging includes an ultrasound transceiver configured to sense the mitral valve of a heart by Doppler ultrasound, an electrocardiograph connected with a patient and synchronized with the transceiver to acquire ultrasound-based 3D data sets during systole and/or diastole at a transceiver location determined by Doppler ultrasound affected by the mitral valve, and a computer readable medium configurable to process ultrasound imaging information from the 3D data sets communicated from the transceiver and being synchronized with transceiver so that electrocardiograph connected with a patient that is configurable to determine an optimal location to acquire ultrasound echo 3D data sets of the heart during systole and/or diastole; utilize ultrasound transducers equipped with a microphone to computer readable mediums in signal communication with an electrocardiograph.

The image processing algorithms delineate the outer and/or inner walls of the heart chambers within the heart and/or determine the actual surface area, S, of a given chamber using a modification of the level set algorithms, as described below, and utilized from the VTK Library maintained by Kitware, Inc. (Clifton Park, N.Y., USA), incorporated by reference herein. The selected heart chamber, the thickness t of wall between the selected heart chamber and adjacent chamber, is then calculated as the distance between the outer and the inner surfaces of selected and adjacent chambers. Finally, as shown in equation E1, the inter-chamber wall mass (ICWM) is estimated as the product of the surface area, the interchamber wall thickness (ICWT) and cardiac muscle specific gravity, ρ:

ICWM=S×ICWTρ E1

One benefit of the embodiments of the present invention is that it produces more accurate and consistent estimates of selected heart chamber volumes and/or inter-chamber wall masses. The reasons for higher accuracy and consistency include:
1. The use of three-dimensional data instead of two-dimensional data to calculate the surface area and/or thickness. In another embodiment, the outer anterior wall of the heart chamber is delineated to enable the calculation of the inter-chamber wall thickness (ICWT);
2. The use of the trainable segmentation sub-algorithms in obtaining measured surface area instead of using surface area based upon a fixed model; and
3. The automatic and consistent measurement of the ICWT.

Additional benefits conferred by the embodiments also include its non-invasiveness and its ease of use in that ICWT is measured over a range of chamber volumes, thereby eliminating the need to invasively probe a patient.

FIGS. 1A-D depicts a partial schematic and partial isometric view of a transceiver, a scan cone array of scan planes, and a scan plane of the array.

FIG. 1A depicts a transceiver 10A having an ultrasound transducer housing 18 and a transceiver dome 20 from which ultrasound energy emanates to probe a patient or subject upon pressing the button 14. Doppler or image information from ultrasound echoes returning from the probed region is presented on the display 16. The information may be alphanumeric, pictorial, and describe positional locations of a targeted organ, such as the heart, or other chamber-containing ROI. A speaker 15 conveys audible sound indicating the flow of blood between and/or from heart chambers. Characteristic sounds indicating blow flow through and/or from the mitral valve are used to reposition the transceiver 10A for the centered acquisition of image 3D data sets obtained during systole and/or diastole.

FIG. 1B is a graphical representation of a plurality of scan planes 42 that contain the probing ultrasound. The plurality of scan planes 42 defines a scan cone 40 in the form of a three-dimensional (3D) array having a substantially conical shape that projects outwardly from the dome 20 of the transceivers 10A.

The plurality of scan planes 42 are oriented about an axis 11 extending through the transceivers 10A. One or more, or alternately each of the scan planes 42 are positioned about the axis 11, which may be positioned at a predetermined angular position θ. The scan planes 42 are mutually spaced apart by angles θ₁ and θ₂ whose angular value may vary. That is, although the angles θ₁ and θ₂ to θₙ are depicted as approximately equal, the θ angles may have different values. Other scan cone configurations are possible. For example, a wedge-shaped scan cone, or other similar shapes may be generated by the transceiver 10A.

FIG. 1C is a graphical representation of a scan plane 42. The scan plane 42 includes the peripheral scan lines 44 and 46, and an internal scan line 48 having a length r that extends outwardly from the transceivers 10A and between the scan lines 44 and 46. Thus, a selected point along the peripheral scan lines 44 and 46 and the internal scan line 48 may be defined with reference to the distance r and angular coordinate values φ and θ. The length r preferably extends to approximately 18 to 20 centimeters (cm), although other lengths are possible. Particular embodiments include approximately seventy-seven scan lines 48 that extend outwardly from the dome 20, although any number of scan lines may be used.

FIG. 1D a graphical representation of a plurality of scan lines 48 emanating from the ultrasound transceiver forming a single scan plane 42 extending through a cross-section of portions of an internal bodily organ. The scan plane 42 is fan-shaped, bounded by peripheral scan lines 44 and 46, and has a semi-circular dome cutout 41. The number and/or location of the internal scan lines emanating from the transceivers 10A within a given scan plane 42 may be distributed at different positional coordinates about the axis line 11 to sufficiently visualize structures or images within the scan plane 42. As shown, four portions of an off-centered region-of-interest (ROI) are exhibited as irregular regions 49 of the internal organ. Three portions are viewable within the scan plane 42 in totality, and one is truncated by the peripheral scan line 44.

As described above, the angular movement of the transducer may be mechanically effected and/or it may be electronically or otherwise generated. In either case, the number of lines 48 and/or the length of the lines may vary, so that the tilt angle φ (FIG. 1C) sweeps through angles approximately between −60° and +60° for a total arc of approximately 120°. In one particular embodiment, the transceiver 10A is configured to generate approximately about seventy-seven scan lines between the first limiting scan line 44 and a second limiting scan line 46. In another particular embodiment, each of the scan lines has a length of approximately about 18 to 20 centimeters (cm). The angular separation between adjacent scan lines 48 (FIG. 1B) may be uniform or non-uniform. For example, and in another particular embodiment, the angular separation φ₁ and φ₂ to φₙ (as shown in FIG. 1B) may be about 1.5°. Alternately, and in another particular embodiment, the angular separation φ₁, φ₂, φₙ may be a sequence wherein adjacent angles are ordered to include angles of 1.5°, 6.8°, 15.5°, 7.2°, and so on, where a 1.5° separation is between a first scan line and a second scan line, a 6.8° separation is between the second scan line and a third scan line, a 15.5° separation is between the third scan line and a fourth scan line, a 7.2° separation is between the fourth scan line and a fifth scan line, and so on. The angular separation between adjacent scan lines may also be a combination of uniform and non-uniform angular spacings, for example, a sequence of angles may be ordered to include 1.5°, 1.5°, 1.5°, 7.2°, 14.3°, 20.2°, 8.0°, 8.0°, 8.0°, 4.3°, 7.8°, and so on.

FIG. 2 depicts a partial schematic and partial isometric and side view of a transceiver 10B, and a scan cone array 30 comprised of 3D-distributed scan lines. Each of the scan lines have a length r that projects outwardly from the transceiver 10B. As illustrated the transceiver 10B emits 3D-distributed scan lines within the scan cone 30 that are one-dimensional ultrasound A-lines. Taken as an aggregate, these 3D-distributed A-lines define the conical shape of the scan cone 30. The ultrasound scan cone 30 extends outwardly from the dome 20 of the transceiver 10B and centered about the axis line 11 (FIG. 1B). The 3D-distributed scan lines of the scan cone 30 include a plurality of internal and peripheral scan lines that are distributed within a volume defined by a perimeter of the scan cone 30. Accordingly, the peripheral scan lines 31A-31F define an outer surface of the scan cone 30, while the internal scan lines 34A-34C are distributed between the respective peripheral scan lines 31A-31F. Scan line 34B is generally collinear with the axis 11, and the scan cone 30 is generally and coaxially centered on the axis line 11.

The locations of the internal and/or peripheral scan lines may be further defined by an angular spacing from the center scan line 34B and between internal and/or peripheral scan lines. The angular spacing between scan line 34B and peripheral or internal scan lines are designated by angle Φ and angular spacings between internal or peripheral scan lines are designated by angle Ø. The angles Φ₁, Φ₂, and Φ₃ respectively define the angular spacings from scan line 34B to scan lines 34A, 34C, and 31D. Similarly, angles Ø₁, Ø₂, and Ø₃ respectively define the angular spacing between scan line 31B and 31C, 31C and 34A, and 31D and 31E.

With continued reference to FIG. 2, the plurality of peripheral scan lines 31A-E and the plurality of internal scan lines 34A-D are three dimensionally distributed A-lines (scan lines) that are not necessarily confined within a scan plane, but instead may sweep throughout the internal regions and/or along the periphery of the scan cone 30. Thus, a given point within the scan cone 30 may be identified by the coordinates r, Φ, and Ø whose values generally vary. The number and/or location of the internal scan lines 34A-D emanating from the transceiver 10B may thus be distributed within the scan cone 30 at different positional coordinates to sufficiently visualize structures or images within a region of interest (ROI) in a patient. The angular movement of the ultrasound transducer within the transceiver 10B may be mechanically effected, and/or it may be electronically generated. In any case, the number of lines and/or the length of the lines may be uniform or otherwise vary, so that angle Φ may sweep through angles approximately between −60° between scan line 34B and 31A, and +60° between scan line 34B and 31B. Thus, the angle Φ may include a total arc of approximately 120°. In one embodiment, the transceiver 10B is configured to generate a plurality of 3D-distributed scan lines within the scan cone 30 having a length r of approximately 18 to 20 centimeters (cm). Repositioning of the transceiver 10B to acquire centered cardiac images derived from 3D data sets obtained at systole and/or diastole may also be affected by the audible sound of mitral valve activity caused by Doppler shifting of blood flowing through the mitral valve that emanates from the speaker 15.

FIG. 3 depicts a transceiver 10C acquiring a translation array 70 of scanplanes 42. The translation array 70 is acquired by successive, linear freehand movements in the direction of the double headed arrow. Sound emanating from the speaker 15 helps determine the optimal translation position arising from mitral valve blood flow Doppler shifting for acquisition of 3D image data sets during systole and/or diastole.

FIG. 4 depicts a transceiver 10D acquiring a fan array 60 of scanplanes 42. The fan array 60 is acquired by successive, incremental pivoting movement of the ultrasound transducer along the direction of the curved arrow. Sound emanating from the speaker 15 helps determine the optimal translation position arising from mitral valve blood flow Doppler shifting for acquisition of 3D image data sets during systole and/or diastole.

FIG. 6 depicts the transceivers 10A-D removably positioned in a communications cradle to communicate imaging data by wire connections uploaded to the computer or other microprocessor device (not shown). The data is uploaded securely to the computer or to a server via the computer where it is processed by a bladder weight estimation algorithm that will be described in greater detail below. The transceiver 10B may be similarly housed in the cradle 50A. In this wireless embodiment, the cradle 50A has circuitry that receives and converts the informational content of the scan cone 40 or scan cone 30 to a wireless signal 50A-2.

FIG. 6 depicts the transceivers 10A-D removably positioned in a communications cradle 50B where the data is uploaded by an electrical connection 50B-2 to the computer or other microprocessor device (not shown). The data is uploaded securely to the computer or to a server via the computer where it is processed by the bladder weight estimation algorithm. In this embodiment, the cradle 50B has circuitry that receives and converts the informational content of the scan cones 30/40, translation array 70, scanplane fan 60, scanplane to a non-wireless signal that is conveyed in conduit 50B-2 capable of transmitting electrical, light, or sound-based signals. A particular electrical embodiment of conduit 50B-2 may include a universal serial bus (USB) in signal communication with a microprocessor-based device.

FIG. 7A depicts an image showing the chest area of a patient 68 being scanned by a transceivers 10A-D and the data being wirelessly uploaded to a personal computer during initial targeting of a region of interest (ROI) of the heart (dashed lines) during an initial targeting or aiming phase. The heart ROI is targeted underneath the sternum between the thoracic rib cages at a first freehand position. Confirmation of target positioning is determined by the characteristic Doppler sounds emanating from the speaker 15.

FIG. 7B depicts an image showing the chest area of the patient 68 being scanned by a transceiver 10A-D at a second freehand position where the transceiver 10A-E is aimed toward the cardiac ROI between ribs of the left side of the thoracic cavity. Similarly, confirmation of target positioning is determined by the characteristic Doppler sounds emanating from the speaker 15.

FIG. 8 depicts the centering of the heart for later acquisition of 3D image sets based upon the placement of the mitral valve near the image center as determined by the characteristic Doppler sounds from the speaker 15 of transceivers 10A-D. A white broadside scan line on the pre-scan-converted image is visible. Along this line, the narrow band signals are transmitted and the Doppler signals are acquired.

When the ultrasound scanning device is in an aiming mode, the transducer is fixed at the broadside scan line position. The ultrasound scanning device repeats transmitting and receiving sound waves alternatively with the pulse repetition frequency, prf. The transmitting wave is narrow band signal which has large number of pulses. The receiving depth is gated between 8 cm and 15 cm to avoid the ultrasound scanning device's wall detecting of the motion artifacts from hands or organ (heartbeat).

FIG. 9 is a schematic depiction of the Doppler operation of the transceivers 10A-D described in terms of independent, range-gated, and parallel. Waves are transmitting to tissue and reflected waves are returning from tissue. The frequency of the mitral valve opening is the same as the heart bit which is 1 Hz (normally 70 times per minute). The speed of open/close motion which will relate to the Doppler frequency is approximately 10 cm/s (maximum of 50 cm/s). The interval between acquired RFUS lines represents the prf. For the parallel or pulse wave (PW) case, the relationship between the maximum mitral valve velocity, Vₘₐₓ, and prf not to have aliasing is Vₘₐₓ (λ/2)·prf. Therefore, in order to detect the maximum velocity 50 cm/s using 3.7 MHz transmit frequency while avoiding aliasing, at least 2.5 KHz prf may be used.

The CW (Continuous Wave-independent) Doppler as shown in FIG. 9 can estimate the velocities independently, i.e., each scanline has its Doppler frequency shift information. CW does not include information about the depth where the motion occurs. The range gated CW Doppler can limit the range to some extent but still should keep the number of pulses to be narrow band signal to separate the Doppler frequency from the fundamental frequency. In order to get the detailed depth with reasonable axial resolution, PW Doppler technique is used. The consecutive pulse-echo scanlines are compared parallel direction to get the velocity information.

In aiming, some range is desirable but detailed depth information is not required. Furthermore the transducer is used for imaging and the Doppler aiming, therefore, the range gated CW Doppler technique is appropriate.

The relationship between the Doppler frequency, f_{d}, and the object velocity, v₀, is according to equation E2:

$${f}_{d} = {f}_{0} \cdot \frac{{v}_{0}}{c + {v}_{0}} \approx {f}_{0} \cdot \frac{{v}_{0}}{c} \qquad E 2$$

where, f₀ is the transmit frequency and c is the speed of sound.

An average maximum velocity of the mitral valve is about 10 cm/s. If the transmit frequency, f₀, is 3.7 MHz and the speed of sound is 1540 m/s, the Doppler frequency, f_{d}, created by the mitral valve is about 240 Hz.

FIG. 10 is a system schematic of the Doppler-speaker circuit of the transceivers 10A-D. The sinusoid wave, cos(2πf₀t), is transmitted to tissue using a transducer. After certain range-gated time, the sinusoid wave with Doppler frequency component, f_{d}, is received by the transducer. The received signal can be defined as cos(2π(f₀+f_{d})t), so that by multiplying the transmit signal and received signal, m(t) is expressed according to equation E3 as:

m(t)=cos(2π(f₀+f_{d})·cos(2πf₀t) E3

Using the trigonometric Identity,

$$cos x \cdot cos y = \frac{1}{2} [ cos ( x - y ) + cos ( x + y ) ] ,$$

y)], m(t) can be rewritten as equation E4:

m(t)=cos(2π(f₀+2f_{d})t)+cos(2πf_{d}t) E4

The frequency components of m(t) are (f₀+2f_{d}) and f_{d}, which are a high frequency component and a low frequency component. Therefore using low pass filter whose cutoff frequency is higher than the Doppler frequency, f_{d}, but lower than the fundamental frequency, f₀, only the Doppler frequency, f_{d}, is remained, according to E5:

LPF{m(t)}=cos(2πf_{d}t) E5

The ultrasound scanning device's loud speaker produces the Doppler sound, when it is in the aiming mode. When the Doppler sound of the mitral valve is audible, the 3D acquisition may be performed.

FIG. 11 presents three graphs describing the operation of image acquisition using radio frequency ultrasound (RFUS) and timing to acquire RFUS images at cardiac systole and/or diastole to help determine the cardiac ejection fractions of the left and/or right ventricles. An M-mode US display in the upper left graph is superimposed by the RFUS acquisition range and is presented in the upper right graph as a frequency response of the RFUS lines. The RFUs lines are multiplied by the input sinusoid and the result includes a RFUS discontinuity artifact. The green line in the bottom graph is the filtered signal using an average filter. The time domain representations are of RFUS, multiplied RFUS, and filtered Doppler signal.

FIG. 12 illustrates system 60A for beginning of acquiring 3D data sets acquired during 3D transthoracic echocardiogram procedures. The transceiver 10A-D is placed beneath the sternum at a first freehand position with the scan head 20 aimed slightly towards the apical region of the heart. The heart is shown beneath the sternum and rib cage as in a dashed outline. The three-dimensional ultrasound data is collected during systole and/or diastole at an image-centering position indicated by audible sounds characteristic of Doppler shifts associated with the mitral valve. In concert with the electrocardiograph as explained below, 3D image data sets are acquired at systole and/or diastole upon pressing the scan button 14 on the transceivers 10A-D. After the 3D data set scans are complete, the display 16 on the devices 10A-D displays aiming information in the form of arrows, or alternatively, by sound maxima arising from Doppler shifts. A flashing arrow indicates to the user to point the device in the arrow's direction and rescan at systole or diastole as needed. The scan is repeated until the device displays only a solid arrow or no arrow. The display 16 on the device may also display the calculated ventricular or atrial chamber volumes at systole and/or diastole. The aforementioned aiming process is more fully described in U.S. Pat. No. 6,884,217 to McMorrow et al., which is incorporated by reference as if fully disclosed herein. Once the systole and/or diastole image scanning is complete, the device may be placed on a communication cradle that is attached to a personal computer. Other methods and systems described below incorporate by reference U.S. Pat. Nos. 4,926,871; 5,235,985; 6,569,097; 6,110,111; 6,676,605; 7,004,904; and 7,041,059 as if fully disclosed herein.

The transceiver 10A-D has circuitry that converts the informational content of the scan cones 40/30, translational array 70, or fan array 60 to wireless signal 25C-1 that may be in the form of visible light, invisible light (such as infrared light) or sound-based signals. As depicted, the data is wirelessly uploaded to the personal computer 52 during initial targeting of the heart or other cavity-containing ROI. In a particular embodiment of the transceiver 10A-D, a focused 3.7 MHz single element transducer is used that is steered mechanically to acquire a 120-degree scan cone 42. On a display screen 54 coupled to the computer 52, a scan cone image 40A displays an off-centered view of the heart 56A that is truncated.

Expanding on the protocol described above, and still referring to FIG. 12 the system 60A also includes a personal computing device 52 that is configured to wirelessly exchange information with the transceiver 10C, although other means of information exchange may be employed when the transceiver 10C is used. In operation, the transceiver 10C is applied to a side abdominal region of a patient 68. The transceiver 10B is placed off-center from of the thoracic cavity of the patient 68 to obtain, for example a sub-sternum image of the heart. The transceiver 10B may contact the patient 68 through an ultrasound conveying gel pad that includes an acoustic coupling gel that is placed on the patient 68 sub sternum area. Alternatively, an acoustic coupling gel may be applied to the skin of the patient 68. The pad 67 advantageously minimizes ultrasound attenuation between the patient 68 and the transceiver 10B by maximizing sound conduction from the transceiver 10B into the patient 68.

Wireless signals 25C-1 include echo information that is conveyed to and processed by the image processing algorithm in the personal computer device 52. A scan cone 40 (FIG. 1B) displays an internal organ as partial image 56A on a computer display 54. The image 56A is significantly truncated and off-centered relative to a middle portion of the scan cone 40A due to the positioning of the transceiver 10B.

As shown in FIG. 12, the sub-sternum acquired images are initially obtained during a targeting phase of the imaging. During the initial targeting, a first freehand position may reveal an organ, for example the heart or other ROI 56A that is substantially off-center. The transceivers 10A-D are operated in a two-dimensional continuous acquisition mode. In the two-dimensional continuous mode, data is continuously acquired and presented as a scan plane image as previously shown and described. The data thus acquired may be viewed on a display device, such as the display 54, coupled to the transceivers 10A-D while an operator physically repositions the transceivers 10A-D across the chest region of the patient. When it is desired to acquire data, the operator may acquire data by depressing the trigger 14 of the transceivers 10A-D to acquire real-time imaging that is presented to the operator on the transceiver display 16. If the initial location of the transceiver is significantly off-center, as in the case of the freehand first position, results in only a portion of the organ or cardiac ROI 56A being visible in the scan plane 40A.

FIG. 13 depicts images showing the patient 68 being scanned by the transceivers 10A-D and the data being wirelessly uploaded to a personal computer of a properly targeted cardiac ROI in the left thoracic area between adjacent ribs showing a centered heart or cardiac ROI 56B as properly targeted. The isometric view presents the ultrasound imaging system 60A applied to a centered cardiac region of the patient. The transceiver 10A-D may be translated or moved to a freehand second position between ribs having an apical view of the heart. Wireless signals 25C-2 having information from the transceiver 10C are communicated to the personal computer device 52. An inertial reference unit positioned within the transceiver 10A-D senses positional changes for the transceiver 10C relative to a reference coordinate system. Information from the inertial reference unit, as described in greater detail below, permits updated real-time scan cone image acquisition, so that a scan cone 40B having a complete image of the organ 56B can be obtained.

FIG. 14 depicts an alternate embodiment 70A of the cardiac imaging system using an electrocardiograph in communication with a wireless ultrasound transceiver. System 70A includes the speaker 15 equipped transceiver 10A-D in wireless signal communication with an electrocardiograph 74 and the personal computer device 52. The electrocardiograph 74 includes a display 76 is in wired communication with the patient through electrical contacts 78. Cardio activity of the patient's heart is shown as a PQRST wave on display 76 in which the timing for acquisition of 3D datasets at systole and diastole may be undertaken when the heart 56B is centered within the scan cone 40B on the display 54 of the computing device 52. Wireless signal 80 from the electrocardiograph 74 signals the transceiver 10A-D for acquisition of 3D datasets at systole and diastole which in turn is wireless transmitted to the personal computer device 52. Other information from the electrocardiograph 74 to the personal computer device 52 may be conveyed via wireless signal 82.

FIG. 15 depicts an alternate embodiment 70B of the cardiac imaging system using an electrocardiograph in communication with a wired connected ultrasound transceiver. System 70B includes wired cable 84 connecting the electrocardiograph 74 and speaker-equipped transceivers 10A-D and cable 86 connecting the transceivers 10A-D to the computing device 52. Similar in operation to wireless system 70A, the electrocardiograph 74 signals the transceiver 10A-D for acquisition of 3D datasets at systole and diastole via cable 84 and information of the 3D datasets are conveyed to the computer device 52 via cable 86. Other information from the electrocardiograph 74 to the personal computer device 52 may be conveyed via wireless signal 82. Alternatively, the electrocardiograph 74 may convey signals directly to the computing device 52 by wired cables.

Alternate embodiments of systems 70A and 70B allow for different signal sequence communication between the transceivers 10A-D, 10E, electrocardiograph 74 and computing device 52. That is, different signal sequences may be used in executing the timing of diastole and systole image acquisition. For example, the electrocardiograph 74 may signal the computing device 52 to trigger the transceivers 10A-D and 10E to initiate image acquisition at systole and diastole.

FIG. 16 schematically depicts an alternate embodiment of the cardiac imaging system during Doppler targeting with microphone equipped transceivers 10A-D. Mitral valve mitigation of Doppler shifting is audibly recognizable as the user moves the transceiver A-D to different chest locations to find a chest region to acquire systole and/or diastole centered 3D data sets. Audible wave set 90 is heard by the sonographer emanating from transceiver's 10A-D speaker 15. The cardio activity PQRST is presented on display 76 of the electrocardiograph 74.

FIG. 17 schematically depicts an alternate embodiment of the cardiac imaging system during Doppler targeting of a speaker-less transceiver 10E with a speaker-equipped electrocardiograph. Similar in operation to the alternate embodiment of FIG. 16, in this schematic the alternate embodiment includes the speaker or speakers 74A located on the electrocardiograph 74. Upon a user moving the transceiver 10E to different chest locations, the mitral mitigating Doppler shift is heard from electrocardiograph speakers 74A released as audio wave sets 94 to indicate optimal mitral valve centering at a given patient chest location for subsequent acquisition of the systole and/or diastole centered 3D data sets.

FIG. 18 is a schematic illustration and partial isometric view of a network connected cardio imaging ultrasound system 100 in communication with ultrasound imaging systems 60A-D. The system 100 includes one or more personal computer devices 52 that are coupled to a server 56 by a communications system 55. The devices 52 are, in turn, coupled to one or more ultrasound transceivers 10A-D in systems 60A-B used with the 3D datasets downloaded to the computer 52 substantially operating simultaneously with the electrocardiographs, or transceivers 10A-E of systems 60C-D where the systole and/or diastole 3D data sets are downloaded from the cradles 50A-B sequentially and separate from the electrocardiographs. The server 56 may be operable to provide additional processing of ultrasound information, or it may be coupled to still other servers (not shown in FIG. 17) and devices, for examples transceivers 10E may be equipped with a snap on collars having speaker configured to audibly announce changes in mitral valve mitigated Doppler shifting. Once the systole and/or diastole scans are complete, the three-dimensional data may be transmitted securely to a server computer on a remote computer that is coupled to a network, such as the Internet.

Alternately, a local computer network, or an independent standalone personal computer may also be used. In any case, image processing algorithms on the computer analyze pixels within a 2D portion of a 3D image or the voxels of the 3D image. The image processing algorithms then define which pixels or voxels occupy or otherwise constitute an inner or outer wall layer of a given wall chamber. Thereafter, wall areas of the inner and outer chamber layers, and thickness between them, is determined. Inter-chamber wall weight is determined as a product of wall layer area, thickness between the wall layers, and density of the wall.

FIG. 19 is a schematic illustration and partial isometric view of an Internet connected cardio imaging ultrasound system 110 in communication with ultrasound imaging systems 60A-D. The Internet system 110 is coupled or otherwise in communication with the systems 60A-60D. The system 110 may also be in communication with the transceiver a snap on microphone collar described above.

FIG. 20 is an algorithm flowchart 200 for the method to measure and determine heart chamber volumes, changes in heart chamber volumes, ICWT and ICWM and begins with two entry points depending if a new training database of sonographer or manually segmented images is being created and/or expanded, or whether a pre-existing and developed sonographer database is being used. In the case wherein the sonographer database is being created and/or expanded, at entry point Start-1, an image database of manually segmented ROIs is created by an expert sonographer at process block 204. Alternatively, entry point Start-1 may begin at process block 224, wherein an image database of manually segmented ROIs is created that is enhanced by a Radon Transform by an expert sonographer. Thereafter, at process block 260, image-processing algorithms are trained to substantially reproduce the appearance of the manually segmented ROIs contained in the database by the use of created statistical shape models as further described below. Once the level set algorithms are trained on the manually segmented image collections, algorithm 200 continues at process block 280 where new or non-database images are acquired from 3D transthoracic echocardiographic procedures obtained from any of the aforementioned systems. The non-database images are composed of 3D data sets acquired during systole and diastole as further described below. If the combined database from process blocks 204 and 224 is already created and developed, an alternate entry point is depicted by entering algorithm flowchart 200 via Start-2 into process block 260 for acquisition of non-database images at systole and diastole. After acquisition of non-database images, algorithm 200 continues at process block 300 where structures within the ROI of the non-database 3D data sets are segmented using the trained image processing algorithms from process block 260. Finally, the algorithm 200 is completed at process block 310 where at least one of ICWT, ICWM, and the ejection fraction of at least one heart chamber is determined from information of the segmented structures of the non-database image.

FIG. 21 is an expansion of sonographer-executed sub-algorithm 204 of flowchart in FIG. 20 that utilizes a 2-step enhancement process. 3D data sets are entered at input data process block 206 which then undergoes a 2-step image enhancement procedure at process block 208. The 2-step image enhancement includes performing a heat filter to reduce noise followed by a shock filter to sharpen edges of structures within the 3D data sets. The heat and shock filters are partial differential equations (PDE) defined respectively in Equations E6 and E7 below:

$$\begin{matrix}\frac{\partial u}{\partial t} = \frac{{\partial }^{2} u}{\partial {x}^{2}} + \frac{{\partial }^{2} u}{\partial {y}^{2}} ( Heat Filter ) & E 6 \\ \frac{\partial u}{\partial t} = - F ( 𝓁 ( u ) ) ‖ ∇ u ‖ ( Shock Filter ) & E 7\end{matrix}$$

Here u in the heat filter represents the image being processed. The image u is 2D, and is comprised of an array of pixels arranged in rows along the x-axis, and an array of pixels arranged in columns along the y-axis. The pixel intensity of each pixel in the image u has an initial input image pixel intensity (I) defined as u₀=I. The value of I depends on the application, and commonly occurs within ranges consistent with the application. For example, I can be as low as 0 to 1, or occupy middle ranges between 0 to 127 or 0 to 512. Similarly, I may have values occupying higher ranges of 0 to 1024 and 0 to 4096, or greater. For the shock filter u represents the image being processed whose initial value is the input image pixel intensity (I): u₀=I where the l(u) term is the Laplacian of the image u, F is a function of the Laplacian, and ∥∇u∥ is the 2D gradient magnitude of image intensity defined by equation E8:

$$‖ ∇ u ‖ = \sqrt{{u}_{x}^{2} + {u}_{y}^{2}} \qquad E 8$$

Where u²ₓ=the square of the partial derivative of the pixel intensity (u) along the x-axis, u²_{y}=the square of the partial derivative of the pixel intensity (u) along the y-axis, the Laplacian l(u) of the image, u, is expressed in equation E9:

l(u)=uₓₓuₓ²+2u_{xy}uₓu_{y}+u_{yy}u_{y}² E9

Equation E9 relates to equation E6 as follows:

uₓ is the first partial derivative ∂u/∂x of u along the x-axis,

uₓu_{y} is the first partial derivative ∂u/∂y of u along the y-axis,

uₓuₓ² is the square of the first partial derivative ∂u/∂x of u along the x-axis,

uₓu_{y}² is the square of the first partial derivative ∂u/∂y of u along the y-axis,

uₓuₓₓ is the second partial derivative ∂²u/∂x² of u along the x-axis,

uₓu_{yy} is the second partial derivative ∂²u/∂y² of u along the y-axis,

u_{xy} is cross multiple first partial derivative ∂u/∂xdy of u along the x and y axes, and

u_{xy} the sign of the function F modifies the Laplacian by the image gradient values selected to avoid placing spurious edges at points with small gradient values:

$$\begin{matrix}F ( 𝓁 ( u ) ) = 1 , if 𝓁 ( u ) > 0 and ‖ ∇ u ‖ > t \\ = - 1 , if 𝓁 ( u ) Z < 0 and ‖ ∇ u ‖ > t \\ = 0 , otherwise \end{matrix}$$

where t is a threshold on the pixel gradient value ∥∇u∥.

The combination of heat filtering and shock filtering produces an enhanced image ready to undergo the intensity-based and edge-based segmentation algorithms as discussed below. The enhanced 3D data sets are then subjected to a parallel process of intensity-based segmentation at process block 210 and edge-based segmentation at process block 212. The intensity-based segmentation step uses a “k-means” intensity clustering technique where the enhanced image is subjected to a categorizing “k-means” clustering algorithm. The “k-means” algorithm categorizes pixel intensities into white, gray, and black pixel groups. Given the number of desired clusters or groups of intensities (k), the k-means algorithm is an iterative algorithm comprising four steps: Initially determine or categorize cluster boundaries by defining a minimum and a maximum pixel intensity value for every white, gray, or black pixels into groups or k-clusters that are equally spaced in the entire intensity range. Assign each pixel to one of the white, gray or black k-clusters based on the currently set cluster boundaries. Calculate a mean intensity for each pixel intensity k-cluster or group based on the current assignment of pixels into the different k-clusters. The calculated mean intensity is defined as a cluster center. Thereafter, new cluster boundaries are determined as mid points between cluster centers. The fourth and final step of intensity-based segmentation determines if the cluster boundaries significantly change locations from their previous values. Should the cluster boundaries change significantly from their previous values, iterate back to step 2, until the cluster centers do not change significantly between iterations. Visually, the clustering process is manifest by the segmented image and repeated iterations continue until the segmented image does not change between the iterations.

The pixels in the cluster having the lowest intensity value—the darkest cluster—are defined as pixels associated with internal regions of cardiac chambers, for example the left or right ventricles of the left and/or right atriums. For the 2D algorithm, each image is clustered independently of the neighboring images. For the 3D algorithm, the entire volume is clustered together. To make this step faster, pixels are sampled at 2 or any multiple sampling rate factors before determining the cluster boundaries. The cluster boundaries determined from the down-sampled data are then applied to the entire data.

The edge-based segmentation process block 212 uses a sequence of four sub-algorithms. The sequence includes a spatial gradients algorithm, a hysteresis threshold algorithm, a Region-of-Interest (ROI) algorithm, and a matching edges filter algorithm. The spatial gradient algorithm computes the x-directional and y-directional spatial gradients of the enhanced image. The hysteresis threshold algorithm detects salient edges. Once the edges are detected, the regions defined by the edges are selected by a user employing the ROI algorithm to select regions-of-interest deemed relevant for analysis.

Since the enhanced image has very sharp transitions, the edge points can be easily determined by taking x- and y-derivatives using backward differences along x- and y-directions. The pixel gradient magnitude ∥∇I∥ is then computed from the x- and y-derivative image in equation E10 as:

∥∇I∥=\sqrt{Iₓ²+I_{y}²} E10

Where I²ₓ=the square of x-derivative of intensity and I²_{y}=the square of y-derivative of intensity along the y-axis.

Significant edge points are then determined by thresholding the gradient magnitudes using a hysteresis thresholding operation. Other thresholding methods could also be used. In hysteresis thresholding 530, two threshold values, a lower threshold and a higher threshold, are used. First, the image is thresholded at the lower threshold value and a connected component labeling is carried out on the resulting image. Next, each connected edge component is preserved which has at least one edge pixel having a gradient magnitude greater than the upper threshold. This kind of thresholding scheme is good at retaining long connected edges that have one or more high gradient points.

In the preferred embodiment, the two thresholds are automatically estimated. The upper gradient threshold is estimated at a value such that at most 97% of the image pixels are marked as non-edges. The lower threshold is set at 50% of the value of the upper threshold. These percentages could be different in different implementations. Next, edge points that lie within a desired region-of-interest are selected. This region of interest algorithm excludes points lying at the image boundaries and points lying too close to or too far from the transceivers 10A-D. Finally, the matching edge filter is applied to remove outlier edge points and fill in the area between the matching edge points.

The edge-matching algorithm is applied to establish valid boundary edges and remove spurious edges while filling the regions between boundary edges. Edge points on an image have a directional component indicating the direction of the gradient. Pixels in scanlines crossing a boundary edge location can exhibit two gradient transitions depending on the pixel intensity directionality. Each gradient transition is given a positive or negative value depending on the pixel intensity directionality. For example, if the scanline approaches an echo reflective bright wall from a darker region, then an ascending transition is established as the pixel intensity gradient increases to a maximum value, i.e., as the transition ascends from a dark region to a bright region. The ascending transition is given a positive numerical value. Similarly, as the scanline recedes from the echo reflective wall, a descending transition is established as the pixel intensity gradient decreases to or approaches a minimum value. The descending transition is given a negative numerical value.

Valid boundary edges are those that exhibit ascending and descending pixel intensity gradients, or equivalently, exhibit paired or matched positive and negative numerical values. The valid boundary edges are retained in the image. Spurious or invalid boundary edges do not exhibit paired ascending-descending pixel intensity gradients, i.e., do not exhibit paired or matched positive and negative numerical values. The spurious boundary edges are removed from the image.

For cardiac chamber volumes, most edge points for blood fluid surround a dark, closed region, with directions pointing inwards towards the center of the region. Thus, for a convex-shaped region, the direction of a gradient for any edge point, the edge point having a gradient direction approximately opposite to the current point represents the matching edge point. Those edge points exhibiting an assigned positive and negative value are kept as valid edge points on the image because the negative value is paired with its positive value counterpart. Similarly, those edge point candidates having unmatched values, i.e., those edge point candidates not having a negative-positive value pair, are deemed not to be true or valid edge points and are discarded from the image.

The matching edge point algorithm delineates edge points not lying on the boundary for removal from the desired dark regions. Thereafter, the region between any two matching edge points is filled in with non-zero pixels to establish edge-based segmentation. In a preferred embodiment of the invention, only edge points whose directions are primarily oriented co-linearly with the scanline are sought to permit the detection of matching front wall and back wall pairs of a cardiac chamber, for example the left or right ventricle.

Referring again to FIG. 21, results from the respective segmentation procedures are then combined at process block 214 and subsequently undergoes a cleanup algorithm process at process block 216. The combining process of block 214 uses a pixel-wise Boolean AND operator step to produce a segmented image by computing the pixel intersection of two images. The Boolean AND operation represents the pixels of each scan plane of the 3D data sets as binary numbers and the corresponding assignment of an assigned intersection value as a binary number 1 or 0 by the combination of any two pixels. For example, consider any two pixels, say pixel_{A} and pixel_{B}, which can have a 1 or 0 as assigned values. If pixel_{A}'s value is 1, and pixel_{B}'s value is 1, the assigned intersection value of pixel_{A} and pixel_{B} is 1. If the binary value of pixel_{A} and pixel_{B} are both 0, or if either pixel_{A} or pixel_{B} is 0, then the assigned intersection value of pixel_{A} and pixel_{B} is 0. The Boolean AND operation takes the binary any two digital images as input, and outputs a third image with the pixel values made equivalent to the intersection of the two input images.

After combining the segmentation results, the combined pixel information in the 3D data sets In a fifth process is cleaned at process block 216 to make the output image smooth and to remove extraneous structures not relevant to cardiac chambers or inter-chamber walls. Cleanup 216 includes filling gaps with pixels and removing pixel groups unlikely to be related to the ROI undergoing study, for example pixel groups unrelated to cardiac structures. Segmented and clean structures are then outputted to process block 262 of FIG. 23 below, and/or processed in block 218 for determination of ejection fraction of ventricles or atria, or to calculate other cardiac parameters (ICWT, ICWM). The calculation of ejection fractions or inter-chamber wall masses in block 218 may require the area or the volume of the segmented region-of-interest to be computed by multiplying pixels by a first resolution factor to obtain area, or voxels by a second resolution factor to obtain volume. For example, for pixels having a size of 0.8 mm by 0.8 mm, the first resolution or conversion factor for pixel area is equivalent to 0.64 mm², and the second resolution or conversion factor for voxel volume is equivalent to 0.512 mm³. Different unit lengths for pixels and voxels may be assigned, with a proportional change in pixel area and voxel volume conversion factors.

FIG. 22 is an expansion of sonographer-executed sub-algorithm 224 of flowchart in FIG. 20 that utilizes a 3-step enhancement process. radon transform enhancement. 3D data sets are entered at input data process block 226 which then undergoes a 3-step image enhancement procedure at process blocks 228 (radon transform), 230 (heat filter), and 232 (shock filter). The heat and shock filters 230 and 232 are substantially the same as the heat and shock filters of the image enhancement process block 208 of FIG. 21. The radon transform enhancement block 228 improves the contrast of the image sets by the application of horizontal and vertical filters to the pixels by applying an integral function across scan lines within the scan planes of the 3D data sets. The effect of the radon transform is to provide a reconstructed image from multi-planar scans and presents an image construct as a collection of blurred sinusoidal lines with different amplitudes and phases. After performing the radon transform, the reconstructed image is then subjected to the respective sequence of the heat filter 230 followed the shock filter 232. Thereafter, segmentation via parallel procedures are respectively undertaken with a 3-step region-based segmentation comprising blocks 234 (estimate shadow regions), 236 (automatic region threshold) and 238 (remove shadow regions) in parallel with and a 2-step edge-based segmentation comprising blocks 240 (spatial gradients) and 242 (hysteresis threshold of gradients).

The estimate shadow regions 234 looks for structures hidden in dark or shadow regions of scan planes within 3D data sets that would complicate the segmentation of heart chambers (for example, the segmentation of the left ventricle boundary) were they not known and segmentation artifacts or noise accordingly compensated before determining ejection fractions (See FIG. 53 below for example of boundary artifacts that appear by engaging the estimate shadow regions algorithm 234). The automatic region threshold 236 block, in a particular embodiment, automatically estimates two thresholds, an upper and a lower gradient threshold. The upper gradient threshold is estimated at a value such that at most 97% of the image pixels are marked as non-edges. The lower threshold is set at 50% of the value of the upper threshold. These percentages could be different in alternate embodiments. Next, edge points that lie within a desired region-of-interest are selected and those points lying at the image boundaries or too close or too far from the transceivers 10A-D are excluded. Finally, shadow regions are removed at process block 238 by removing image artifacts or interferences from non-chamber regions of the scan planes. For example, wall artifacts are removed from the left ventricle.

The spatial gradient 240 computes the x-directional and y-directional spatial gradients of the enhanced image. The hysteresis threshold 242 algorithm detects significant edge points of salient edges. The edge points are determined by thresholding the gradient magnitudes using a hysteresis thresholding operation. Other thresholding methods could also be used. In the hysteresis thresholding 242 block, two threshold values, a lower threshold and a higher threshold, are used. First, the image is thresholded at the lower threshold value and a connected component labeling is carried out on the resulting image. Next, each connected edge component is preserved which has at least one edge pixel having a gradient magnitude greater than the upper threshold. This kind of thresholding scheme is good at retaining long connected edges that have one or more high gradient points. Once the edges are detected, the regions defined by the edges are selected by employing the sonographer's expertise in selecting a given ROI deemed relevant by the sonographer for further processing and analysis.

Referring still to FIG. 22, a combine region and edges algorithm 244 is applied to parallel segmentation processes above in a manner substantially similar to the combine block 214 of FIG. 21. The combined results from process block 244 are then subjected to a morphological cleanup process 246 in which cleanup is achieved by removing pixel sets whose size is smaller than a structuring pixel element of a pixel group cluster. Thereafter, a snakes-based cleanup block 248 is applied to the morphologically cleaned data sets wherein the snakes cleanup is not limited to using a stopping edge-function based on the gradient of the image for the stopping process, but instead can detect contours both with and without gradients. For example, shapes having very smooth boundaries or discontinuous boundaries. In addition, the snake-base cleanup block 248 includes a level set formulation to allow the automatic detection of interior contours with the initial curve positionable anywhere in the image. Thereafter, at terminator block 250, the segmented image is outputted to block 262 of FIG. 23.

FIG. 23A is an expansion of sub-algorithm 260 of flowchart algorithm depicted in FIG. 20. Sub-algorithm 260 employs level set algorithms and constitutes a training phase section comprised of four process blocks. The first process block 262, acquire training shapes, is entered from either segmented image cleanup block 216 of FIG. 21 or output segmented image block 250 of FIG. 22. Once training shapes are acquired, the training phase continues with level set algorithms employed in blocks 264 (align shape by gradient descent), 266 (generate signed distance map), and 268 (extract mean shape and Eigen shapes). The training phase is then concluded and exits to process block 280 for acquiring a non-database image further described in FIG. 24 below.

FIG. 23B is an expansion of sub-algorithm 300 of flowchart algorithm depicted in FIG. 20 for application to non-database images acquired in process block 280. Sub-algorithm 300 constitutes the segmentation phase of the trained level set algorithms and begins by entry from process 280 wherein the non-database images are first subjected to intensity gradient analysis in a minimize shape parameters by gradient descent block 302. After gradient descent block 302, an Update shape image value ( ) block 304 using level set algorithms described by equations E11-E19 below. Once the image (d) value has been updated, then at process block 306, the inside and outside curvature C-lines from the updated image value ( ) is determined. Thereafter, a decision diamond 308 presents the query “Do inside and outside C-lines converge?”—and if the answer is negative, sub-algorithm 300 returns to process block 302 for re-iteration of the segmentation phase. If the answer is affirmative, then the segmentation phase is complete and sub-algorithm 300 then exits to process block 310 of algorithm 200 for determination of at least one of ICWT, ICWM, and ejection fraction using the segmentation results of the non-database image obtained by application of the trained level set algorithms.

FIG. 24 is an expansion of sub-algorithm 280 of flowchart 280 flowchart in FIG. 20. Entering from process 276, the speaker-equipped ultrasound transceiver 10A-D is positioned over the chest wall to scan at least a portion of the heart and receive ultrasound echoes returning from the exterior and internal surfaces of the heart per process block 282. Alternatively, the non-speaker equipped transceiver 10E is positioned over the chest wall and Doppler sounds characteristic for detecting maximum mitral valve centering is heard from speakers connected with the electrocardiograph 74. At process block 284, Doppler signals are generated in proportion to the echoes, and the Doppler signals are processed to sense the presence of the mitral valve. At decision diamond 286, a query “Is heart sufficiently targeted” is presented. If affirmative for sufficient targeting because Doppler sounds emanating from the transceiver 10A-D speaker 15 (or speakers of electrocardiograph 74) is indicative of sufficient detection of the mitral valve, then sub-algorithm 280 proceeds to process block 290 wherein 3D data sets are acquired at systole and diastole. If negative for sufficient heart targeting, the at process block 288 the transceiver 10A-D or transceiver 10E is repositioned over the chest wall to a location that generates Doppler signals indicating the maximum likelihood of mitral valve detection and centering so that acquisition of 3D data sets per step 290 may proceed. After acquisition of systole and diastole 3D data sets, the 3D data sets are then processed using trained level set algorithms per process block 292. Sub-algorithm 280 is completed and exits to sub-algorithm 300.

FIG. 25 is an expansion of sub-algorithm 310 of flowchart in FIG. 20. Entering from process block 292, adjacent cardiac chamber boundaries are delineated at process block 312 using the database trained level set algorithms. Alternatively, the ICWT is measured at block 316, or may be measured after block 312. The surface areas along the heart chamber volumes are calculated at process block 314. Thereafter, the volume between the heart chambers and the volume of the heart chambers at systole and diastole are determined at process block 320 knowing the surface area from block 314 and the thickness from block 316. From block 320, the ICWM, Left Ventricle ejection fraction, and Right Ventricle Ejection fraction may be respectively calculated at process blocks 322, 324, and 328. In the case of the Left or right Atria, the respective volumes and ejection fractions may be calculated as is done for the Left and Right Ventricles.

FIG. 26 is an 8-image panel exemplary output of segmenting the left ventricle by processes of sub-algorithm 220. The 8-image panel represents an exemplary output of segmenting the left ventricle by processes of sub-algorithm 220. Panel images include (a) Original Image, (b) After radon-transform-based image enhancement, (c) After heat & shock-based image enhancement (d) Shadow region detection result (e) Intensity segmentation result (f) Edge-detection segmentation result (g) Combination of intensity and edge-based segmentation results (h) After morphological cleanup, (i) after snakes-based cleanup (j) segmented region overlaid on the original image.

FIG. 27 presents a scan plane image with ROI of the heart delineated with echoes returning from 3.5 MHz pulsed ultrasound. Here the right ventricle (RV) and left ventricle (LV) is shown as dark chambers with an echogenic or brighter appearing wall (W) interposed between the ventricles. Beneath the bottom fan portion of the scan plane 242 is a PQRST cardiac wave tracing to help determine when 3D data sets can be acquired at systole and/or diastole.

FIG. 28 is a schematic of application of snakes processing block of sub-algorithm 248 to an active contour model. Here an abrupt transition between a circularly shaped dark region from external bright regions is mitigated by an edge function curve F. The snakes processing block relies upon edge-function F to detect objects defined by a gradient −α|VI| that produces an asymptotic curve distribution e^{-α|VI|} in the plot of F vs. |VI|. Depending on the image gradient, the curve evolution becomes limited. Geometric active contours are represented implicitly as level set functions and evolve according to an Eulerian formulation. These geometric active contours are intrinsic and advantageously are independent of the parameterization of evolving contours since parameterization doesn't occur until the level set function is completed, thereby avoiding having to add or remove nodes from an initial parameterization or to adjust the spacing of the nodes as in parametric models. The intrinsic geometric properties of the contour such as the unit normal vector and the curvature can be easily computed from the level set function. This contrasts with the parametric case, where inaccuracies in the calculations of normals and curvature result from the discrete nature of the contour parameterization. Third, the propagating contour can automatically change topology in geometric models (e.g., merge or split) without requiring an elaborate mechanism to handle such changes as in parametric models. Fourth, the resulting contours do not contain self-intersections, which are computationally costly to prevent in parametric deformable models.

There are many advantages of geometric deformable models among them the Level Set Methods are increasingly used for image processing in a variety of applications. Front evolving geometric models of active contours are based on the theory of curve evolution, implemented via level set algorithms. The automatically handle changes in topology when numerically implemented using level sets. Hence, without resorting to dedicated contour tracking, unknown numbers of multiple objects can be detected simultaneously. Evolving the curve C in normal direction with speed F amounts to solve the differential equation according to equation E11:

$$\begin{matrix}\frac{\partial Φ}{\partial t} = \vert ∇ Φ \vert F , \\ Φ ( 0 , x , y ) = {Φ}_{0} ( x , y ) & E 11 \\ \frac{\partial Φ}{\partial t} = \vert ∇ Φ \vert g ( \vert ∇ {u}_{0} \vert ) ( div ( \frac{∇ Φ}{\vert ∇ Φ \vert} ) + γ ) & E 12\end{matrix}$$

A geodesic model has been proposed. This is a problem of geodesic computation in a Riemannian space, according to a metric induced by the image. Solving the minimization problem consists in finding the path of minimal new length in that metric according to equation E13:

$$J ( C ) = 2 {\int }_{0}^{1} \vert {C}^{′} ( s ) \vert \cdot g ( \vert ∇ {u}_{0} ( C ( s ) ) \vert ) d s \qquad E 13$$

where the minimizer C can be obtained when $g(|\nabla u_{—}0 (C(s))|)$ vanishes, i.e., when the curve is on the boundary of the object. The geodesic active contour model also has a level set formulation as following, according to equation E14:

$$\frac{\partial Φ}{\partial t} = \vert ∇ Φ \vert ( div ( g ( \vert ∇ {u}_{0} \vert ) \frac{∇ Φ}{\vert ∇ Φ \vert} ) + vg ( \vert ∇ {u}_{0} \vert ) ) \qquad E 14$$

The geodesic active contour model is based on the relation between active contours and the computation of geodesics or minimal distance curves. The minimal distance curve lies in a Riemannian space whose metric is defined by the image content. This geodesic approach for object segmentation allows connecting classical “snakes” based on energy minimization and geometric active contours based on the theory of curve evolution. Models of geometric active contours are used, allowing stable boundary detection when their gradients suffer from large variations.

In practice, the discrete gradients are bounded and then the stopping function is not zero on the edges, and the curve may pass through the boundary. If the image is very noisy, the isotropic smoothing Gaussian has to be strong, which can smooth the edges too. This region based active contour method is a different active contour model, without a stopping edge-function, i.e. a model which is not based on the gradient of the image for the stopping process. A kind of stopping term is based on Mumford-Shah segmentation techniques. In this way, the model can detect contours either with or without gradient, for instance objects with very smooth boundaries or even with discontinuous boundaries. In addition, the model has a level set formulation, interior contours are automatically detected, and the initial curve can be anywhere in the image. The original Mumford-Shah functional (D. Mumford and J. Shah, “Optimal approximations by piecewise smooth functions and associated variational problems”, Comm. Pure App. Math., vol. 42, pp. 577-685, 1989) is defined by equation E15:

F^{MS}(u,C)=μLength(C)+λ∫_{Ω|u}₀(x,y)−u(x,y)|²dxdy+λ∫_{Ω\C}∇u(x,y)|²dxdy E15

The smaller the Mumford-Shah F, the segmentation improves as u₀ approximates original image u, u₀ does not vary too much on each segmented region Rᵢ, and the boundary C is as short as possible. Under these conditions u₀ it becomes a new image of the original image u drawn with sharp edges. The objects are drawn smoothly without texture. These new images are perceived correctly as representing the same scene as a simplification of the scene containing most of its features.

FIG. 29 is a schematic of application of level-set processing block of sub-algorithm 250 to an active contour model depicted by a dark circle partially merged with a dark square. Here the level set approach may solve the modified Mumford-Shah functional. In order to explain the model clearly, the evolving curve C is defined in terms of Ω. as for example, the boundary of an open subset w of Ω. In what follows, inside(C) denotes the region w, and outside(C) denotes the region Ω* W̅. The method is the minimization of an energy based-segmentation. Assume that the image u₀ is formed by two regions of approximately piecewise-constant intensities, of distinct values u₀ⁱ and u₀^{o}. Assume further that the object to be detected is represented by the region with the value u₀ⁱ. Let denote its boundary by Co. Then we have u₀≈u₀ⁱ inside the object [or inside (C₀)], and u₀√u₀^{o} outside the object [or outside (C₀)] where μ≧0, v≧0, λ₁, λ₂≧0. In Chan and Vese's approach, λ₁=λ₂=1 and v=0 (T. F. Chan and L. A. Vese. Active Contours Without Edges. IEEE Transactions on Image Processing, 10:266-277, 2001).

The level set functions are defined by equations E16-E19:

$$\begin{matrix}\{ \begin{matrix}C = \partial w = \{ ( x , y ) ∈ Ω \text{:} Φ ( x , y ) = 0 \} \\ inside ( C ) = w = \{ ( x , y ) ∈ Ω \text{:} Φ ( x , y ) > 0 \} \\ outside ( C ) = Ω \backslash \overline{w} = \{ ( x , y ) ∈ Ω \text{:} Φ ( x , y ) < 0 \}\end{matrix} \} & E 16 \\ H ( z ) = \{ \begin{matrix}1 , ( z \geq 0 ) \\ 0 , ( z < 0 )\end{matrix} , {δ}_{0} = \frac{d H ( z )}{d z} . & E 17\end{matrix}$$

The functional may be solved using following equation, E18:

$$F ( {c}_{1} , {c}_{2} , Φ ) = μ {\int }_{Ω} δ ( Φ ( x , y ) ) \vert ∇ Φ ( x , y ) \vert d x d y + v {\int }_{Ω} H ( Φ ( x , y ) ) d x d y + {λ}_{1} {\int }_{inside ( C )} {\vert {u}_{0} ( x , y ) - {c}_{1} \vert}^{2} H ( Φ ( x , y ) ) d x d y + {λ}_{2} {\int }_{outside ( C )} {\vert {u}_{0} ( x , y ) - {c}_{2} \vert}^{2} ( 1 - H ( Φ ( x , y ) ) ) d x d y \qquad E 18$$

And, according to equation E19:

$$\frac{\partial Φ}{\partial t} = δ ( Φ ) [ μ div ( \frac{∇ Φ}{\vert ∇ Φ \vert} ) - v - {{λ}_{1} ( {u}_{0} - {c}_{1} )}^{2} + {{λ}_{2} ( {u}_{0} - {c}_{2} )}^{2} ] . \qquad E 19$$

Image segmentation using shape prior missing or diffuse boundaries is a very challenging problem for medical image processing, which may be due to patient movements, low SNR of the acquisition apparatus or being blended with similar surrounding tissues. Under such conditions, without a prior model to constrain the segmentation, most algorithms (including intensity- and curve-based techniques) fail-mostly due to the under-determined nature of the segmentation process. Similar problems arise in other imaging applications as well and they also hinder the segmentation of the image. These image segmentation problems demand the incorporation of as much prior information as possible to help the segmentation algorithms extract the tissue of interest.

A number of model-based image segmentation algorithms are used to correct boundaries in medical images that are smeared or missing. Alternate embodiments of the segmentation algorithms employ parametric point distribution models for describing segmentation curves. The alternate embodiments include using linear combinations of appearance derived eigenvectors that incorporate variations from the mean shape to correct missing or smeared boundaries, including those that arise from variations in transducer angular viewing or alterations of subject pose parameters. These aforementioned point distribution models are determined to match the points to those having significant image gradients. A particular embodiment employs a statistical point model for the segmenting curves by applying principal component analysis (PCA) in a maximum a-posteriori Bayesian framework that capture the statistical variations of the covariance matrices associated with landmark points within a region of interest. Edge-detection and boundary point correspondence within the image gradients are determined within the framework of the region of interest to calculate segmentation curves under varying poses and shape parameters. The incorporated shape information as a prior model restricts the flow of geodesic active contours so that prior parametric shape models are derived by performing PCA on a collection of signed distance maps of the training shape. The segmenting curve then evolves according to the gradient force of the image and the force exerted by the estimated shape. An “average shape” serves as the shape prior term in their geometric active contour model.

Implicit representation of the segmenting curve has been proposed in and calculated the parameters of the implicit model to minimize the region-based energy based on Mumford-Shah functional for image segmentation. The proposed method gives a new and efficient frame work for segmenting image contaminated by heavy noise and delineating structures complicated by missing or diffuse boundaries.

The shape model training phase of FIG. 23 begins with acquiring a set of training shapes per process block 262. Here a set of binary images {B¹, B², . . . , Bⁿ}, each is with 1 as object and 0 as the background. In order to extract the accurate shape information, alignment is applied. Alignment is a task to calculate the following pose parameters p=[a,b,h,θ]^{T} and correspondingly these four parameters are for translation in x, y, scale and rotation, according to equation E20:

$$T ( p ) = [ \begin{matrix}1 & 0 & a \\ 0 & 1 & b \\ 0 & 0 & 1\end{matrix} ] [ \begin{matrix}h & 0 & 0 \\ 0 & h & 0 \\ 0 & 0 & h\end{matrix} ] [ \begin{matrix}cos ( θ ) & - sin ( θ ) & 0 \\ sin ( θ ) & cos ( θ ) & 0 \\ 0 & 0 & 1\end{matrix} ] \qquad E 20$$

The strategy to compute the pose parameters for n binary images is to use gradient descent method to minimize the special designed energy functional E_{align} for each binary image corresponding to the fixed one, say the first binary image B¹ and the energy is defined as the following equation, according to equation E21:

$${E}_{align}^{j} = \frac{\int {\int }_{Ω} {( {\tilde{B}}_{j} - {B}_{1} )}^{2} d A}{\int {\int }_{Ω} {( {\tilde{B}}_{j} + {B}_{1} )}^{2} d A} \qquad E 21$$

where Ω denotes the image domain, \tilde{B}ⱼ denotes the transformed image of Bⱼ based on the pose parameters p. Minimizing this energy is equivalent to minimizing the difference between current binary image and the fixed image in the training database. The normalization term in the denominator is employed to prevent the images from shrinking to alter the cost function. Hill climbing or Rprop method could be applied for the gradient descent.

FIG. 30 illustrates a 12-panel outline of a left ventricle determined by an experienced sonographer overlapped before alignment by gradient decent. The 12-panel images are overlapped via gradient decent into an aligned shape composite per process block 266 of FIG. 23.

FIG. 31 illustrates a 12-panel outline of a left ventricle determined by an experienced sonographer that is overlapped by gradient decent alignment between zero and level set outlines. Once gradient decent alignment has been accomplished per process block 264 of FIG. 23, additional procedures leading to Principle Components Analysis (PCA) may be performed for acquiring implicit parametric shape parameters from which the segmentation phase may be undertaken.

One approach to represent shapes is via point models where a set of marker points is used to describe the boundaries of the shape. This approach suffers from problems such as numerical instability, inability to accurately capture high curvature locations, difficulty in handling topological changes, and the need for point correspondences. In order to overcome these problems, an Eulerian approach to shape representation based on the level set methods could be utilized.

The signed distance function is chosen as the representation for shape. In particular, the boundaries of each of the aligned shapes are embedded as the zero level set of separate signed distance functions {Ψ₁, Ψ₂, . . . , Ψₙ} with negative distances assigned to the inside and positive distances assigned to the outside of the object. The mean level set function describing the shape value parameters Φ defined in process block 272 of FIG. 23 may be applied to the shape database as the average of these signed distance functions of process block 266, can be computed as shown in equation E22:

$$\overline{Φ} = \frac{1}{n} {\sum }_{i = 1}^{n} {Ψ}_{i} . \qquad E 22$$

To extract the shape variabilities, Φ̅ 21 is subtracted from each of the n signed distance functions to create n mean-offset functions {\tilde{Ψ}₁, \tilde{Ψ}₂, . . . , \tilde{Ψ}ₙ}. These mean-offset functions are analyzed and then used to capture the variabilities of the training shapes.

Specifically, n column vectors are created, \tilde{ψ}ᵢ, from each \tilde{Ψ}ᵢ. A natural strategy is to utilize the N₁×N₂ rectangular grid of the training images to generate N=N₁×N₂ lexicographically ordered samples (where the columns of the image grid are sequentially stacked on top of one other to form one large column). Next, define the shape-variability matrix S as: S={\tilde{ψ}₁, \tilde{ψ}₂, . . . , \tilde{ψ}ₙ}.

FIG. 32 illustrates the procedure for creation of a matrix S of a N₁×N₂ rectangular grid. From this grid an eigenvalue decomposition is employed as shown in equation E23:

$$\frac{1}{n} {SS}^{T} = U Σ {U}^{T} \qquad E 23$$

Here U is a matrix whose columns represent the orthogonal modes of variation in the shape Σ is a diagonal matrix whose diagonal elements represent the corresponding nonzero eigenvalues. The N elements of the ith column of U, denoted by Uᵢ, are arranged back into the structure of the N₁×N₂ rectangular image grid (by undoing the earlier lexicographical concatenation of the grid columns) to yield Φᵢ, the ith principal mode or eigenshape. Based on this approach, a maximum of n different eigenshapes {Φ₁, Φ₂, . . . , Φₙ} are generated. In most cases, the dimension of the matrix 1/nSS^{T} is large so the calculation of the eigenvectors and eigenvalues of this matrix is computationally expensive. In practice, the eigenvectors and eigenvalues of 1/nSS^{T} can be efficiently computed from a much smaller n×n matrix W given by 1/nS^{T}S. It is straightforward to show that if d is an eigenvector of W with corresponding eigenvalue λ, then 1/nSS^{T} is an eigenvector of n with eigenvalue λ.

For segmentation, it is not necessary to use all the shape variabilities after the above procedure. Let k≦n, which is selected prior to segmentation, be the number of modes to consider. k may be chosen large enough to be able to capture the main shape variations present in the training set.

FIG. 33 illustrates a 12-panel training eigenvector image set generated by distance mapping per process block 268 to extract mean eigen shapes.

FIG. 34 illustrates the 12-panel training eigenvector image set wherein ventricle boundary outlines are overlapped.

The corresponding eigenvalues for the 12-panel training images from FIG. 33 are 1054858.250000, 302000.843750, 139898.265625, 115570.250000, 98812.484375, 59266.875000, 40372.125000, 27626.216797, 19932.763672, 12535.892578, 7691.1406, and 0.000001.

From these shapes and values the shape knowledge for segmentation is able to be determined via a new level set function defined in equation E24:

$$Φ [ w ] ( x , y ) = \overline{Φ} ( \tilde{x} , \tilde{y} ) + {\sum }_{i = 1}^{k} {w}_{i} {Φ}_{i} ( \tilde{x} , \tilde{y} ) \qquad E 24$$

Here w={w₁, w₂, . . . , wₖ} are the weights for the k eigenshapes with the variances of these weights {σ₁², σ₂², . . . , σₖ²} given by the eigenvalues calculated earlier. Now we can use this newly constructed level set function Φ as the implicit representation of shape as shape values. Specifically, the zero level set of Φ describes the shape with the shape's variability directly linked to the variability of the level set function. Therefore, by varying w, Φ can be changed which indirectly varies the shape. However, the shape variability allowed in this representation is restricted to the variability given by the eigenshapes.

FIG. 35 illustrated the effects of using different w or k-eigenshapes to control the appearance and newly generated shapes. Here one shape generates a 6-panel image variation composed of three eigen value pairs in +1 and −1 signed values.

The segmentation shape modeling of FIG. 23 begins with process block 270 to undergo addition processes to account for shape variations or differences in poses. To have implicit representation the flexibility of handling pose variations, p is added as another parameter to the level set function according to equation E25:

$$Φ [ w , p ] ( x , y ) = \overline{Φ} ( \tilde{x} , \tilde{y} ) + {\sum }_{i = 1}^{k} {w}_{i} {Φ}_{i} ( \tilde{x} , \tilde{y} ) \qquad E 25$$

As a segmentation using shape knowledge, the task is to calculate the w and pose parameters p. The strategy for this calculation is quite similar as the image alignment for training. The only difference is the special defined energy function for minimization. The energy minimization is based on Chan and Vese's active model (T. F. Chan and L. A. Vese. Active contours without edges. IEEE Transactions on Image Processing, 10: 266-277, 2001) as defined by following equations E26-E35:

$$\begin{matrix}{R}^{u} = \{ ( x , y ) ∈ {R}^{2} \text{:} Φ ( x , y < 0 ) \} \\ {R}^{v} = \{ ( x , y ) ∈ {R}^{2} \text{:} Φ ( x , y > 0 ) \} & E 26 \\ area in & \\ {R}^{u} \text{:} {A}_{u} = \int {\int }_{Ω} H ( - Φ [ w , p ] ) d A & E 27 \\ area in & \\ {R}^{v} \text{:} {A}_{v} = \int {\int }_{Ω} H ( Φ [ w , p ] ) d A & E 28 \\ sum intensity in & \\ {R}^{u} \text{:} {S}_{u} = \int {\int }_{Ω} IH ( - Φ [ w , p ] ) d A & E 29 \\ sum intensity in & \\ {R}^{v} \text{:} {S}_{v} = \int {\int }_{Ω} IH ( Φ [ w , p ] ) d A & E 30 \\ average intensity in & \\ {R}^{u} \text{:} μ = \frac{{S}_{u}}{{A}_{u}} & E 31 \\ average intensity in & \\ {R}^{v} \text{:} γ = \frac{{S}_{v}}{{A}_{v}} & E 32 \\ where & \\ H ( Φ [ w , p ] ) = \{ \begin{matrix}1 , if Φ [ w , p ] \geq 0 \\ 0 if Φ [ w , p ] < 0\end{matrix} & E 33 \\ {E}_{cv} = {\int }_{{R}^{u}} {( I - μ )}^{2} d A + {\int }_{{R}^{v}} {( I - v )}^{2} d A & E 34 \\ {E}_{cv} = - ( {μ}^{2} {A}_{u} + {v}^{2} {A}_{v} ) = - ( \frac{{S}_{u}^{2}}{{A}_{u}} + \frac{{S}_{v}^{2}}{{A}_{v}} ) & E 35\end{matrix}$$

The definition of the energy could be modified for specific situation. In a particular embodiment, the design of the energy includes the following factors in addition to the average intensity, the standard deviation of the intensity inside the region.

Once the 3D volume image data could be reconstructed, a 3D shape model could also be defined in other particular embodiments having modifications of the 3D signed distance, the Degrees of Freedom (DOFs) (for example the DOF could be changed to nine, including transition in x, y, z, rotation α, β, θ, scaling factor sx, sy, sz), and modifications of the principle component analysis (PCA) to generate other decomposition matrixes in 3D space. One particular embodiment for determining the heart chamber ejection fractions is also to access how the 3D space could be affected by 2D measurements obtained over time for the same real 3D volume.

FIG. 36 is an image of variation in 3D space affected by changes in 2D measurements over time. Presented are three views of 2D+time echocardiographic data collected by transceivers 10A-E. The images are based on 24 frames taken at different time points, has a scaling factor in time dimension as 10 and is tri-linear interpolated in a 3D data set with pixel size as 838 by 487 by 240.

FIG. 37 is a 7-panel phantom training image set compared with a 7-panel aligned set. The left column are original 3D training data set in three views and the right column is a 7-panel image set of the original 3D training data set after alignment in three views. The phantom is synthesized as a simulation for the 2D+time echocardiographic data.

FIG. 38 is a phantom training set comprising variations in shapes. The left 3-panel column presenting an average shape −0.5 variation, the right 3-panel column presenting an average shape +0.5 variation, the middle image with overlapping crosshairs represents the average extracted shape from the phantom measurements.

FIG. 39 illustrates the restoration of properly segmented phantom measured structures from an initially compromised image using the aforementioned particular image training and segmentation embodiments. The top image has two differently sized and shaped hourglasses and an oval that is lacks boundary delineation. The second image from the top depicts the initial position of the average shape in the original 3D image, which is presented in a white outline and is off-center from the respective shapes. The second image from the top depicts the final segmentation result but still off-centered. The bottom image depicts a comparison between manual segmentation and automated segmentation. Here there is virtual overlap and shape alignment for the manually segmented and the automatic segmented shapes.

FIG. 40 schematically depicts a particular embodiment to determine shape segmentation of a ROI. An ROI is defined and gives the initialization of the shape based segmentation. The mass area (shown in light shadow), center, and longest axis of the ROI are computed. There after, the area of ROI is determined of to help decide the initial scaling factor. The scaling factor is defined as the square root of the quotient of the ROI area and the area's average shape. The direction of the longest axis (theta based on the y-axis) is used to determine the initial rotational angle. The center of the mass determines the initial transition in the x and in y-axes. Thereafter the detected shadow is used to remove the interference from the non-LV region and the average contour from training system on the mass center is computed from the ROI into a created object sub image. The region based segmentation within the sub region is undertaken by the aforementioned method particular embodiments.

FIG. 41 illustrates an exemplary transthoracic apical view of two heart chambers. The hand-held transceivers 10A-D substantially captures two chambers of a heart (outlined in dashed line) within scan plane 42. The two chamber view within the single scan plane 42 of a 3D dataset is collected at maximum mitral valve centering as described for FIG. 8 by procedures undertaken in sub-algorithm 280 of FIG. 24.

FIG. 42 illustrates other exemplary transthoracic apical views as panel sets associated with different rotational scan plane angles. The panel sets illustrated are associated with rotational scan planes θ angles 0, 30, 60 and 90 degrees.

FIG. 43 illustrates a left ventricle segmentation from different weight values w applied to a panel of eigenvector shapes. Here a panel of three eigenvectors pairs are weighted at w=+1 and w=−1 for a total of six segmentation shapes. The mean or average model segmentation shape from the six-segmented shapes is shown.

FIG. 44 illustrates exemplary Left Ventricle segmentations using the trained level-set algorithms. The segmentations are from a collection of 2D scan planes contained within a 3D data set acquired during an echocardiographic procedure in particular embodiments previously described by the systems illustrated in FIGS. 12-19 and methods in FIGS. 20-25. Scan planes are 30, 60, and 90 degrees and show the original image, the image as resulting from procedures having some computational cost (Inverted with histogram equalization), the original image modified with sonographer-overlaid segmentation, the original image modified by the computational cost and initial average segmented shape associated with the trained level-set algorithms, and final average segmented shape as determined by the trained level-set algorithms. Other echocardiographic particular embodiments may obtain initial and final segmentation as determined by the trained level-set algorithms under a 2D+ time analysis image acquisition mode to more readily handle pose variations described above and to compensate for segmentation variation and the correspondingly Left ventricle area variation arising during movement of the heart while beating.

Validation data for determining volumes of heart chambers using the level-set algorithms is shown in the Table 1 for 33 data sets and compared with manual segmentation values. For each angle, there are 24 time-based that provide 864 2D-segmentations (=24×36).

TABLE 1

| | | | | | | | | Total |
|---|---|---|---|---|---|---|---|---|
| | | | | | | | | frames |
| | | | | | | | | (Data |
| | 1002 | 1003 | 1006 | 1007 | 1012 | 1016 | 1017 | sets) |
| Angle 0 | | ✓ | ✓ | ✓ | ✓ | ✓ | ✓ | 144 (6) |
| Angle 30 | ✓ | ✓ | ✓ | ✓ | ✓ | ✓ | ✓ | 168 (7) |
| Angle 60 | ✓ | ✓ | | ✓ | ✓ | ✓ | ✓ | 144 (6) |
| Angle 90 | | ✓ | ✓ | ✓ | ✓ | ✓ | ✓ | 144 (6) |
| Angle 300 | | | ✓ | ✓ | ✓ | ✓ | ✓ | 120 (5) |
| Angle 330 | ✓ | | ✓ | ✓ | ✓ | ✓ | ✓ | 144 (6) |
| | | | | | | | | 864 (36) |

The manual segmentation is stored in .txt file, in which the expert-identified landmarks are stored. The .txt file is with the name as following format: ****-XXX-outline.txt where **** is the data set number and XXX is the angle. Table 2 below details segmentation results by the level-set algorithms. When these landmarks are used for segmentation, linear interpolation may be used to generate closed contour.

TABLE 2

| | Level-set | Level-set | |
|---|---|---|---|
| Sono- | algorithm | algorithm | |
| grapher- | determined | determined | Time stamp |
| located | X-axis landmark | Y-axis landmark | (frame number) for |
| landmark | location | location | landmark placement |
| 1 | 395 | 313 | 1 |
| 2 | 380 | 312 | 1 |
| 41 | 419 | 315 | 1 |
| 42 | 407 | 307 | 2 |
| 73 | 446 | 304 | 2 |
| 74 | 380 | 301 | 3 |
| 110 | 459 | 295 | 3 |
| 860 | 435 | 275 | 24 |

Training the level-set algorithm's segmentation methods to recognize shape variation from different data sets having different phases and/or different viewing angles is achieved by processing data outline files. The outline files are classified into different groups. For each angle within the outline files, the corresponding outline files are combined into a single outline file. At the same time, another outline file is generated including all the outline files. Segmentation training also involves several schemes. The first scheme trains part of the segmentation for each data set (fixed angle). The second scheme trains via the segmentation for fixed angle from all the data sets. The third scheme trains via the segmentation for all the segmentation for all angles.

For a validation study 75-2D segmentation results were selected from 3D datasets collected for different angles from Table 1. The angles randomly selected are 1002 1003 1007 1016.

Validation methods include determining positioning, area errors, volume errors, and/or ejection fraction errors between the level-set computer readable medium-generated contours and the sonographer-determined segmentation results. Area errors of the 2D scan use the following definitions: A denotes the automatically-identified segmentation area, M the manually-identified segmentation area determined by the sonographer. Ratios of overlapping areas were assessed by applying the similarity Kappa index (KI) and the overlap index, which are defined as:

$$KI = 2 \times \frac{A ⋂ M}{A + M} overlap = \frac{A ⋂ M}{A ⋃ M}$$

Volume error: (3D) After 3D reconstruction, the volume of the manual segmentation and automated segmentation are compared using the similar validation indices as the area error.

Ejection fraction (EF) error in 4D (2D+time) is computed using the 3D volumes at different heart phases. The EF from manual segmentation with the EF from auto segmentation are compared.

Results: The training is done using the first 12 images for the 4 different angles of data set 1003. Collected training sets for 4 different angles, 0, 30, 60 and 90 are created. The segmentation was done for the last 12 image for the 4 different angles of data set 1003. Subsequently, the segmentation for 4 different angles, 0, 30, 60 and 90 degrees was collected and are respectively presented in Tables 3-6 below.

TABLE 3
(angle 1003-000):

| | unsigned | signed | | | | | |
|---|---|---|---|---|---|---|---|
| | positioning | positioning | Auto | Manual | Overlapping | | |
| | error | error | area | area | area | KI | Overlapping |
| Data | (in mms) | (in mms) | (in mms) | (in mms) | (in mms) | 2*O/(A + M) | O/(AorM) |
| frame | 3.661566 | 3.344212 | 2788.387 | 2174.345486 | 2138.234448 | 0.861717 | 0.757032 |
| 13 | | | | | | | |
| frame | 3.634772 | 3.222219 | 2918.387 | 2299.888968 | 2250.409162 | 0.862511 | 0.758258 |
| 14 | | | | | | | |
| frame | 3.406509 | 2.938201 | 2953.883 | 2395.160643 | 2336.000006 | 0.873427 | 0.775296 |
| 15 | | | | | | | |
| frame | 6.847305 | 6.658746 | 3041.164 | 1764.52362 | 1743.471653 | 0.725587 | 0.56935 |
| 16 | | | | | | | |
| frame | 5.696813 | 5.554389 | 2853.694 | 1813.849761 | 1796.793058 | 0.769909 | 0.625897 |
| 17 | | | | | | | |
| frame | 3.570965 | 2.28045 | 3001.365 | 2533.919227 | 2414.983298 | 0.872578 | 0.773958 |
| 18 | | | | | | | |
| frame | 3.819476 | 2.335655 | 2909.474 | 2486.437054 | 2312.028423 | 0.856956 | 0.749713 |
| 19 | | | | | | | |
| frame | 4.694806 | 2.774984 | 3149.651 | 2741.058289 | 2482.44179 | 0.842833 | 0.728359 |
| 20 | | | | | | | |
| frame | 3.422007 | 2.055935 | 2848.469 | 2498.730173 | 2321.555591 | 0.868326 | 0.767293 |
| 21 | | | | | | | |
| frame | 6.691374 | 6.41405 | 2994.45 | 1804.783586 | 1773.743459 | 0.739178 | 0.586266 |
| 22 | | | | | | | |
| frame | 4.787031 | 4.286448 | 2901.483 | 2126.555985 | 2064.629396 | 0.766664 | 0.621618 |
| 23 | | | | | | | |
| frame | 4.724921 | 3.749576 | 2895.337 | 2303.576904 | 2174.49915 | 0.836521 | 0.718982 |
| 24 | | | | | | | |
| | | Max Area | 3149.651 | 2741.058289 | | | |
| | | (ED—end- | | | | | |
| | | diastolic) | | | | | |
| | | Min Area | 2788.387 | 1764.52362 | | | |
| | | (ES—end- | | | | | |
| | | systolic) | | | | | |
| | | EF (1 − | | | | | |
| | | ES/ED) | | | | | |
| ave | 4.579795417 | 3.80123875 | | | | 0.8230173 | 0.7026685 |
| std | 1.24222826 | 1.5997941 | | | | 0.0558833 | 0.0783376 |

FIG. 45 is a plot of the level-set automated left ventricle area vs. the sonographer manually measured area of angle 1003-000 from Table 3.

TABLE 4
(angle 1003-030):

| Data | unsigned | signed | | | | | |
|---|---|---|---|---|---|---|---|
| Data | positioning | positioning | Auto | Manual | Overlapping | | |
| 1003- | error | error | area | area | area | KI | Overlapping |
| 030 | (in mms) | (in mms) | (in mms) | (in mms) | (in mms) | 2*O/(A + M) | O/(AorM) |
| frame | 2.19382 | 2.160323 | 3308.847 | 2799.60427 | 2795.76267 | 0.915375 | 0.843956 |
| 13 | | | | | | | |
| frame | 0.870204 | −0.104675 | 3252.145 | 3293.019348 | 3163.634267 | 0.966709 | 0.935563 |
| 14 | | | | | | | |
| frame | 2.714477 | 0.575919 | 2761.496 | 2686.353907 | 2422.820161 | 0.889459 | 0.800925 |
| 15 | | | | | | | |
| frame | 5.183792 | 4.942926 | 2718.162 | 1771.438499 | 1735.020133 | 0.772906 | 0.629867 |
| 16 | | | | | | | |
| frame | 2.641074 | −1.125789 | 2690.964 | 3002.133411 | 2532.382588 | 0.889633 | 0.801206 |
| 17 | | | | | | | |
| frame | 1.882148 | 0.187478 | 3122.145 | 3104.012638 | 2886.578089 | 0.927242 | 0.864354 |
| 18 | | | | | | | |
| frame | 1.934285 | −0.736144 | 3156.412 | 3373.231952 | 3018.729122 | 0.924623 | 0.859813 |
| 19 | | | | | | | |
| frame | 2.289288 | −1.470268 | 2713.245 | 3078.350751 | 2625.656631 | 0.906713 | 0.829345 |
| 20 | | | | | | | |
| frame | 3.722941 | −0.242956 | 2596.921 | 2725.077233 | 2240.881995 | 0.906073 | 0.84212 |
| 21 | | | | | | | |
| frame | 4.493668 | 2.607496 | 2543.293 | 2092.903571 | 1880.232606 | 0.81111 | 0.682241 |
| 22 | | | | | | | |
| frame | 2.40633 | 0.700761 | 2642.252 | 2522.0871 | 2313.718727 | 0.896037 | 0.811654 |
| 23 | | | | | | | |
| frame | 2.22815 | −1.754062 | 2514.097 | 2971.554277 | 2466.614399 | 0.899297 | 0.81702 |
| 24 | | | | | | | |
| | | Max Area | 3308.847 | 3373.231952 | | | |
| | | (ED—end- | | | | | |
| | | diastolic) | | | | | |
| | | Min Area | 2514.097 | 1771.438499 | | | |
| | | (ES—end- | | | | | |
| | | systolic) | | | | | |
| | | EF (1 − | | | | | |
| | | ES/ED) | | | | | |
| ave | 2.760577909 | 0.325516909 | | | | 0.889982 | 0.806737091 |
| std | 1.244070529 | 1.950425351 | | | | 0.053912517 | 0.084541431 |

FIG. 46 is a plot of the level-set automated left ventricle area vs. the sonographer manually measured area of angle 1003-030 from Table 4.

TABLE 5
(angle 1003-060):

| | unsigned | signed | | | | | |
|---|---|---|---|---|---|---|---|
| | positioning | positioning | Auto | Manual | Overlapping | | |
| | error | error | area | area | area | KI | Overlapping |
| Data | (in mms) | (in mms) | (in mms) | (in mms) | (in mms) | 2*O/(A + M) | O/(AorM) |
| frame | 5.402612 | 1.131598 | 2095.055 | 2077.384 | 1627.455339 | 0.780098 | 0.639476 |
| 13 | | | | | | | |
| frame | 6.067347 | 0.724424 | 1892.987 | 1996.556 | 1431.533749 | 0.736094 | 0.582396 |
| 14 | | | | | | | |
| frame | 4.970993 | 1.225224 | 2686.508 | 2607.524 | 2157.749775 | 0.815163 | 0.687996 |
| 15 | | | | | | | |
| frame | 5.421482 | 1.441104 | 2499.498 | 2455.858 | 1950.149722 | 0.787088 | 0.648924 |
| 16 | | | | | | | |
| frame | 5.145954 | −1.543341 | 2247.182 | 2750.893 | 1954.452314 | 0.782082 | 0.642147 |
| 17 | | | | | | | |
| frame | 5.2217 | 0.651928 | 2267.312 | 2343.53 | 1813.388769 | 0.786576 | 0.648229 |
| 18 | | | | | | | |
| frame | 7.271475 | 1.621387 | 1998.861 | 2074.464 | 1420.623606 | 0.697525 | 0.535538 |
| 19 | | | | | | | |
| frame | 6.651073 | 2.366935 | 2334.002 | 2204.156 | 1653.578218 | 0.728744 | 0.573247 |
| 20 | | | | | | | |
| frame | 6.598955 | 1.980833 | 2708.943 | 2615.361 | 2013.151959 | 0.756212 | 0.607991 |
| 21 | | | | | | | |
| frame | 5.943021 | 1.79845 | 2591.082 | 2530.385 | 1988.104728 | 0.776381 | 0.634496 |
| 22 | | | | | | | |
| frame | 5.499417 | −1.160939 | 2336.154 | 2682.205 | 1942.620186 | 0.774205 | 0.631595 |
| 23 | | | | | | | |
| frame | 6.543109 | 1.373915 | 2343.53 | 2379.641 | 1767.135908 | 0.748284 | 0.597806 |
| 24 | | | | | | | |
| | | Max Area | 2708.943 | 2750.893 | | | |
| | | (ED—end- | | | | | |
| | | diastolic) | | | | | |
| | | Min Area | 1892.987 | 1996.556 | | | |
| | | (ES—end- | | | | | |
| | | systolic) | | | | | |
| | | EF (1 − | | | | 0.762578 | 0.617306 |
| | | ES/ED) | | | | | |
| ave | 5.939502364 | 0.95272 | | | | 0.033122 | 0.042875 |
| std | 0.751069178 | 1.246998298 | | | | | |

FIG. 47 is a plot of the level-set automated left ventricle area vs. the sonographer manually measured area of angle 1003-060 from Table 5.

TABLE 6
(angle 1003-090):

| | unsigned | signed | | | | | |
|---|---|---|---|---|---|---|---|
| | positioning | positioning | Auto | Manual | Overlapping | | |
| | error | error | area | area | area | KI | Overlapping |
| Data | (in mms) | (in mms) | (in mms) | (in mms) | (in mms) | 2*O/(A + M) | O/(AorM) |
| frame | 4.890372 | 0.386783 | 2791.767 | 2897.181 | 2341.993 | 0.823348 | 0.699738 |
| 13 | | | | | | | |
| frame | 4.845072 | −0.237482 | 2580.479 | 2835.562 | 2206.461 | 0.814787 | 0.687461 |
| 14 | | | | | | | |
| frame | 2.913541 | −2.216814 | 2590.007 | 3139.509 | 2531.992 | 0.883817 | 0.791821 |
| 15 | | | | | | | |
| frame | 9.910783 | 8.934044 | 3650.903 | 2067.549 | 1931.71 | 0.675606 | 0.510125 |
| 16 | | | | | | | |
| frame | 6.945058 | 4.461438 | 2608.907 | 2072.927 | 1763.448 | 0.753315 | 0.604254 |
| 17 | | | | | | | |
| frame | 3.467966 | 2.314185 | 3071.897 | 2660.231 | 2512.406 | 0.876605 | 0.780318 |
| 18 | | | | | | | |
| frame | 3.62614 | 1.123661 | 2676.673 | 2524.392 | 2233.352 | 0.858806 | 0.75255 |
| 19 | | | | | | | |
| frame | 3.831596 | −0.535588 | 2537.146 | 2803.139 | 2241.65 | 0.839525 | 0.723432 |
| 20 | | | | | | | |
| frame | 3.344675 | 0.791006 | 2541.756 | 2517.631 | 2161.13 | 0.854305 | 0.745666 |
| 21 | | | | | | | |
| frame | 4.183485 | 2.231353 | 2580.94 | 2266.237 | 2037.738 | 0.840794 | 0.725319 |
| 22 | | | | | | | |
| frame | 3.734046 | 3.58284 | 3136.436 | 2424.818 | 2405.917 | 0.865243 | 0.762491 |
| 23 | | | | | | | |
| frame | 3.189541 | 1.353026 | 2840.479 | 2604.451 | 2391.165 | 0.878309 | 0.783022 |
| 24 | | | | | | | |
| | | Max Area | 3650.903 | 3139.509 | | | |
| | | (ED—end- | | | | | |
| | | diastolic) | | | | | |
| | | Min Area | 2537.146 | 2067.549 | | | |
| | | (ES—end- | | | | | |
| | | systolic) | | | | | |
| | | EF (1 − | | | | | |
| | | ES/ED) | | | | | |
| ave | 4.544718455 | 1.981969909 | | | | 0.83101 | 0.715133 |
| std | 2.094832841 | 2.977565291 | | | | 0.063412 | 0.086357 |

FIG. 48 is a plot of the level-set automated left ventricle area vs. the sonographer or manually measured area of angle 1003-090 from Table 6.

Using the trained algorithms applied to the 3D data sets from the 3D transthoracic echocardiograms shows that these echocardiographic systems and methods provide powerful tools for diagnosing heart disease. The ejection fraction as determined by the trained level-set algorithms to the 3D datasets provides an effective, efficient and automatic measurement technique. Accurate computation of the ejection fractions by the applied level-set algorithms is associated with the segmentation of the left ventricle from these echocardiography results and compares favorably to the manually, laboriously determined segmentations.

The proposed shape based segmentation method makes use of the statistical information from the shape model in the training datasets. On one hand, by adjusting the weights for different eigenvectors, the method is able to match the object to be segmented with all different shape modes. On the other hand, the topology-preserving property can keep the segmentation from leakage which may be from the low quality echocardiography.

FIG. 49 illustrates the 3D-rendering of a portion of the Left Ventricle from 30 degree angular view presented from six scan planes obtained at systole and/or diastole. Here the planar shapes of a 12-panel 2D image set are rendered to provide a portion of the Left Ventricle as a combined 3D rendering of systole and/or diastole measurements. More particularly, the upper image set encompasses 2D views of the left ventricle at different heart phases and overlapped with the segmentation results of the images contained in the six scan planes acquired at the 30-degree locus. The lower image indicates the range of motion of the left ventricular endocardium between systole and diastole viewable from the 30-degree locus from the segmentated 2D images of the six scan planes.

Left Ventricular Mass (LVM): LV hypertrophy, as defined by echocardiography, is a predictor of cardiovascular risk and higher mortality. Anatomically, LV hypertrophy is characterized by an increase in muscle mass or weight. LVM is mainly determined by two factors: chamber volume, and wall thickness. There are two main assumptions in the computation of LVM: 1) the interventricular septum is assumed to be part of the LV and 2) the volume, Vₘ, of the myocardium is equal to the total volume contained within the epicardial borders of the ventricle, Vₜ(epi), minus the chamber volume, V_{c}(endo); Vₘ is defined by equation E36 and LVM is obtained by multiplying Vₘ by the density of the muscle tissue (1.05 g/cm) according to E37:

Vₘ=Vₜ(epi)−V_{c}(endo) E36

LVM=1.05×Vₘ E37

LVM is usually normalized to total body surface area or weight in order to facilitate interpatient comparisons. Normal values of LVM normalized to body weight are 2.4±0.3 g/kg [42].

Stroke Volume (SV): is defined as the volume ejected between the end of diastole and the end of systole as shown in E38:

SV=end_diastolic_volume(EDV)−end_systolic_volume(ESV) E38

Alternatively, SV can be computed from velocity-encoded MR images of the aortic arch by integrating the flow over a complete cardiac cycle [54]. Similar to LVM and LVV, SV can be normalized to total body surface. This corrected SV is known as SVI (Stroke volume index). Healthy subjects have a normal SVI of 45±8 ml/m [42].

Ejection Fraction (EF): is a global index of LV fiber shortening and is generally considered as one of the most meaningful measures of the LV pump function. It is defined as the ratio of the SV to the EDV according to E39:

$$EF = \frac{SV}{EDV} \times 100 \% = \frac{EDV - ESV}{EDV} \times 100 \% \qquad E 39$$

Cardiac Output (CO): The role of the heart is to deliver an adequate quantity of oxygenated blood to the body. This blood flow is known as the cardiac output and is expressed in liters per minute. Since the magnitude of CO is proportional to body surface, one person may be compared to another by means of the CT, that is, the CO adjusted for body surface area. Lorenz et al. [42] reported normal CT values of 2.9±0.6 l/min/m and a range of 1.74-4.03 l/min/m.

CO was originally assessed using Fick's method or the indicator dilution technique [55]. It is also possible to estimate this parameter as the product of the volume of blood ejected within each heart beat (the SV) and the HR according to E40:

CO=SV×HR E40

In patients with mitral or aortic regurgitation, a portion of the blood ejected from the LV regurgitates into the left atrium or ventricle and does not enter the systemic circulation. In these patients, the CO computed with angiocardiography exceeds the forward output. In patients with extensive wall motion abnormalities or misshapen ventricles, the determination of SV from angiocardiographic views can be erroneous. Three-dimensional imaging techniques provide a potential solution to this problem since they allow accurate estimation of the irregular LV shape.

FIG. 50 illustrates four images which are the training results from a larger training set. The four images are respectively, left to right, overlapping before alignment, overlapping after alignment, average level set, and zero level set of the average map respectively.

FIG. 51 illustrates a total of 16 shape variations with differing W values. The W values, left to right, are respectively, −0.2, −0.1, +0.1, and +0.2.

FIG. 52 presents an image result showing boundary artifacts of a left ventricle that arises by employing the estimate shadow regions algorithm 234 of FIG. 22. An original scan plane image on the upper left panel shows a left ventricle LV. The estimate shadow regions 234 processing block provides a negative 2-tone image of the left ventricle and shows potential segmentation complexities exhibited as two spikes Sₐ and S_{b} in the upper right panel image along the boundary of the left ventricle. An area fill is shown in the lower left panel image. A shadow of the original image panel is shown in the lower right image panel.

FIG. 53 illustrates a panel of exemplary images showing the incremental effects of application of level-set sub-algorithm 260 of FIG. 23. The upper left image is a portion of an original image of a Left Ventricle of a scan plane. The upper right is the original plus initial shape segmentation of the level-set algorithm obtained from process block 270 of sub-algorithm 260. The lower left image is the final segmentation result of the trained level-set algorithm exiting from processing block 276 of sub-algorithm 260. The lower right image is the sonographer determined segmentation. As can be seen the final trained level-set algorithm compares favorably with the manually segmented result of the sonographer.

FIG. 54 illustrates another panel of exemplary images showing the incremental effects of application of an alternate embodiment of the level-set sub-algorithm 260 of FIG. 23. The upper left image an original image of a Left Ventricle of a scan plane. The upper right is an inverse or negative two-tone image of the original. The middle left image is the original image masked with shadow. The middle right is the original plus initial shape segmentation of the level-set algorithm obtained from process block 270 of sub-algorithm 260. The lower left image is the final segmentation result of the trained level-set algorithm exiting from processing block 276 of sub-algorithm 260. The lower right image is the sonographer-determined segmentation. With this alternate level-set algorithm embodiment, it can be seen that the final trained level-set algorithm compares favorably with the manually segmented result of the sonographer.

FIG. 55 presents a graphic of Left Ventricle area determination as a function of 2D segmentation with time (2D+time) between systole and diastole by application of the particular and alternate embodiments of the level set algorithms of FIG. 23. As can be seen, the Left ventricle area presents a sinusoidal repetition and shows that both the particular embodiment of the automatic level-set algorithm of FIGS. 23 and 53 and the alternate embodiment described in FIG. 54 presents a favorable accuracy with the manual sonographer segmentation methods of FIGS. 21 and 22. The automatic level-set particular and alternate embodiments present segmentation areas substantially the same as the fully manual sonographer method across the range between diastole and/or systole.

FIGS. 56-58 collectively illustrates Bayesian inferential approaches to segmentation described by Mikael Rousson and Daniel Cremers in Efficient Kernel Density Estimation of Shape and Intensity Priors for Level Set Segmentation (MICCAI (2) 2005: 757-764). The complexities for determining organ boundary information from boundary-specific echogenic signals that is mixed with noise and background overlap from neighboring structures. By way of example, FIG. 56 illustrates the empirical probability of intensities inside and outside the left ventricle of an ultrasound cardio image. The echogenic intensity of the internal surface (dashed line) significantly overlaps with the echogenic intensity (solid line) of external surfaces of the left ventricle. The region-based segmentation of these structures is a challenging problem, because objects and background have similar histograms. The proposed segmentation scheme optimally exploits the estimated probabilistic intensity models in the Bayesian interface.

FIG. 57 depicts three panels in which schematic representations of a curved shaped eigenvector of a portion of a left ventricle is progressively detected when applied under uniform, Gaussian, and Kernel density pixel intensity distributions. The accuracy of segmentation is based on shape model employed and the region information signal intensity. The left frame shows a pattern of points associated in a portion of a scan plane having uniform signal probability densities and no shape. The middle frame shows the same pattern of points associated with an oval shape in which signal intensities are arranged in gaussian probability cluster. The right frame shows the pattern of points associated in a C-shape in the portion of a scan plane having kernel probability densities about the C-shape. The three panels have the same schematic representations of a curved shaped eigenvector of a portion of a left ventricle that is progressively detected when applied under uniform, Gaussian, and Kernel density pixel intensity distributions. A progression of improving resolved eigenshapes is seen from the left to the right panels. The curved-shaped pixel dataset represents a portion of the left ventricle. In the left panel, uniform pixel intensity of a scan plane is applied with the result that no eigen shapes are visible. In the middle panel, a Gaussian pixel intensity distribution is assumed and the curved-shaped pixel sets are contained within an eigen shaped oval pattern. In the right panel, a C-shaped eigenvector is rendered visible that encapsulates the curved pixel data set. That means we are trying to find a for different eigneshapes in the whole a space without any restriction. In the left panel, the a space of signed distance functions is not a linear space, therefore, the mean shape and linear combinations of eigneshapes are typically no longer signed distance functions and cannot be readily seen. In the Gaussian density of the middle panel, a portion of the signed functions allow the curve-shaped data sets to be contained with an oval space. In the right panel the greater proportion of signed functions allow a more certain and improved eigen shape that encompasses the curved-shape data points.

FIG. 58 depicts the expected segmentation of the left ventricle arising from the application of different a-priori model assumptions. In the top panel, a non-model assumption is applied with aberrantly shaped segmented structures that do not render the expected shaped of a left ventricle in that it is jagged and disjointed into multiple chambers. In the middle panel, a prior uniform model assumption is applied, and the left ventricle is partially improved, but does not having the expected shape and is still jagged. In the bottom panel, a prior kernel model is applied to the left ventricle. The resulting segmentation is more cleanly delineated and the ventricle boundary is smooth, has the expected shape, and does not significantly overlap into the inter-chamber wall.

FIG. 59 is a histogram plot of 20 left ventricle scan planes to determine boundary intensity probability distributions employed for establishing segmentation within training data sets of the left ventricle. Maxima for internal and external probability distributions for intensity of pixels residing on the internal or external segmentation line of the left ventricle interface in which pixel intensity along a boundary is compared to the pixel intensity distribution of the whole scan plane image. In the training data sets of a given scan plane, the average pixel intensity probability distribution is calculated and stored with the boundary histograms for segmentation.

FIG. 60 depicts a 20-panel training image set of aligned left ventricle shapes contained in Table 3. Principle component analysis extracts the eigenmodes from each left ventricle image and applies a kernel function to define the distribution of the shape prior and to acquire the eigenvectors obtained from the level-set algorithms described above. Table 6 lists vectors representing each training shape of four eigenmodes to represent the new shape or training shape. Each row represents the vector that corresponds to the training shape. The weights of each training shape are computed by projection to the basis formed by the eigenshapes.

TABLE 6
20 4

| | | | |
|---|---|---|---|
| −0.108466 | −0.326945 | −0.011603 | −0.270630 |
| 0.201111 | 0.007938 | 0.205365 | −0.157808 |
| −0.084072 | −0.127009 | 0.110204 | −0.248149 |
| −0.004642 | 0.018199 | −0.201792 | −0.221856 |
| −0.055033 | −0.262811 | −0.324424 | −0.225715 |
| 0.210304 | 0.007946 | 0.000766 | 0.187720 |
| −0.219551 | −0.326738 | 0.195884 | 0.070594 |
| −0.204191 | 0.218314 | 0.000759 | 0.224303 |
| 0.066532 | −0.499781 | 0.037092 | 0.228500 |
| −0.461649 | −0.178653 | −0.316081 | 0.040002 |
| −0.383818 | −0.380613 | −0.140760 | 0.030318 |
| 0.005501 | 0.004479 | 0.018898 | 0.182005 |
| −0.194213 | 0.008519 | 0.017103 | 0.008163 |
| −0.453880 | 0.134978 | 0.037047 | 0.213359 |
| 0.191661 | −0.004739 | −0.003520 | −0.021242 |
| −0.278152 | 0.251390 | −0.500381 | 0.050353 |
| −0.480242 | −0.215070 | −0.161644 | 0.058304 |
| −0.114089 | 0.228670 | 0.284464 | 0.065447 |
| 0.062613 | 0.289096 | 0.113080 | −0.064892 |
| −0.646280 | −0.035933 | 0.089240 | −0.423474 |

FIG. 61 depicts the overlaying of the segmented left ventricle to the 20-image panel training set obtained by the application of level set algorithm generated eigen vectors of Table 6. The overlaid ventricle segmentation boundary is substantially reproduced and closely follows the contour of each training image. The vectors obtained by the level set algorithms in conjunction with the kernel function adequately and faithfully reconstruct the segmented boundary of the left ventricle, demonstrating the robustness of the system and methods of the particular embodiments.

FIG. 62 depicts the left ventricle segmentation resulting from application of a prior uniform shape statistical model. The prior uniform shape model employs level set trained algorithms applied to information contained in cardiographic echoes. The segmentation results of a subject's left ventricle boundary renders a jagged and spiked left ventricle with overlap into adjacent wall structures.

FIG. 63 depicts the segmentation results of a kernel shape statistical model applied to the echogenic image information of the subject's left ventricle. In the kernel model, the level set trained algorithms results in a smoother segmentation of expected shape without overlap into adjacent wall structures. The application of the kernel shape model with the level set trained algorithms obtained this higher resolving segmentation in only 0.13 seconds due to the fast processing speeds imparted by the level-set algorithms. Thus, the subject's left ventricle segmented shape is efficiently and robustly obtained with high resolution.

The application of the trained level set algorithms with the kernel shape model allows accurate 3D cardiac functioning assessment to be non-invasively and readily obtained for measuring changes in heart chambers. For example, the determination of atrial or ventricular stroke volumes defined by equation E37, ejection fractions defined by equation E38, and cardiac output defined by equation E39. Additionally, the inter-chamber wall volumes (ICWV), thicknesses (ICWT), masses (ICWM) and external cardiac wall volumes, thicknesses, and masses may be similarly determined from the segmentation results obtained by the level set algorithms. Similarly, these accurate, efficient and robust results may be obtained in 2D+time scenarios in situation in which the same scan plane or scan planes is/are sequentially measured in defined periods.

While the particular embodiments have been illustrated and described for determination of ICWT, ICWM, and left and right cardiac ventricular ejection fractions using trained algorithms applied to 3D data sets from 3D transthoracic echocardiograms (TTE), many changes can be made without departing from the spirit and scope of the invention. For example, applications of the disclosed embodiments may be acquired from other regions of interest having a dynamically repeatable cycle. For example, changes in lung movement. Accordingly, the scope of embodiments of the invention is not limited by the disclosure of the particular embodiments. Instead, embodiments of the invention should be determined entirely by reference to the claims that follow.

We claim:

1. A method to determine cardiac ejection volume of a heart comprising:
positioning an ultrasound transceiver to probe a first portion of a heart of a patient, a transceiver adapted to obtain 3D images;
recording a first 3D image during an end-systole time point;
recording a second 3D image during an end-diastole time point;
enhancing the images of a heart in a 3D images with a plurality of algorithms;
measuring the volume of a left ventricle from the enhanced images of a first and second 3D images; and
calculating a change in volume of a left ventricle between a first and second 3D images.

2. A method to determine cardiac ejection volume comprising:
positioning an ultrasound transceiver to probe a first portion of a heart of a patient, to obtain a first 3D images at the end-systole time point;
re-positioning the ultrasound transceiver to probe a second portion of a heart to obtain a second 3D image at the end-diastole time point;
enhancing the images of a heart in a 3D images with a plurality of algorithms;
registering the scanplanes of a first 3D image with a second 3D image;
associating the registered scanplanes into a composite array;
determining the change in volume of a left ventricle of a heart in the composite array.

3. The method of claim 1, wherein plurality of scanplanes are acquired from a rotational array, a translational array, or a wedge array.

4. A system for determining cardiac ejection fraction of a subject comprising:
an electrocardiograph in signal communication with the subject to determine the end-systole and end-diastole time points of the subject;
an ultrasound transceiver in signal communication with the electrocardiograph and positioned to acquire 3D images at the end-systole and the end-diastole time points determined by the electrocardiograph;
a computer system in communication with the transceiver, a computer system having a microprocessor and a memory, the memory further containing stored programming instructions operable by the microprocessor to associate the plurality of scanplanes of each array, and
the memory further containing instructions operable by the microprocessor to determine the change in volume of a left ventricle of a heart at the end systole and end diastole time points.

5. The system of claim 4, wherein change in volume is calculated as a percentage.

6. The system of claim 4, wherein the array includes rotational, wedge, and translation.

7. The system of claim 4, wherein stored programming instructions further include aligning scanplanes having overlapping regions from each location into a plurality of registered composite scanplanes.

8. The system of claim 7, wherein the stored programming instructions further include fusing the registered composite scanplanes cardiac regions of the scanplanes of each array.

9. The system of claim 8, wherein the stored programming instructions further include arranging the fused composite scanplanes into a composite array.

10. The system of claim 4, wherein a computer system is configured for remote operation via a local area network or an Internet web-based system, the internet web-based system having a plurality of programs that collect, analyze, determine and store cardiac ejection fraction measurements.

11. A method for cardiac imaging, comprising:
creating a database of 3D images having manually segmented regions;
training level-set image processing algorithms to substantially reproduce the shapes of the manually segmented regions using a computer readable medium;
acquiring a non-database 3D image;
segmenting the regions of the non-database image by applying the trained level-set processing algorithms using the computer readable medium, and
determining from the segmented non-database 3D image at least one of:
a volume of any heart chamber, and
a thickness of the wall between any adjoining heart chambers.

12. A system for cardiac imaging comprising:
a database of 3D images having manually segmented regions;
an ultrasound transceiver configured to deliver ultrasound pulses into and acquire ultrasound echoes from a subject as 3D image data sets;
an electrocardiograph to determine the timing to acquire the 3D data sets; and
a computer readable medium configured to train level-set image processing algorithms to substantially reproduce the shapes of the manually segmented regions and to segment regions of interest of the 3D data sets using the trained algorithms,
wherein at least one cardiac metric from the 3D data sets is determined from the segmented regions of interest.
